SEQUENCE LISTING (61 sequences)

SEQ ID NO: 1 - DNA, 783 bp, synthetic construct
  misc_feature 1..783: note = RBG1 CDS seq
  source 1..783: mol_type = other DNA
atggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattga

SEQ ID NO: 2 - DNA, 783 bp, synthetic construct
  misc_feature 1..783: note = RBG2 CDS seq
  source 1..783: mol_type = other DNA
atggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattga

SEQ ID NO: 3 - DNA, 477 bp, synthetic construct
  misc_feature 1..477: note = RBG3 CDS seq
  source 1..477: mol_type = other DNA
atggttagcaagggggaggaggttattaaagaattcccaggaagcacccggtggaatccaacggcggagcaactttcgctccttcgggaaatttattacaggaatgggctgagaactcccaccgctgacgagatcagacagatttcatcaaaattgtcgcggtatggtaagattgaagggaaaaatgtgtacaactggttccagaacagaagagctagggaaaagagaaaacagcggttgtcaaccataggctgtggttcggcggggtcggccgcagggagcggagagtttcgccatgtgaagaccctgccccttttccctttgcataataatgaggattcaggttcgttggatctggagctggaattgggttttgcctctgctaccgcagccgcgacttctggttcacaaagggcgtcggcattggatctctgcctgtcccttggaaatgagtcatgcggtttgcatgacaactga

SEQ ID NO: 4 - DNA, 477 bp, synthetic construct
  misc_feature 1..477: note = RBG4 CDS seq
  source 1..477: mol_type = other DNA
atggtctcgaaaggagaagaggtcatcaaagaattttcaggcagcacccgctggaatccgactgcagaacaactttctctcctcaaagagctttattacagaaatgggatcagaaccccttcagcggaccagatacgccagataagcgcccgcttgtcgaggtacggcaaaatcgaggggaagaatgttttttactggttccaaaaccataaagcgagggagcggcagaagaaaagactgagcactgtgggttgtggatcggcgggatcggctgccgggtcaggtgaatttcgccatgttaaaacgcttccactgtttcccttgcataataatgaagacagcggatcacttgacctggagctggagttgggcttcgcttccgcgacggctgctgcaacatccggctcgcagcgggcgtcggcgctggatttgtgcctttcgctgggaaatgagagctgcggtcttcatgacaactga

SEQ ID NO: 5 - DNA, 501 bp, synthetic construct
  misc_feature 1..501: note = RBG5 CDS seq
  source 1..501: mol_type = other DNA
atggtgtctaaaggggaagaggtcataaaagagttctcaggttctactcgctggaatccaactgcagaacaactctctctcctgaaggagctctactatcggaacggaatacgcacgccttcggcggaccaaataaggcagatttcagcccgcctgtctagatacgggaaaatcgagggaaagaacgtgttttattggttccaaaaccataaggcaagagagcggcagaaaaaacggctcagcacagtcggctgtgggtcagccggttcggcggctggctctggtgaattccgccacgtcaaaaccctgcctctgttcccgctccacaataatgtcgagacacttcccttgttcccggaggattccgggtctctcgacttggagctcgaattgggctttgcctctgccactgcggcagcgaccagcggatcacagagagcctccgcactggatctgtgcctgtcacttggaaatgaatcgtgcggattgcacgacaattga

SEQ ID NO: 6 - DNA, 771 bp, synthetic construct
  misc_feature 1..771: note = RBG6 CDS seq
  source 1..771: mol_type = other DNA
atggtgtctaaaggcgaagaggtcattaaggagttcagcggctctactagatggaatccaacggccgagcaacttagcctgctcaaagaactctattatcgcaatgggatacgcacgccctctgcagatcagatacgccaaatctcagcgcgcctgtccaggtatgggaagattgagggtaaaaacgtcttttactggttccaaaatcacaaagcgcgcgagagacagaagaaaagactcagcaccgttggttgtgaccctgctctcatcgagatggggaatgttgcttccctggagtttggcacagagagcgctttggaatcactctcttccggcccctcaagcgaactcagagaggctcccactcggaagttttacgagaaaaaaaccgtcggagaaaacagcacgattatcaaccccgtcgagcagaactgcaccctctcatgcggcacgtcccaggagtttcagtacgccgtcgactcgaggagagtgatgaaggcgatggaggaaaagcaagcaactgacgacgagcccgacggaaacaagtggactgaatccaatcgccatgttaaaacgttgcctttgttcccgctccataacaatgtggagacgttgccactcttcccggaggactcgggatctctggacttggagctggaactgggctttgcttcggctaccgcagcagccacctcgggttcccagagagcctcggctcttgatctctgcctgtcactgggtaacgaatcttgcggactgcacgataattag

SEQ ID NO: 7 - DNA, 840 bp, synthetic construct
  misc_feature 1..840: note = RBG7 CDS seq
  source 1..840: mol_type = other DNA
atggtttcaaagggggaggaggtcataaaagaattttctggcagcactagatggaatccgactgctgagcagttgtctctcctcaaagaactttactataggaacggtatccgcactccgtccgctgatcaaattcgccaaatttcggccagactgtccagatacgggaaaattgagggaaaaaatgttttctactggttccagaatcataaggcgcgcgaaaggcagaaaaagagactttcgaccgtcggatgtgaccctgcacttatcgaaatgggcaatgtcgcgtcgttggagtttggcaccgaatccgctctcgaatctcttagctcgggcccctcatcggaactcagagaggcccctaccagaaaattttatgagaaaaagacagtgggagagaactctacaatcattaaccccgtcgagcagaactgcacattgagctgtggaacatcgcaggagtttcaatacgcggtggattcgcgccgcgttatgaaggcaatggaggagaagcaggcaacagatgatgagccagacggcaataaatggaccgaatctaacagacatgtcaagacactgccacttttccctctccacaacaatgtcgagacactccctctgtttcccgaggattcgggaagcctggatcttgagctggaacttggctttgcatcaggtggatctgccgggtcagttgacacgctgccattgtttcctagagaggctgcgggttctggggaatttgccactgcggctgcgactagcggatcacagcgcgcatcggcgctcgacctctgcctttctttgggtaacgagtcttgcggcctgcatgacaactga

SEQ ID NO: 8 - DNA, 498 bp, synthetic construct
  misc_feature 1..498: note = RBG8 CDS seq
  source 1..498: mol_type = other DNA
atggtttcgaagggggaagaagtcattaaagagttttcagggtctacgcgctggaaccctaccgctgaacaagtcaaggttttgagggaaatatattaccgcgcgggtcttaggacgccatccgcggatcagatccgccaaatatcctcacagctgaaacggtatggaaaggtggaaggaaaaaacgtgttttattggttccagaaccataaagcgcgggaaagacaaaaaaagcggctcagcaccgttgggagcgctggctcagcagcgggttcaggtgagttcaggcacgttaaaacactgcccttgttccctttgcacaataatgaagactcagggagcggtttggacttggatctgaaccttgagttgcgcctgtcggcgactgctgctgccacttcgggctcacagcgggcgtctgccctcgatctggacttggagctcagactcggtttcgcgcttggtaatgagtcgtgtggactgcacgacaactagtga

SEQ ID NO: 9 - DNA, 2040 bp, Zea mays
  source 1..2040: mol_type = unassigned DNA
atggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctatccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtggccggcggcagcgccagcggagcggcctcggagagcacgtcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagagaagggcagagtcgcaagggtaggcaagtttaccttggtggctatgacaaggaggacaaggcagcaagggcttacgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggcgcggccaggcgcctcaaggacgccgtggaccacgtggaggccggcgccactatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcccacagcctgcaggacctccagcagctgcacctcggcagcgcggcccacaacttcttccaggcgtcgtcgagctccacagtctacaacggcggcgccggcgccagtggtgggtaccagggcctcggtggtggtagctccttcctcatgccgtcgagcactgtcgtggcggcggccgaccaggggcacagcagcacggcaaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 10 - DNA, 1485 bp, Zea mays
  source 1..1485: mol_type = unassigned DNA
atggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatga

SEQ ID NO: 11 - DNA, 1470 bp, Zea mays
  source 1..1470: mol_type = unassigned DNA
atggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtag

SEQ ID NO: 12 - protein, 260 aa, synthetic construct
  REGION 1..260: note = protein RBP1 encoded by synthesized SEQ ID NO: 1
MESGSGTAAGSGYVYRQPGSTRWNPTAEQLSLLREIYYRNGLRTPTADEIRQISSKLSRYGKIEGKNVYNWFQNRRAREKRKQRLSTIGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKILQLFPLHNNEDQTLIKSDKEIYCLGSCEKKMDLSPLGHSGSQRASALDLCLSLGNESCGLHDN

SEQ ID NO: 13 - protein, 260 aa, synthetic construct
  REGION 1..260: note = protein RBP2 encoded by synthesized SEQ ID NO: 2
MESGSGTAAGSGYVYRQSGSTRWNPTAEQLSLLKELYYRNGIRTPSADQIRQISARLSRYGKIEGKNVFYWFQNHKARERQKKRLSTVGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKTLPLFPLHNNEDQTLIKSDKEIYCLGSCEKKMDLSPLGHSGSQRASALDLCLSLGNESCGLHDN

SEQ ID NO: 14 - protein, 158 aa, synthetic construct
  REGION 1..158: note = protein RBP3 encoded by synthesized SEQ ID NO: 3
MVSKGEEVIKEFPGSTRWNPTAEQLSLLREIYYRNGLRTPTADEIRQISSKLSRYGKIEGKNVYNWFQNRRAREKRKQRLSTIGCGSAGSAAGSGEFRHVKTLPLFPLHNNEDSGSLDLELELGFASATAAATSGSQRASALDLCLSLGNESCGLHDN

SEQ ID NO: 15 - protein, 158 aa, synthetic construct
  REGION 1..158: note = protein RBP4 encoded by synthesized SEQ ID NO: 4
MVSKGEEVIKEFSGSTRWNPTAEQLSLLKELYYRNGIRTPSADQIRQISARLSRYGKIEGKNVFYWFQNHKARERQKKRLSTVGCGSAGSAAGSGEFRHVKTLPLFPLHNNEDSGSLDLELELGFASATAAATSGSQRASALDLCLSLGNESCGLHDN

SEQ ID NO: 16 - protein, 166 aa, synthetic construct
  REGION 1..166: note = protein RBP5 encoded by synthesized SEQ ID NO: 5
MVSKGEEVIKEFSGSTRWNPTAEQLSLLKELYYRNGIRTPSADQIRQISARLSRYGKIEGKNVFYWFQNHKARERQKKRLSTVGCGSAGSAAGSGEFRHVKTLPLFPLHNNVETLPLFPEDSGSLDLELELGFASATAAATSGSQRASALDLCLSLGNESCGLHDN

SEQ ID NO: 17 - protein, 256 aa, synthetic construct
  REGION 1..256: note = protein RBP6 encoded by synthesized SEQ ID NO: 6
MVSKGEEVIKEFSGSTRWNPTAEQLSLLKELYYRNGIRTPSADQIRQISARLSRYGKIEGKNVFYWFQNHKARERQKKRLSTVGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKTLPLFPLHNNVETLPLFPEDSGSLDLELELGFASATAAATSGSQRASALDLCLSLGNESCGLHDN

SEQ ID NO: 18 - protein, 279 aa, synthetic construct
  REGION 1..279: note = protein RBP7 encoded by synthesized SEQ ID NO: 7
MVSKGEEVIKEFSGSTRWNPTAEQLSLLKELYYRNGIRTPSADQIRQISARLSRYGKIEGKNVFYWFQNHKARERQKKRLSTVGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKTLPLFPLHNNVETLPLFPEDSGSLDLELELGFASGGSAGSVDTLPLFPREAAGSGEFATAAATSGSQRASALDLCLSLGNESCGLHDN

SEQ ID NO: 19 - protein, 164 aa, synthetic construct
  REGION 1..164: note = protein RBP8 encoded by synthesized SEQ ID NO: 8
MVSKGEEVIKEFSGSTRWNPTAEQVKVLREIYYRAGLRTPSADQIRQISSQLKRYGKVEGKNVFYWFQNHKARERQKKRLSTVGSAGSAAGSGEFRHVKTLPLFPLHNNEDSGSGLDLDLNLELRLSATAAATSGSQRASALDLDLELRLGFALGNESCGLHDN

SEQ ID NO: 20 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVAGGSASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 21 - protein, 494 aa, Zea mays
MDTSHHYHPWLNFSLAHHCDLEEEERGAAAELAAIAGAAPPPKLEDFLGGGVATGGPEAVAPAEMYDSDLKFIAAAGFLGGSAAAAATSPLSSLDQAGSKLALPAAAAAPAPEQRKAVDSFGQRTSIYRGVTRHRWTGRYEAHLWDNSCRREGQSRKGRQVYLGGYDKEEKAARAYDLAALKYWGSSTTTNFPVAEYEKEVEEMKNMTRQEFVASLRRKSSGFSRGASIYRGVTRHHQHGRWQARIGRVAGNKDLYLGTFSTEEEAAEAYDIAAIKFRGLNAVTNFEISRYNVETIMSSNLPVASMSSSSAAAAGGRSSKALESPPSGSLDGGGGMPVVEGSTAPPLFIPVKYDQQQQEYLSMLALQHHHQQQQAGNLLQGPLVGFGGLYSSGVNLDFANSHGTAAPSSMAHHCYANGTASASHEHQHQHQMQQGGENETQPQPQQSSSSCSSLPFATPVAFNGSYESSITAAGPFGYSYPNVAAFQTPIYGME

SEQ ID NO: 22 - protein, 489 aa, Zea mays
MDMDMSSAYPHHWLSFSLSNNYHHGLLEAFSNSSGTPLGDEPGAVEESPRTVEDFLGGVGGAGAPPQPAAAADQDHQLVCGELGSITARFLRHYPAAPAGTTVENPGAVTVAAMSSTDVAGAESDQARRPAETFGQRTSIYRGVTRHRWTGRYEAHLWDNSCRREGQSRKGRQVYLGGYDKEEKAARAYDLAALKYWGPTTTTNFPVSNYEKELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVESILSSDLPVGGGATGRAAKFPLDSLQPGSAAAMMLAGAAAASQATMPPSEKDYWSLLALHYQQQQEQERQFPASAYEAYGSGGVNVDFTMGTSSGNNNNNTGSGVMWGATTGAVVVGQQDSSGKQGNGYASNIPYAAAAAMVSGSAGYEGSTGDNGTWVTTTITSSNTGTAPHYYNYLFGME

SEQ ID NO: 23 - DNA, 2961 bp, synthetic construct
  misc_feature 1..2961: note = recombinant BdEF1_RBG1_expression_cassette
  source 1..2961: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 24 - DNA, 2961 bp, synthetic construct
  misc_feature 1..2961: note = recombinant BdEF1_RBG2_expression_cassette
  source 1..2961: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 25 - DNA, 2655 bp, synthetic construct
  misc_feature 1..2655: note = recombinant BdEF1_RBG3_expression_cassette
  source 1..2655: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggttagcaagggggaggaggttattaaagaattcccaggaagcacccggtggaatccaacggcggagcaactttcgctccttcgggaaatttattacaggaatgggctgagaactcccaccgctgacgagatcagacagatttcatcaaaattgtcgcggtatggtaagattgaagggaaaaatgtgtacaactggttccagaacagaagagctagggaaaagagaaaacagcggttgtcaaccataggctgtggttcggcggggtcggccgcagggagcggagagtttcgccatgtgaagaccctgccccttttccctttgcataataatgaggattcaggttcgttggatctggagctggaattgggttttgcctctgctaccgcagccgcgacttctggttcacaaagggcgtcggcattggatctctgcctgtcccttggaaatgagtcatgcggtttgcatgacaactgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 26 - DNA, 2655 bp, synthetic construct
  misc_feature 1..2655: note = recombinant BdEF1_RBG4_expression_cassette
  source 1..2655: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtctcgaaaggagaagaggtcatcaaagaattttcaggcagcacccgctggaatccgactgcagaacaactttctctcctcaaagagctttattacagaaatgggatcagaaccccttcagcggaccagatacgccagataagcgcccgcttgtcgaggtacggcaaaatcgaggggaagaatgttttttactggttccaaaaccataaagcgagggagcggcagaagaaaagactgagcactgtgggttgtggatcggcgggatcggctgccgggtcaggtgaatttcgccatgttaaaacgcttccactgtttcccttgcataataatgaagacagcggatcacttgacctggagctggagttgggcttcgcttccgcgacggctgctgcaacatccggctcgcagcgggcgtcggcgctggatttgtgcctttcgctgggaaatgagagctgcggtcttcatgacaactgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 27 - DNA, 2679 bp, synthetic construct
  misc_feature 1..2679: note = recombinant BdEF1_RBG5_expression_cassette
  source 1..2679: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtgtctaaaggggaagaggtcataaaagagttctcaggttctactcgctggaatccaactgcagaacaactctctctcctgaaggagctctactatcggaacggaatacgcacgccttcggcggaccaaataaggcagatttcagcccgcctgtctagatacgggaaaatcgagggaaagaacgtgttttattggttccaaaaccataaggcaagagagcggcagaaaaaacggctcagcacagtcggctgtgggtcagccggttcggcggctggctctggtgaattccgccacgtcaaaaccctgcctctgttcccgctccacaataatgtcgagacacttcccttgttcccggaggattccgggtctctcgacttggagctcgaattgggctttgcctctgccactgcggcagcgaccagcggatcacagagagcctccgcactggatctgtgcctgtcacttggaaatgaatcgtgcggattgcacgacaattgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 28 - DNA, 2949 bp, synthetic construct
  misc_feature 1..2949: note = recombinant BdEF1_RBG6_expression_cassette
  source 1..2949: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtgtctaaaggcgaagaggtcattaaggagttcagcggctctactagatggaatccaacggccgagcaacttagcctgctcaaagaactctattatcgcaatgggatacgcacgccctctgcagatcagatacgccaaatctcagcgcgcctgtccaggtatgggaagattgagggtaaaaacgtcttttactggttccaaaatcacaaagcgcgcgagagacagaagaaaagactcagcaccgttggttgtgaccctgctctcatcgagatggggaatgttgcttccctggagtttggcacagagagcgctttggaatcactctcttccggcccctcaagcgaactcagagaggctcccactcggaagttttacgagaaaaaaaccgtcggagaaaacagcacgattatcaaccccgtcgagcagaactgcaccctctcatgcggcacgtcccaggagtttcagtacgccgtcgactcgaggagagtgatgaaggcgatggaggaaaagcaagcaactgacgacgagcccgacggaaacaagtggactgaatccaatcgccatgttaaaacgttgcctttgttcccgctccataacaatgtggagacgttgccactcttcccggaggactcgggatctctggacttggagctggaactgggctttgcttcggctaccgcagcagccacctcgggttcccagagagcctcggctcttgatctctgcctgtcactgggtaacgaatcttgcggactgcacgataattagaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 29 - DNA, 3018 bp, synthetic construct
  misc_feature 1..3018: note = recombinant BdEF1_RBG7 expression_cassette
  source 1..3018: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtttcaaagggggaggaggtcataaaagaattttctggcagcactagatggaatccgactgctgagcagttgtctctcctcaaagaactttactataggaacggtatccgcactccgtccgctgatcaaattcgccaaatttcggccagactgtccagatacgggaaaattgagggaaaaaatgttttctactggttccagaatcataaggcgcgcgaaaggcagaaaaagagactttcgaccgtcggatgtgaccctgcacttatcgaaatgggcaatgtcgcgtcgttggagtttggcaccgaatccgctctcgaatctcttagctcgggcccctcatcggaactcagagaggcccctaccagaaaattttatgagaaaaagacagtgggagagaactctacaatcattaaccccgtcgagcagaactgcacattgagctgtggaacatcgcaggagtttcaatacgcggtggattcgcgccgcgttatgaaggcaatggaggagaagcaggcaacagatgatgagccagacggcaataaatggaccgaatctaacagacatgtcaagacactgccacttttccctctccacaacaatgtcgagacactccctctgtttcccgaggattcgggaagcctggatcttgagctggaacttggctttgcatcaggtggatctgccgggtcagttgacacgctgccattgtttcctagagaggctgcgggttctggggaatttgccactgcggctgcgactagcggatcacagcgcgcatcggcgctcgacctctgcctttctttgggtaacgagtcttgcggcctgcatgacaactgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 30 - DNA, 2676 bp, synthetic construct
  misc_feature 1..2676: note = recombinant BdEF1_RBG8_expression_cassette
  source 1..2676: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtttcgaagggggaagaagtcattaaagagttttcagggtctacgcgctggaaccctaccgctgaacaagtcaaggttttgagggaaatatattaccgcgcgggtcttaggacgccatccgcggatcagatccgccaaatatcctcacagctgaaacggtatggaaaggtggaaggaaaaaacgtgttttattggttccagaaccataaagcgcgggaaagacaaaaaaagcggctcagcaccgttgggagcgctggctcagcagcgggttcaggtgagttcaggcacgttaaaacactgcccttgttccctttgcacaataatgaagactcagggagcggtttggacttggatctgaaccttgagttgcgcctgtcggcgactgctgctgccacttcgggctcacagcgggcgtctgccctcgatctggacttggagctcagactcggtttcgcgcttggtaatgagtcgtgtggactgcacgacaactagtgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 31 - DNA, 4224 bp, synthetic construct
  misc_feature 1..4224: note = recombinant BdEF1_ZmPL T3_expression_cassette
  source 1..4224: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatcccccgggatggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctatccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtggccggcggcagcgccagcggagcggcctcggagagcacgtcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagagaagggcagagtcgcaagggtaggcaagtttaccttggtggctatgacaaggaggacaaggcagcaagggcttacgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggcgcggccaggcgcctcaaggacgccgtggaccacgtggaggccggcgccactatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcccacagcctgcaggacctccagcagctgcacctcggcagcgcggcccacaacttcttccaggcgtcgtcgagctccacagtctacaacggcggcgccggcgccagtggtgggtaccagggcctcggtggtggtagctccttcctcatgccgtcgagcactgtcgtggcggcggccgaccaggggcacagcagcacggcaaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 32 - DNA, 3663 bp, synthetic construct
  misc_feature 1..3663: note = recombinant BdEF1_ZmPL T5_expression_cassette
  source 1..3663: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 33 - DNA, 3648 bp, synthetic construct
  misc_feature 1..3648: note = recombinant BdEF1_ZmPL T7 expression_cassette
  source 1..3648: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtagaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 34 - DNA, 5100 bp, synthetic construct
  misc_feature 1..5100: note = recombinant pABM-BdEF1_expression_cassette
  source 1..5100: mol_type = other DNA
gatcccccgggctgcaggaattcaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttg

SEQ ID NO: 35 - DNA, 5865 bp, synthetic construct
  misc_feature 1..5865: note = recombinant pABM-BdEF1_RBG1
  source 1..5865: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattgaa

SEQ ID NO: 36 - DNA, 5865 bp, synthetic construct
  misc_feature 1..5865: note = recombinant pABM-BdEF1_RBG2
  source 1..5865: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattgaa

SEQ ID NO: 37 - DNA, 5559 bp, synthetic construct
  misc_feature 1..5559: note = recombinant pABM-BdEF1_RBG3
  source 1..5559: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggttagcaagggggaggaggttattaaagaattcccaggaagcacccggtggaatccaacggcggagcaactttcgctccttcgggaaatttattacaggaatgggctgagaactcccaccgctgacgagatcagacagatttcatcaaaattgtcgcggtatggtaagattgaagggaaaaatgtgtacaactggttccagaacagaagagctagggaaaagagaaaacagcggttgtcaaccataggctgtggttcggcggggtcggccgcagggagcggagagtttcgccatgtgaagaccctgccccttttccctttgcataataatgaggattcaggttcgttggatctggagctggaattgggttttgcctctgctaccgcagccgcgacttctggttcacaaagggcgtcggcattggatctctgcctgtcccttggaaatgagtcatgcggtttgcatgacaactgaa

SEQ ID NO: 38 - DNA, 5559 bp, synthetic construct
  misc_feature 1..5559: note = recombinant pABM-BdEF1_RBG4
  source 1..5559: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtctcgaaaggagaagaggtcatcaaagaattttcaggcagcacccgctggaatccgactgcagaacaactttctctcctcaaagagctttattacagaaatgggatcagaaccccttcagcggaccagatacgccagataagcgcccgcttgtcgaggtacggcaaaatcgaggggaagaatgttttttactggttccaaaaccataaagcgagggagcggcagaagaaaagactgagcactgtgggttgtggatcggcgggatcggctgccgggtcaggtgaatttcgccatgttaaaacgcttccactgtttcccttgcataataatgaagacagcggatcacttgacctggagctggagttgggcttcgcttccgcgacggctgctgcaacatccggctcgcagcgggcgtcggcgctggatttgtgcctttcgctgggaaatgagagctgcggtcttcatgacaactgaa

SEQ ID NO: 39 - DNA, 5583 bp, synthetic construct
  misc_feature 1..5583: note = recombinant pABM-BdEF1_RBG5
  source 1..5583: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtgtctaaaggggaagaggtcataaaagagttctcaggttctactcgctggaatccaactgcagaacaactctctctcctgaaggagctctactatcggaacggaatacgcacgccttcggcggaccaaataaggcagatttcagcccgcctgtctagatacgggaaaatcgagggaaagaacgtgttttattggttccaaaaccataaggcaagagagcggcagaaaaaacggctcagcacagtcggctgtgggtcagccggttcggcggctggctctggtgaattccgccacgtcaaaaccctgcctctgttcccgctccacaataatgtcgagacacttcccttgttcccggaggattccgggtctctcgacttggagctcgaattgggctttgcctctgccactgcggcagcgaccagcggatcacagagagcctccgcactggatctgtgcctgtcacttggaaatgaatcgtgcggattgcacgacaattgaa

SEQ ID NO: 40 - DNA, 5853 bp, synthetic construct
  misc_feature 1..5853: note = recombinant pABM-BdEF1_RBG6
  source 1..5853: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtgtctaaaggcgaagaggtcattaaggagttcagcggctctactagatggaatccaacggccgagcaacttagcctgctcaaagaactctattatcgcaatgggatacgcacgccctctgcagatcagatacgccaaatctcagcgcgcctgtccaggtatgggaagattgagggtaaaaacgtcttttactggttccaaaatcacaaagcgcgcgagagacagaagaaaagactcagcaccgttggttgtgaccctgctctcatcgagatggggaatgttgcttccctggagtttggcacagagagcgctttggaatcactctcttccggcccctcaagcgaactcagagaggctcccactcggaagttttacgagaaaaaaaccgtcggagaaaacagcacgattatcaaccccgtcgagcagaactgcaccctctcatgcggcacgtcccaggagtttcagtacgccgtcgactcgaggagagtgatgaaggcgatggaggaaaagcaagcaactgacgacgagcccgacggaaacaagtggactgaatccaatcgccatgttaaaacgttgcctttgttcccgctccataacaatgtggagacgttgccactcttcccggaggactcgggatctctggacttggagctggaactgggctttgcttcggctaccgcagcagccacctcgggttcccagagagcctcggctcttgatctctgcctgtcactgggtaacgaatcttgcggactgcacgataattaga

SEQ ID NO: 41 - DNA, 5922 bp, synthetic construct
  misc_feature 1..5922: note = recombinant pABM-BdEF1_RBG7
  source 1..5922: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtttcaaagggggaggaggtcataaaagaattttctggcagcactagatggaatccgactgctgagcagttgtctctcctcaaagaactttactataggaacggtatccgcactccgtccgctgatcaaattcgccaaatttcggccagactgtccagatacgggaaaattgagggaaaaaatgttttctactggttccagaatcataaggcgcgcgaaaggcagaaaaagagactttcgaccgtcggatgtgaccctgcacttatcgaaatgggcaatgtcgcgtcgttggagtttggcaccgaatccgctctcgaatctcttagctcgggcccctcatcggaactcagagaggcccctaccagaaaattttatgagaaaaagacagtgggagagaactctacaatcattaaccccgtcgagcagaactgcacattgagctgtggaacatcgcaggagtttcaatacgcggtggattcgcgccgcgttatgaaggcaatggaggagaagcaggcaacagatgatgagccagacggcaataaatggaccgaatctaacagacatgtcaagacactgccacttttccctctccacaacaatgtcgagacactccctctgtttcccgaggattcgggaagcctggatcttgagctggaacttggctttgcatcaggtggatctgccgggtcagttgacacgctgccattgtttcctagagaggctgcgggttctggggaatttgccactgcggctgcgactagcggatcacagcgcgcatcggcgctcgacctctgcctttctttgggtaacgagtcttgcggcctgcatgacaactgaa

SEQ ID NO: 42 - DNA, 5580 bp, synthetic construct
  misc_feature 1..5580: note = recombinant pABM-BdEF1_RBG8
  source 1..5580: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggtttcgaagggggaagaagtcattaaagagttttcagggtctacgcgctggaaccctaccgctgaacaagtcaaggttttgagggaaatatattaccgcgcgggtcttaggacgccatccgcggatcagatccgccaaatatcctcacagctgaaacggtatggaaaggtggaaggaaaaaacgtgttttattggttccagaaccataaagcgcgggaaagacaaaaaaagcggctcagcaccgttgggagcgctggctcagcagcgggttcaggtgagttcaggcacgttaaaacactgcccttgttccctttgcacaataatgaagactcagggagcggtttggacttggatctgaaccttgagttgcgcctgtcggcgactgctgctgccacttcgggctcacagcgggcgtctgccctcgatctggacttggagctcagactcggtttcgcgcttggtaatgagtcgtgtggactgcacgacaactagtgaa

SEQ ID NO: 43 - DNA, 7128 bp, synthetic construct
  misc_feature 1..7128: note = recombinant pABM-BdEF1_ZmPL T3
  source 1..7128: mol_type = other DNA
gatcccccgggatggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctatccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtggccggcggcagcgccagcggagcggcctcggagagcacgtcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagagaagggcagagtcgcaagggtaggcaagtttaccttggtggctatgacaaggaggacaaggcagcaagggcttacgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggcgcggccaggcgcctcaaggacgccgtggaccacgtggaggccggcgccactatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcccacagcctgcaggacctccagcagctgcacctcggcagcgcggcccacaacttcttccaggcgtcgtcgagctccacagtctacaacggcggcgccggcgccagtggtgggtaccagggcctcggtggtggtagctccttcctcatgccgtcgagcactgtcgtggcggcggccgaccaggggcacagcagcacggcaaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttg

SEQ ID NO: 44 - DNA, 6567 bp, synthetic construct
  misc_feature 1..6567: note = recombinant pABM-BdEF1_ZmPL T5
  source 1..6567: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatgaa

SEQ ID NO: 45 - DNA, 6552 bp, synthetic construct
  misc_feature 1..6552: note = recombinant pABM-BdEF1_ZmPLT7
  source 1..6552: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtaga

SEQ ID NO: 46 - DNA, 11544 bp, synthetic construct
  misc_feature 1..11544: note = Plasmid pGEP837 MAD7
  source 1..11544: mol_type = other DNA
tttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggggtctcggcgcgccggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtttaaacgaagcaaacatggcatctagcatggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaagaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcggacggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggagagaccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctagattatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttggttccattcttttgtttgtctgccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgtacaccataacagaaagtagtgacaagtgttggccaaggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttacccataaaaaaggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggca

SEQ ID NO: 47 - DNA, 3852 bp, synthetic construct
  misc_feature 1..3852: note = Plasmid pGEP842 sgRNA m7GEP1
  source 1..3852: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtcgtcaaaagacctttttaatttctactcttgtagatctcgtcacgattcccctctccggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 48 - DNA, 11516 bp, synthetic construct
  misc_feature 1..11516: note = Plasmid pGEP1054 Map and Plasmid tdTomato
  source 1..11516: mol_type = other DNA
gggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctcaggtacctctagacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacctgcaggcaagcaagcatgaatgcctgggcgcgccggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtttaaacgaagcaaacatggcatctagcatggctccaaagaagaagaggaaggtcaacaacggcacgaacaacttccagaatttcattggcatttccagcctccagaagacgctccgcaacgccctgatccccaccgagaccacgcagcagttcatcgtgaagaacggcatcatcaaggaggacgagctgcgcggcgagaacaggcagatcctcaaggacatcatggacgactactaccgcggcttcatctccgagacgctctccagcatcgacgacatcgactggacgagcctgttcgagaagatggagatccagctcaagaacggcgacaacaaggacaccctgatcaaggagcagacggagtaccgcaaggccatccacaagaagttcgcgaacgacgacaggttcaagaacatgttctccgccaagctgatcagcgacatcctcccggagttcgtgatccacaacaacaactactccgccagcgagaaggaggagaagacccaggtcatcaagctcttctcccgcttcgcgacgagcttcaaggactacttcaagaacagggccaactgcttctccgcggacgacatctccagctccagctgccacaggatcgtgaacgacaacgccgagatcttcttcagcaacgcgctggtgtaccgcaggatcgtcaagtccctcagcaacgacgacatcaacaagatctccggcgacatgaaggactccctgaaggagatgagcctcgaggagatctacagctacgagaagtacggcgagttcatcacccaggagggcatctccttctacaacgacatctgcggcaaggtcaacagcttcatgaacctctactgccagaagaacaaggagaacaagaacctctacaagctgcagaagctccacaagcagatcctgtgcatcgccgacacgtcctacgaggtgccgtacaagttcgagtccgacgaggaggtgtaccagagcgtcaacggcttcctggacaacatctccagcaagcacatcgtcgagcgcctcaggaagatcggcgacaactacaacggctacaacctggacaagatctacatcgtgagcaagttctacgagtccgtcagccagaagacctaccgcgactgggagacgatcaacacggcgctggagatccactacaacaacatcctccccggcaacggcaagtccaaggccgacaaggtgaagaaggcggtcaagaacgacctccagaagagcatcaccgagatcaacgagctggtgtccaactacaagctctgcagcgacgacaacatcaaggccgagacgtacatccacgagatctcccacatcctgaacaacttcgaggcgcaggagctcaagtacaacccggagatccacctggtcgagtccgagctcaaggccagcgagctgaagaacgtgctcgacgtcatcatgaacgcgttccactggtgcagcgtgttcatgaccgaggagctggtcgacaaggacaacaacttctacgccgagctcgaggagatctacgacgagatctaccccgtgatctccctgtacaacctcgtgcgcaactacgtcacccagaagccgtacagcacgaagaagatcaagctcaacttcggcatcccgaccctggcggacggctggtccaagagcaaggagtactccaacaacgcgatcatcctgatgagggacaacctgtactacctcggcatcttcaacgcgaagaacaagccggacaagaagatcatcgagggcaacacgagcgagaacaagggcgactacaagaagatgatctacaacctcctgccgggccccaacaagatgatcccgaaggtgttcctctccagcaagaccggcgtcgagacgtacaagccctccgcctacatcctggagggctacaagcagaacaagcacatcaagtccagcaaggacttcgacatcaccttctgccacgacctcatcgactacttcaagaactgcatcgcgatccaccccgagtggaagaacttcggcttcgacttctccgacaccagcacgtacgaggacatctccggcttctaccgcgaggtggagctgcagggctacaagatcgactggacgtacatcagcgagaaggacatcgacctcctgcaggagaagggccagctctacctgttccagatctacaacaaggacttctccaagaagagcaccggcaacgacaacctccacacgatgtacctgaagaacctcttctccgaggagaacctgaaggacatcgtcctcaagctgaacggcgaggccgagatcttcttcaggaagtccagcatcaagaacccgatcatccacaagaagggcagcatcctcgtgaaccgcacctacgaggcggaggagaaggaccagttcggcaacatccagatcgtcaggaagaacatccccgagaacatctaccaggagctgtacaagtacttcaacgacaagtccgacaaggagctgagcgacgaggccgcgaagctcaagaacgtggtgggccaccacgaggcggccaccaacatcgtgaaggactaccgctacacgtacgacaagtacttcctgcacatgccgatcaccatcaacttcaaggccaacaagacgggcttcatcaacgacaggatcctgcagtacatcgcgaaggagaaggacctccacgtcatcggcatcgaccgcggcgagaggaacctcatctacgtgtccgtcatcgacacctgcggcaacatcgtggagcagaagagcttcaacatcgtcaacggctacgactaccagatcaagctgaagcagcaggagggcgctaggcagatcgccaggaaggagtggaaggagatcggcaagatcaaggagatcaaggagggctacctctccctcgtgatccacgagatcagcaagatggtcatcaagtacaacgccatcatcgcgatggaggacctctcctacggcttcaagaaggggcgcttcaaggtggagaggcaggtctaccagaagttcgagacgatgctgatcaacaagctgaactacctcgtgttcaaggacatcagcatcaccgagaacggcggcctcctgaagggctaccagctgacgtacatccccgacaagctcaagaacgtgggccaccagtgcggctgcatcttctacgtcccggccgcgtacacctccaagatcgaccccaccacgggcttcgtgaacatcttcaagttcaaggacctcacggtcgacgccaagcgcgagttcatcaagaagttcgactccatccgctacgacagcgagaagaacctgttctgcttcaccttcgactacaacaacttcatcacccagaacaccgtgatgtccaagtccagctggagcgtgtacacgtacggcgtccgcatcaagcgcaggttcgtcaacggcaggttctccaacgagagcgacaccatcgacatcacgaaggacatggagaagaccctcgagatgacggacatcaactggagggacggccacgacctgaggcaggacatcatcgactacgagatcgtgcagcacatcttcgagatcttccgcctcaccgtccagatgaggaactccctgagcgagctcgaggaccgcgactacgacaggctgatctccccggtgctcaacgagaacaacatcttctacgactcggctaaggccggcgacgctctgcccaaggacgcggacgccaacggcgcttactgcatcgcgctgaagggcctctacgagatcaagcagatcacggagaactggaaggaggacggcaagttcagccgcgacaagctcaagattagcaacaaggattggttcgatttcattcagaacaagcggtacctgaagcggccagcggcgactaagaaggcgggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatccc

SEQ ID NO: 49 - DNA, 3852 bp, synthetic construct
  misc_feature 1..3852: note = Plasmid pGEP1067 sgRNA m7GEP22
  source 1..3852: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtcgtcaaaagacctttttaatttctactcttgtagatttcagagcactgcagctaagaggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 50 - DNA, 11413 bp, synthetic construct
  misc_feature 1..11413: note = Construct GEMT121 encoding tdT as marker and LbCpf1
  source 1..11413: mol_type = other DNA
tttgtaggtgccaccttccttttctactgtccttttgatcaagtgacagatagctgggcaatggaatccgaggaggtttcccgatattaccctttgttgaaaagtctcaatagccctttggtcttctgaacctgcaggcaagcaagcatgaatgcctgggcgcgccggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgccttagggaagcaaacatggcatctagcatggcgccgaagaagaagcgcaaggtgtccaagctcgagaagttcacgaactgctactccctctccaagaccctccgcttcaaggccatccccgtgggcaagacccaggagaacatcgacaacaagcgcctcctggtcgaggacgagaagagggcggaggactacaagggcgtgaagaagctcctggaccgctactacctctccttcatcaacgacgtcctgcacagcatcaagctcaagaacctgaacaactacatctccctgttccgcaagaagacgaggaccgagaaggagaacaaggagctcgagaacctggagatcaacctccgcaaggagatcgccaaggcgttcaagggcaacgagggctacaagagcctgttcaagaaggacatcatcgagacgatcctcccggagttcctggacgacaaggacgagatcgccctcgtgaactccttcaacggcttcaccacggcgttcaccggcttcttcgacaaccgcgagaacatgttcagcgaggaggccaagtccacgagcatcgcgttccgctgcatcaacgagaacctgaccaggtacatctccaacatggacatcttcgagaaggtcgacgccatcttcgacaagcacgaggtgcaggagatcaaggagaagatcctcaacagcgactacgacgtcgaggacttcttcgagggcgagttcttcaacttcgtcctgacgcaggagggcatcgacgtgtacaacgccatcatcggtggcttcgtgaccgagtccggcgagaagatcaagggcctcaacgagtacatcaacctgtacaaccagaagaccaagcagaagctcccgaagttcaagcccctctacaagcaggtcctgtccgaccgcgagtccctgagcttctacggcgagggctacacgagcgacgaggaggtcctcgaggtgttcaggaacaccctgaacaagaacagcgagatcttctccagcatcaagaagctcgagaagctgttcaagaacttcgacgagtactccagcgccggcatcttcgtcaagaacggcccggcgatctccacgatcagcaaggatatcttcggcgagtggaacgtgatcagggacaagtggaacgccgagtacgacgacatccacctcaagaagaaggcggtggtcaccgagaagtacgaggacgaccgcaggaagtccttcaagaagatcggctccttcagcctcgagcagctgcaggagtacgccgacgcggacctctccgtggtcgagaagctgaaggagatcatcatccagaaggtcgacgagatctacaaggtgtacggctccagcgagaagctgttcgacgccgacttcgtcctcgagaagtccctgaagaagaacgacgccgtggtcgcgatcatgaaggacctcctggactccgtgaagagcttcgagaactacatcaaggcgttcttcggcgagggcaaggagacgaaccgcgacgagtccttctacggcgacttcgtcctcgcctacgacatcctcctgaaggtggaccacatctacgacgcgatcaggaactacgtgacccagaagccgtacagcaaggacaagttcaagctgtacttccagaacccccagttcatgcgcggctgggacaaggacaaggagacggactaccgcgccaccatcctccgctacggcagcaagtactacctggccatcatggacaagaagtacgcgaagtgcctccagaagatcgacaaggacgacgtcaacggcaactacgagaagatcaactacaagctcctgccgggccccaacaagatgctgccgagggtgttcttctccaagaagtggatggcctactacaaccccagcgaggacatccagaagatctacaagaacggcacgttcaagaagggcgacatgttcaacctcaacgactgccacaagctgatcgacttcttcaaggactccatcagccgctacccgaagtggtccaacgcctacgacttcaacttcagcgagacagagaagtacaaggacatcgcgggcttctacagggaggtcgaggagcagggctacaaggtgtccttcgagtccgccagcaagaaggaggtcgacaagctcgtggaggagggcaagctgtacatgttccagatctacaacaaggacttctccgacaagagccacggcacgcccaacctccacaccatgtacttcaagctcctgttcgacgagaacaaccacggccagatccgcctctccggcggcgccgagctgttcatgaggagggcgagcctcaagaaggaggagctggtggtccaccccgctaacagcccaatcgcgaacaagaacccggacaaccccaagaagaccacgaccctctcctacgacgtgtacaaggacaagcgcttcagcgaggaccagtacgagctgcacatcccgatcgccatcaacaagtgccccaagaacatcttcaagatcaacaccgaggtcagggtgctcctgaagcacgacgacaacccctacgtgatcggcatcgaccgcggcgagaggaacctcctgtacatcgtggtcgtggacggcaagggcaacatcgtggagcagtactccctgaacgagatcatcaacaacttcaacggcatccgcatcaagacggactaccacagcctcctggacaagaaggagaaggagcgcttcgaggccaggcagaactggacctccatcgagaacatcaaggagctcaaggcgggctacatcagccaggtcgtgcacaagatctgcgagctggtcgagaagtacgacgccgtgatcgcgctcgaggacctgaactccggcttcaagaacagcagggtcaaggtggagaagcaggtctaccagaagttcgagaagatgctcatcgacaagctgaactacatggtggacaagaagtccaacccgtgcgctacgggcggcgcgctcaagggctaccagatcaccaacaagttcgagagcttcaagtccatgagcacccagaacggcttcatcttctacatcccggcctggctgacgtccaagatcgaccccagcaccggcttcgtcaacctcctgaagacgaagtacacctccatcgcggacagcaagaagttcatctccagcttcgaccgcatcatgtatgtgccggaggaggacctcttcgagttcgccctggactacaagaacttctccaggacggacgcggattacatcaagaagtggaagctctacagctacggcaaccgcatcaggatcttccgcaaccccaagaagaacaacgtcttcgactgggaggaggtgtgcctcacctccgcctacaaggagctgttcaacaagtacggcatcaactaccagcagggcgacatcagggcgctcctgtgcgagcagagcgacaaggccttctactccagcttcatggcgctcatgtccctcatgctgcagatgcgcaacagcatcacgggcaggaccgacgtcgacttcctgatctccccggtgaagaacagcgacggcatcttctacgacagccgcaactacgaggcccaggagaacgcgatcctgccaaagaacgcggacgccaacggcgcctacaacatcgcgaggaaggtgctgtgggccatcggccagttcaagaaggcggaggacgagaagctcgacaaggtcaagatcgccatctccaacaaggagtggctggagtacgcgcagacctcggtgaagcacaagaggcccgctgccaccaagaaggcgggccaggccaagaagaagaagtgacctcagccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctcaggtacctctagacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggca

SEQ ID NO: 51 - DNA, 3841 bp, synthetic construct
  misc_feature 1..3841: note = Construct GEMT099
  source 1..3841: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatctgttggagaggctaacccccactggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 52 - DNA, 1104 bp, Triticum aestivum
  source 1..1104: mol_type = unassigned DNA
atggagatgcaacaacaatacttcgggggggacggcgatgcggactggttccatcaactcgcattgcttcccccacttccaatctcatcgtctctccccccactcccgatgtcagagggctcatgtctccctatggcagcagcagctgcagctgcactcccccttggcgattgctcgagcgccctcatgatacgccctgaggaacagatgtcttgccttccaatgaacccctctccagcggtcgtcgacgatgtctactcttcctacgcaccgaacaatgtcgacgtgttgccgccattcccggcaggacttgacgacgctctgttgatggagtctttttctgacatcgacctcgaggagtttgctgacgcatttggccacaagatcaagacagaacccctcgacgatgccatggtccccgcggaccacgacttcgcggctcaagcccaacaggcctgccctgtggtcatcatgaatcagcaacaactcaacgcacccagagacgtgcgcctgctcattgacccggatgatgatgacagcaccgtggtggccgggggctatgaagctgcagcggtggggtgcgccgagcagaaacaggtcaggccagcaccacgtagggtgagaaagagctcaggcggcgcaagaccagccgcgggaggaaagtccctcgatcacatcggattcgaggaactcaggacctatttctatatgccaatcaccaaggcagcgagggaaatgaacgtggggctgacagtcctgaagaagagatgccgggaactgggggtggcgcgctggccacacagaaagatgaagtctctgagaagcctgatcctcaacattcaggagatggggaagggcgcaacatctcccgcagccgtgcagggggaacttgaagcgcttgagaggtattgcgccattatggaggagaacccggctatagagctcaccgagcaaacgaagaagctcaggcaggcttgtttcaaagagaattataagcggcgtagagccgccgcttctgttaatcttctcgatcactgctataacgatctggcatctcatgagcagcaaatgcctctcccacaaatgggattctttggattttag

SEQ ID NO: 53 - protein, 367 aa, Triticum aestivum
MEMQQQYFGGDGDADWFHQLALLPPLPISSSLPPLPMSEGSCLPMAAAAAAALPLGDCSSALMIRPEEQMSCLPMNPSPAVVDDVYSSYAPNNVDVLPPFPAGLDDALLMESFSDIDLEEFADAFGHKIKTEPLDDAMVPADHDFAAQAQQACPVVIMNQQQLNAPRDVRLLIDPDDDDSTVVAGGYEAAAVGCAEQKQVRPAPRRVRKSSGGARPAAGGKSLDHIGFEELRTYFYMPITKAAREMNVGLTVLKKRCRELGVARWPHRKMKSLRSLILNIQEMGKGATSPAAVQGELEALERYCAIMEENPAIELTEQTKKLRQACFKENYKRRRAAASVNLLDHCYNDLASHEQQMPLPQMGFFGF

SEQ ID NO: 54 - protein, 25 aa, synthetic construct
  REGION 1..25: note = Description of Artificial Sequence: Synthetic peptide
  VARIANT 2..10: note = Any amino acid
  VARIANT 12..21: note = Any amino acid
  VARIANT 23..24: note = Any amino acid
CXXXXXXXXXCXXXXXXXXXXCXXH

SEQ ID NO: 55 - DNA, 66 bp, synthetic construct
  misc_feature 1..66: note = Description of Artificial Sequence: Synthetic oligonucleotide
  source 1..66: mol_type = other DNA
tttgatttactcgtcacgattcccctctcctggtcgaacttttcaggtggggaaagctgctggcga

SEQ ID NO: 56 - DNA, 44 bp, synthetic construct
  misc_feature 1..44: note = Description of Artificial Sequence: Synthetic oligonucleotide
  source 1..44: mol_type = other DNA
tttgatttactcgtcaggaaagctgctggcgacaggtggaaatc

SEQ ID NO: 57 - DNA, 58 bp, synthetic construct
  misc_feature 1..58: note = Description of Artificial Sequence: Synthetic oligonucleotide
  source 1..58: mol_type = other DNA
tttgatttactcgtcacgattcctggtcgaacttttcaggtggggaaagctgctggcg

SEQ ID NO: 58 - DNA, 44 bp, synthetic construct
  misc_feature 1..44: note = Description of Artificial Sequence: Synthetic oligonucleotide
  source 1..44: mol_type = other DNA
tttgatttacaggtggggaaagctgctggcgacaggtggaaatc

SEQ ID NO: 59 - DNA, 63 bp, synthetic construct
  misc_feature 1..63: note = Description of Artificial Sequence: Synthetic oligonucleotide
  source 1..63: mol_type = other DNA
tttgatttactcgtcacgattcccctctcctggtcgaacttttcaggtggggaaagctgctgg

SEQ ID NO: 60 - DNA, 56 bp, synthetic construct
  misc_feature 1..56: note = Description of Artificial Sequence: Synthetic oligonucleotide
  source 1..56: mol_type = other DNA
tttgatttactcgtcacgattccccgaacttttcaggtggggaaagctgctggcga

SEQ ID NO: 61 - DNA, 56 bp, synthetic construct
  misc_feature 1..56: note = Description of Artificial Sequence: Synthetic oligonucleotide
  source 1..56: mol_type = other DNA
tttgatttactcgtcacgattcccctaacttttcaggtggggaaagctgctggcga

(19) United States
(12) Patent Application Publication
(10) Pub. No.: US 2026/0098273 A1
(43) Pub. Date: Apr. 9, 2026

(54) IMMATURE INFLORESCENCE MERISTEM EDITING

(71) Applicant: KWS SAAT SE & Co. KGaA, Einbeck (DE)

(72) Inventor: Ling MENG, Einbeck (DE)

(73) Assignee: KWS SAAT SE & Co. KGaA, Einbeck (DE)

(21) Appl. No.: 19/358,081

(22) Filed: Oct. 14, 2025

Related U.S. Application Data
(62) Division of application No. 17/799,348, now Pat. No. 12,473,563.
(60) Provisional application No. 62/982,900, filed on Feb. 28, 2020.

(51) Int. Cl. C12N 15/82; C12N 5/00

(52) U.S. Cl. CPC C12N 15/8213; C12N 5/0025; C12N 15/821; C12N 2310/20

(57) ABSTRACT

The present invention relates to a method for plant genome modification of at least one plant cell being in the developmental stage of a plant immature inflorescence meristem (IIM) cell, wherein the modification of the specific cell type is achieved by providing a genome modification or editing system, optionally together with at least one regeneration booster, preferably wherein the effector molecules are introduced by means of particle bombardment. To this end, new artificial and precisely controllable booster genes and proteins are provided. Further, the modified plant cells are regenerated in a direct or an indirect way. Finally, methods, tools, constructs and strategies are provided to effectively modify at least one genomic target site in a plant cell, to obtain said modified cell and to regenerate a, plant tissue, organ, plant or seed from such modified cell.

### CROSS-REFERENCE TO RELATED APPLICATIONS

This application is a divisional of U.S, application Ser. No. 17/799,348, filed Aug. 12, 2022, which is a U.S. National Phase of International Patent Application No. PCT/EP2021/054799, filed on Feb. 26, 2021, which claims priority to U.S. Provisional Application No. 62/982,900, filed Feb. 28, 2020. The entire contents of these applications are incorporated herein by reference in their entirety.

### SEQUENCE LISTING

The instant application contains a Sequence Listing which has been submitted electronically in XML format and is hereby incorporated by reference in its entirety. Said XML copy, created on Sep. 18, 2025, is named 245761_000264_SL.xml and is 242,406 bytes in size.

### TECHNICAL FIELD

The present invention relates to the field of genome engineering or gene editing of specific plant cells. In particular, the present invention relates to the modification of at least one plant cell being in the developmental stage of a plant immature inflorescence meristem (IIM) cell, wherein the modification of the specific cell type is achieved by providing a genome modification or editing system, optionally together with at least one regeneration booster, preferably wherein the effector molecules are introduced by means of particle bombardment. To this end, new artificial and precisely controllable booster genes (RBGs) and proteins (RBPs) are provided. Further, the modified plant cells are regenerated in a direct or an indirect way. Finally, methods, tools, constructs and strategies are provided to effectively modify at least one genomic target site in a plant cell, to obtain said modified cell and to regenerate a, plant tissue, organ, plant or seed from the such modified cell.

### BACKGROUND OF INVENTION

To cope with the increasing challenges of climate change, food safety and a growing world population, traditional plant breeding, usually being rather time consuming, has to be supported by new techniques of molecular biology to provide new crop plants having desired traits in safe manner, but needing less development time.

Having more and more potentially suitable site-specific nuclease tools at hand, transformation or transfection and subsequent regeneration are still the major bottleneck technologies for plant genome engineering, such as genome editing (GE). To obtain a modified plant, the two events have to fall on the same cell. Up to date, particle bombardment and Agrobacterium-mediated biomolecule delivery are the most efficient methods for plant transformation. In agrobacterial transformation, the Agrobacteria first find the suitable cells and attach to the plant cell walls, which is generally referred as “inoculation”. Following the inoculation, the Agrobacteria are growing with plant cells under suitable conditions for a period of time—from several hours to several days—to allow T-DNA transfer. Agrobacterium-plant interaction, plant tissue structure, plant cell type, etc, constrain agrobacterial transformation. Limited by plant cell susceptibility and accessibility it is generally believed that Agrobacterium-mediated transformation is plant species, plant tissue-type and plant cell-type dependent. Conversely, based on physical forces particle bombardment is—at least in theory—plant species and plant cell-type independent, and is able to transform any cells when appropriate pressure applied. Still, many plant cells, in particular plant cells freshly isolated from a plant depending on the developmental stage and the tissue they are derived from, suffer severe stress or even cell death when physically bombarded with micro- or nanoparticles of various kinds. Further, bombardment may be associated with a low transformation and/or integration frequencies also caused by the severe cell damage or rupture. Physical bombardment per se offers great advantages as it is easy, rapid and versatile and allows for transient and stable expression of the inserted molecules, if desired. Potentially toxic chemicals needed for transfection, or bacterial transformations can be avoided.

For genome modification, there is thus a great need in identifying new plant cells and protocols in a suitable developmental stage, which have the capacity to be isolated directly from a living plant, which can be effectively bombarded, e.g., for gene editing, or for any kind of expressing a tool to be inserted stably or transiently, and which can later on be regenerated to a whole plant.

Plant cells are developmentally plastic and likely regenerative. The regenerative capacity of plant cell depends on cell identity, age, and environmental signals. There are at least two types of plant cells: somatic cells and stem cells. Somatic cells are the descendants of a stem cell. They are differentiated cells with specific features morphologically, metabolically, and functionally. The regeneration of somatic cells requires cell fate reprogramming via dedifferentiation into a regenerative cell. On the other hand, plant stem cells are undifferentiated and able to generate new cells, tissues and finally develop into a new plant. Plant stem cells are mainly located on a specialized tissue named plant meristem, including shoot, root meristem, and inflorescence meristem.

For important cereal crops (e.g., maize, wheat, rye, oat, barley, sorghum, rice), the most widely used explant for genome engineering is immature zygotic embryo. The epidermal and sub-epidermal cells from the scutellum surface of immature embryo are ideal recipient cells for Agrobacterium-mediated transformation, and also for particle bombardment. However, the regeneration from the epidermal and sub-epidermal cells on the scutellum surface of immature embryo are highly genotype dependent, and genetic engineering in cereal crops generally rely on several regenerative genotypes, e.g., maize Hi II and A188. Moreover, production of immature zygotic embryos is a time and resource demanding process. It takes at least 12 weeks from seed planting to immature embryo harvesting in maize, and requires well-equipped and highly remained greenhouse conditions and facilities. The quality of immature embryos are also greenhouse and season dependent. Therefore, developing alternative explants that are regenerative and do not rely on long greenhouse periods is highly desirable for genome engineering in cereal crops.

Plants produce abundant inflorescence meristems. An inflorescence meristem is the modified shoot meristem that contains multipotent stem cells and is able to produce floral primordia, and eventually develops into an inflorescence, i.e., a cluster of flowers arranged on a main stem. Today, reliable protocols for efficient plant genome editing are not available for specifically and efficiently transfecting inflorescence meristem, in particular by physical means, to rapidly introduce traits of interest into the genome of a given plant in an inheritable manner.

Another problem in the targeted modification of plants is that it is believed that transformed cells are less regenerable than wild type cells. These circumstances may result in poor rates of genome editing in view of the fact that the transformed/transfected material may not be viable enough after the introduction of the GE tools. For example, transformed cells are susceptible to programmed cell death due to presence of foreign DNA inside of the cells. Stresses arising from delivery (e.g, bombardment damage) may trigger a cell death as well.

Plant development is characterized by repeated initiation of meristems, regions of dividing cells that give rise to new organs. During lateral root (LR) formation, new LR meristems are specified to support the outgrowth of LRs along a new axis. The determination of the sequential events required to form this new growth axis has been hampered by redundant activities of key transcription factors. The effects of three PLETHORA (PLT) transcription factors. PLT3, PLT5, and PLT7, during LR outgrowth were already characterized. It was found that in plt3/plt5/plt7 triple mutants, the morphology of lateral root primordia (LRP), the auxin response gradient, and the expression of meristem/tissue identity markers are impaired from the “symmetry-breaking” periclinal cell divisions during the transition between stage I and stage II, wherein cells first acquire different identities in the proximodistal and radial axes. Particularly, PLT1, PLT2, and PLT4 genes that are typically expressed later than PLT3, PLT5, and PLT7 during LR outgrowth are not induced in the mutant primordia, rendering “PLT-null” LRP. Reintroduction of any PLT clade member in the mutant primordia completely restores layer identities at stage II and rescues mutant defects in meristem and tissue establishment. Therefore, all PLT genes can activate the formative cell divisions that lead to de novo meristem establishment and tissue patterning associated with a new growth axis (Du and Scheres, PNAS 2017, doi.org/10.1073/pnas. 1714410114). Still, the role of PLT proteins and variants thereof in gene editing in specific meristematic cells to promote gene editing in a concerted manner was not described yet.

Again, reliable and efficient protocols are lacking combining the knowledge on plant regeneration boosters with the further powerful gene editing mechanisms, in particular in view of the fact that both techniques require the introduction of huge molecular complexes into a given cell, which has to be in a state susceptible for transformation.

As disclosed in Lowe et al. (Plant Cell, 2016, 28 (9)) there is another problem associated with the use of naturally occurring regeneration boosters in artificial settings of plant genome modifications: the usually growth-stimulating effect of regeneration boosters-if not as precisely controlled as in the natural environment, where the transcription factors are only expressed in a tightly controlled spatio-temporal manner, the ectopic expression of regeneration boosters used in plant genome modification easily leads to pleiotropic effects on plant growth and fertility. These uncertainties and negative effects are, however, not desired for targeted genome editing. To address this problem, Lowe et al, suggests a rather cumbersome technique of integrating and later on inactivation booster activity by removal of the relevant expression cassettes.

Given the current obstacles in highly efficient plant transformation strategies and/or effective site-specific plant genome editing in relevant monocot and dicot plants, it was thus an object of the present invention to provide new plant cell amenable to be transfected/transformed and efficient protocols for transforming specific plant tissue in a defined manner to increase transfection/transformation efficiencies by targeting plant cells in an optimum developmental stage. Finally, it was an object to achieve genome modification, e.g. gene editing, with single-cell origin allowing a homogenous and regenerable genome editing without a conventional selection to speed-up and facilitate current protocols relying on cumbersome and expensive screening and regeneration steps, or suffering from poor and rather singular gene editing events.

### SUMMARY OF THE INVENTION

The above object was achieved by elucidating that plant immature inflorescence meristem (IIM) cells provides an ideal alternative explant for genome engineering and modifications in general and especially for targeted genome editing. The present invention involves direct delivery of biological molecules, e.g. DNA, RNA, protein, RNP, or chemicals into the inflorescence meristem cells as specific target cells, preferably mediated by micro-particle carriers. Following the biolistic delivery of biomolecules, the transformed cells from the immature inflorescence meristem are regenerated in a flexible manner into entire plants via either direct meristem regeneration, or via indirect callus regeneration.

In one aspect, there is provided a method for plant genome modification, preferably for the targeted modification of at least one genomic target sequence, by obtaining a modification of at least one plant immature inflorescence meristem cell, wherein the method comprises the following steps: (a) providing at least one immature inflorescence meristem (IIM) cell: (b) introducing into the at least one immature inflorescence meristem cell: (i) at least one genome modification system, preferably a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same: (ii) optionally: at least one regeneration booster, or a sequence encoding the same, or a regeneration booster chemical, wherein steps (i) and (ii) take place simultaneously, or subsequently, for promoting plant cell proliferation and/or to assist in a targeted modification of at least one genomic target sequence: (iii) and, optionally at least one repair template, or a sequence encoding the same; and (c) cultivating the at least one immature inflorescence meristem cell under conditions allowing the expression and/or assembly of the at least one genome modification system, preferably the at least one genome editing system and optionally the at least one regeneration booster, and optionally of the at least one guide molecule and/or optionally of the at least one repair template; and (d) obtaining at least one modified immature inflorescence meristem cell: or (e) obtaining at least one plant tissue, organ, plant or seed regenerated from the at least one modified cell; and (f) optionally: screening for at least one plant tissue, organ, plant or seed regenerated from the at least one modified cell in the T0 and/or T1 generation carrying a desired targeted modification.

In a further aspect, there are provided isolated nucleic acid sequences, and the polypeptide sequences encoding the same, and recombinant genes, expression cassettes and expression constructs comprising isolated nucleic acid sequences, wherein the polypeptide sequences have the function of a regeneration booster artificially optimized to be perfectly suitable to promote genome modification or gene editing and suitable to be used in combination with at least one further regeneration booster.

In a further aspect, there are provided methods for regenerating recalcitrant plants/plant genotypes using the methods for plant genome modification as provided in the first aspect.

In vet a further aspect, there are provided methods providing at least one regeneration booster, or a specific combination of regeneration boosters, or the sequence(s) encoding the same, for efficiently producing haploid or doubled haploid plant cells, tissues, organs, plants, or seeds.

In one aspect, an IIM cell is preferably transformed by physical bombardment, optionally together with at least one regeneration booster.

In one aspect, the method comprises a regeneration step, wherein the regeneration is direct meristem organogenesis, in another aspect, the regeneration step comprises a step of indirect callus embryogenesis or organogenesis.

In one aspect, the methods specifically rely on the use of at least one regeneration booster, or a sequence encoding the same, or of at least one regeneration booster chemical, wherein the booster fulfils the dual function of enhancing plant regeneration after transfection/transformation and/or of increasing genome modification efficiencies, in particular gene editing efficiencies after inducing a targeted DNA break (single- or double-stranded) by at least one site-directed nuclease.

In a further aspect, specific combinations of regeneration boosters are provided having synergistic activities in promoting plant regeneration and/or genome modification efficiencies, preferably gene editing efficiencies.

In one aspect, particle bombardment is used for transforming or transfecting at least one plant immature inflorescence cell of interest.

In a further aspect, there is provided a plant cell, tissue, organ, plant or seed obtainable by or obtained by a method according to any of the preceding claims.

In yet a further aspect, there is provided the use of a genome modification system, or of a genome editing system for efficiently transforming or transfecting at least one immature inflorescence cell.

In another aspect, expression constructs and expression cassettes are provided encoding the genome modification system, or encoding the genome editing system to be introduced into at least one plant immature inflorescence meristem cell.

Further provided is an expression construct assembly comprising the relevant constructs and cassettes for conducting the methods as disclosed herein.

In a further aspect, methods for staging plants are provided for various relevant crop plants to identify the correct developmental stage when plant immature inflorescence meristem cells are present and thus available for the methods for plant genome modification provided.

In yet another aspect, there is provided a plant cell, preferably an IIM cell, comprising an expression construct assembly, or comprising the recombinant gene, or comprising an expression cassette or an expression construct as disclosed herein, or there is provided a plant tissue, organ, whole plant, or a part thereof or a seed comprising this plant cell.

Further uses of and methods for constructing multiple purpose expression constructs and expression cassettes for use according to the present invention are provided.

### BRIEF DESCRIPTION OF DRAWINGS

Whenever the Figures show black/white pictures of originally fluorescence images, brighter spots represent the accumulation of the respective fluorescent protein.

FIG. 1A-1B (FIG. 1A-1B) shows a deep 50-well plus tray (FIG. 1A) and a 1020 Greenhouse (no holes) tray (FIG. 1B) used for maize seedling cultivation.

FIG. 2 (FIG. 2) shows 28-day-old maize seedlings at late V6 stage growing at 50-well tray in greenhouse are ready for immature tassel harvesting.

FIG. 3A-3C (FIG. 3A-3C) shows freshly isolated immature inflorescences from maize. (FIG. 3A) An immature tassel from a 28-day-old maize A188 seedling: (FIG. 3B) an immature ear from a 36-day-old maize 4V-40171 seedling: (FIG. 3C) an immature inflorescence at AM (anther primordia) stage isolated from a (KWS Bono mature rye plant. GP indicates a glume primordium, and LP for a lemma primordium. An asterisk points to a stamen primordium.

FIG. 4 (FIG. 4) shows a genome editing nuclease MAD7 expression construct pGEP837 map. A green fluorescent marker was used in this example (indicated as GEP). Any kind of fluorescent protein-encoding marker gene may be used instead depending on the plant target cell/tissue to be transformed and visualized. MAD7 defines the maize codon-optimized CDS of the Eubacterium rectale CRISPR/MAD7 gene (Inscripta). BdUBI10 defines the Brachypodium Ubiquitin 10 promoter. Tnos defines the nos terminator.

FIG. 5A-5H (FIG. 5A-5H) shows fluorescence images (a green fluorescent marker gene was used and its expression in the target tissue was visualized accordingly) of maize immature inflorescence 20 hours after bombardment with plasmid pGEP837 (see FIG. 4). (FIG. 5A)-(FIG. 5H): 29-day-old immature tassels from maize inbred lines. (FIG. 5A): Maize elite 4V-40171: (FIG. 5B): maize elite 5V-50269; (FIG. 5C): maize elite 5V-50266: (FIG. 5D): maize elite 3V-30261: (FIG. 5E): maize elite 16V-0089: (FIG. 5F): maize elite 4V-40131: (FIG. 5G): maize elite 2V-20121: (FIG. 5H): maize elite 3V-30315.

FIG. 6 (FIG. 6) shows a genome editing crRNA construct pGEP842 map, m7GEP1 defines the crRNA, which target to maize HMG13 gene. ZmUbi1 defines the promoter and intron from maize Ubiquitin 1 gene. Tnos defines the nos terminator.

FIG. 7 (FIG. 7) shows a maize PLT5 expression construct pABM-BdEF1_ZmPLT5 map. ZMPLT5 is driven by the strong constitutive EFI promoter from Brachypodium (pBdEF1).

FIG. 8A-8D (FIG. 8A-8D) shows a work-flow for genome editing via biolistic bombardment and direct meristem regeneration from immature tassels of maize A188. (FIG. 8A): A fresh isolate immature tassel ready for bombardment: (FIG. 8B): fluorescence images (a green fluorescent marker gene was used and its expression in the target tissue was visualized accordingly) of the immature tassels 20 hours after bombardment with plasmid pGEP837 (see FIG. 4): (FIG. 8C): meristem proliferation step I for 7 days: (FIG. 8D): meristem proliferation step II for 7 days: (FIG. 8E): plantlet development in shooting medium for 7 days: (FIG. 8F): plantlet development in rooting medium for 7 days.

FIG. 9A-9B (FIG. 9A-9B) shows a Sanger sequencing trace decomposition analysis of genome editing events in the regenerated TO plantlets from a 28-day-old A188 immature tassel by direct meristem regeneration. (FIG. 9A) The sequencing results from one of the 12 regenerated plantlets with ˜100% SDN-1 editing (biallelic). FIG. 9A discloses SEQ ID NOS 55-58, respectively, in order of appearance: (FIG. 9B) the sequencing result from the plantlet with ˜50% SDN-1 editing (monoallelic). FIG. 9B discloses SEQ ID NOS 59-61, respectively, in order of appearance.

FIG. 10A-10B (FIG. 10A-10B) shows genome editing SDN-1 by transient biolistic transformation and direct meristem regeneration of immature ears from maize elite 4V-40171 plants harvested at 39 days after planting. (FIG. 10A): A freshly isolated immature car from elite 4V-40171: (FIG. 10B): the immature cars on osmotic medium (IM_OSM) and ready for biolistic bombardment.

FIG. 11 (FIG. 11) shows the KWS_RBP4 expression construct pABM-BdEF1_RBP4 map. KWS_RBP4 is driven by the strong constitutive EFI promoter from Brachypodium (pBdEF1).

FIG. 12 (FIG. 12) shows the KWS_RBP5 expression construct pABM-BdEF1_RBP5 map. KWS_RBP5 is driven by the strong constitutive EFI promoter from Brachypodium (pBdEF1).

FIG. 13A-13F (FIG. 13A-13F) shows the work-flow for genome editing by biolistic transformation and indirect callus regeneration with regeneration boosters from immature tassels of maize A188. (FIG. 13A): A fresh isolate immature tassel ready for bombardment: (FIG. 13B): a fluorescence image (a green fluorescent marker gene was used and its expression in the target tissue was visualized accordingly) of the immature tassels 20 hours after co-bombardment of plasmid pGEP837/pGEP842 with regeneration boosters ZmPLT5 and KWS_RBP4 or KWS_RBP5: (FIG. 13C): callus induced after 20 days in the callus induction medium: (FIG. 13D): callus greening in shooting medium for 5 days: (FIG. 13E): plantlet development in shooting medium for 12 days: (FIG. 13F): plantlets development in rooting medium for 7 days.

FIG. 14 (FIG. 14) shows the KWS_RBP8 expression construct pABM-BdEF1_RBP8 map. KWS_RBP8 is driven by the strong constitutive EFI promoter from Brachypodium (pBdEF1).

FIG. 15 (FIG. 15) shows the pGEP22 expression construct pGEP1067 map, m7GEP22 defines the crRNA, which target to the maize endogenous gene HMG13. ZmUbi1 defines the promoter and intron from maize Ubiquitin 1 gene. Tnos defines the nos terminator.

FIG. 16 (FIG. 16) shows the genome editing nuclease MAD7 expression construct pGEP1054 map, tdTomato defines tdTomato report gene. MAD7 defines the maize codon-optimized CDS of MAD7 nuclease (Inscripta). BdUBI10 defines the Brachypodium Ubiquitin 10 promoter. Tnos defines the nos terminator.

FIG. 17A-17C (FIG. 17A-17C) shows representative images showing stable transformation of the fluorescent report gene tDTomato in corn elites and F1 hybrids with boosters KWS_RBP8, (FIG. 17A): tDTomato expressing calluses indicating the stable transformation event(s) in corn elite MMS18-01495: (FIG. 17B): tDTomato expressing shoot buds indicating the stable transformation event(s) in corn elite PJO-73631: (FIG. 17C): a tDTomato expressing plantlet indicating the stable transformation event(s) in corn F1 hybrid of elite 4V-40171 (♀) x A188 (♂). The fluorescent images shown at the top panel, while the corresponding bright-field images are shown at the bottom panel.

FIG. 18 (FIG. 18) shows a genome editing nuclease Cpf1 expression construct GEMT121 map, tdTomato defines the fluorescent report gene tDTomato driven by double 35S promoter and intron. LbCpf1 defines the maize codon-optimized CDS of Lachnospiraceae bacterium CRISPR/Cpf1 (LbCpf1) gene. BdUBI10 defines the Brachypodium Ubiquitin 10 promoter. Tnos defines the nos terminator.

FIG. 19 (FIG. 19) shows a genome editing crRNA expression construct GEMT099 map, crGEP289 defines the crRNA, which target to wheat CPL3 (C-terminal domain phosphatase-like 3) gene. ZmUbi1 defines the promoter and intron from maize Ubiquitin 1 gene. Tnos defines the nos terminator.

FIG. 20A-20D (FIG. 20A-20D) shows tDTomato fluorescent images of the immature inflorescences from wheat (Triticum aestivum L.) cultivar (Taifun) after co-bombardment with plasmid GEMT121 (FIG. 18) and GEMT099 (FIG. 19). Bright field image (FIG. 20A) or tDTomato fluorescent image (FIG. 20B) of wheat immature inflorescence 16 hours after bombardment: (FIG. 20C): tDTomato fluorescent images of wheat immature inflorescences cultured in embryogenic callus induction medium for 3 days: (FIG. 20D): The number of tDTomato positive structures per immature inflorescence initially used.

FIG. 21A-21C (FIG. 21A-21C) shows images of an immature inflorescence from a 34 day-old sunflower (Helianthus annuus) cultivar velvet Queen plant. (FIG. 21A): the immature inflorescence head with many-pointed star-like appearance at development R1 stage: (FIG. 21B) the fresh isolated immature inflorescence meristem head ready for bombardment: (FIG. 21C): tDTomato fluorescent image of the immature inflorescence head 14 hours after bombarded with plasmid GEMT121 (FIG. 18).

FIG. 22 (FIG. 22) shows KWS_RBP2 expression construct pABM-BdEF1_RBP2 map. KWS_RBP2 is driven by the strong constitutive EFI promoter from Brachypodium (pBdEF1).

FIG. 23A-23D (FIG. 23A-23D) shows biolistic transformation and plant regeneration from cross-section discs of immature center spike of maize A188. A bright field image (FIG. 23A) and a tDTomato fluorescence image (FIG. 23B) of the bombarded discs 16 hours after bombardment. (FIG. 23C) embryogenic calli were induced from the bombarded inflorescence discs 9 days in the callus induction medium. (FIG. 23D) TO plants 7 days in a maize germination phytotray.

FIG. 24 (FIG. 24) shows a genome editing crRNA construct TGCD087 map. Target1_1a defines the crRNA, which target to a maize gene annotated as UV-B-insensitive 4-like gene at target 1. ZmUbi1 defines the promoter and intron from maize Ubiquitin 1 gene. Tnos defines the nos terminator.

FIG. 25 (FIG. 25) shows a genome editing crRNA construct TGCD088 map. Target2_2a defines the crRNA, which target to a maize gene annotated as UV-B-insensitive 4-like gene at target 2. ZmUbi1 defines the promoter and intron from maize Ubiquitin 1 gene. Tnos defines the nos terminator.

FIG. 26 (FIG. 26) shows a genome editing crRNA construct TGCD089 map. Target5_2b defines the crRNA, which target to a maize gene annotated as UV-B-insensitive 4-like gene at target 5. ZmUbi1 defines the promoter and intron from maize Ubiquitin 1 gene. Tnos defines the nos terminator.

FIG. 27 (FIG. 27) shows a genome editing crRNA construct TGCD090 map. Target4_2c defines the crRNA, which target to a maize gene annotated as UV-B-insensitive 4-like gene at target 4. ZmUbi1 defines the promoter and intron from maize Ubiquitin 1 gene. Tnos defines the nos terminator.

FIG. 28 (FIG. 28) shows a genome editing crRNA construct TGCD091 map. Target3_2d defines the crRNA, which target to a maize gene annotated as UV-B-insensitive 4-like gene at target 3. ZmUbi1 defines the promoter and intron from maize Ubiquitin 1 gene. Tnos defines the nos terminator.

FIG. 29A-29B (FIG. 29A-29B) shows multiplex genome editing SDN-1 at 5 target locations of the maize target gene annotated as UV-B-insensitive 4-like gene in A188. (FIG. 29A): maize gene structure and target sequence locations: (FIG. 29B) summary of the multiplex genome editing SDN-1 efficiencies in above maize target gene.

### DESCRIPTION OF SEQUENCES

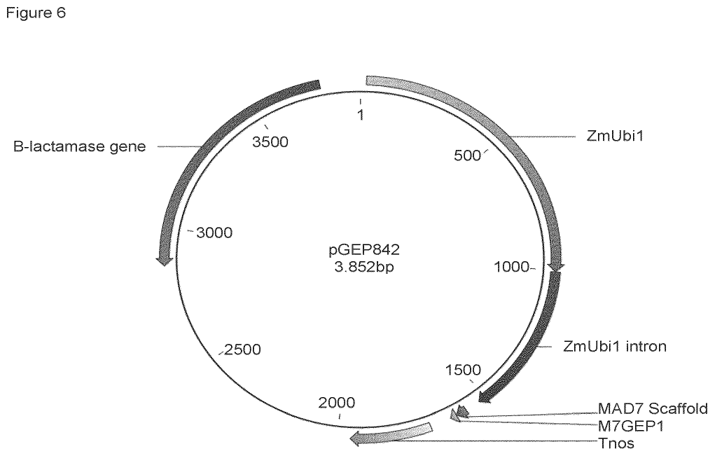

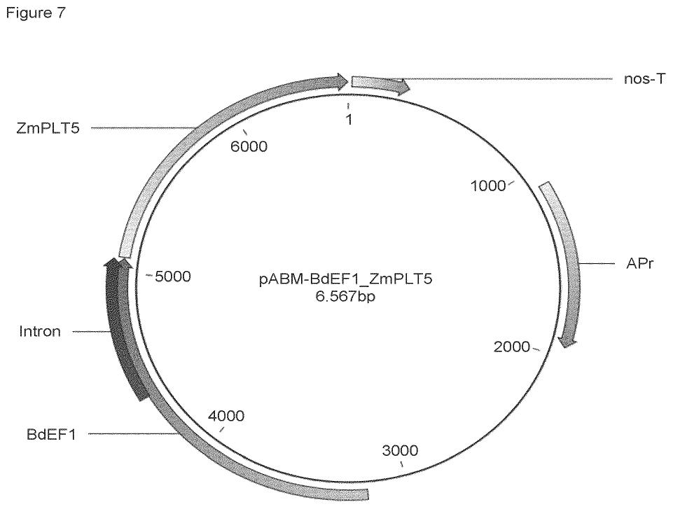

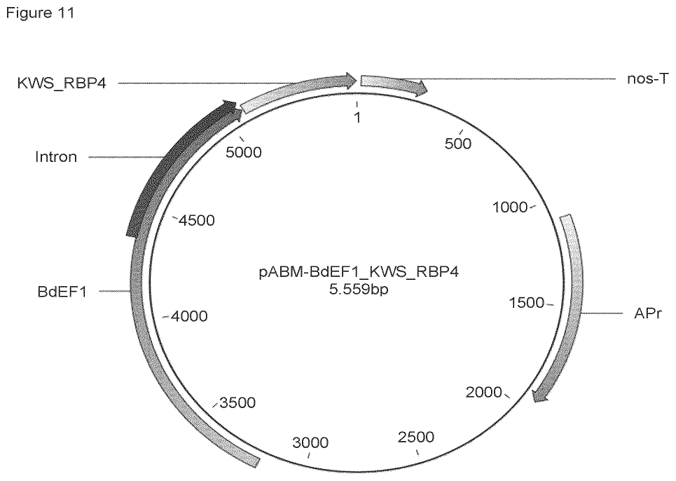

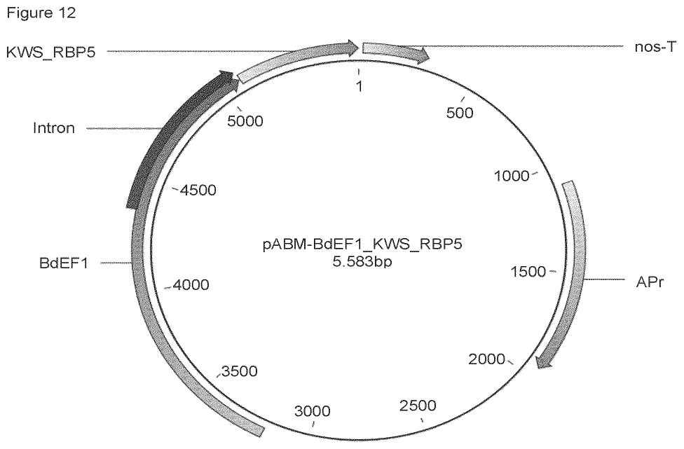

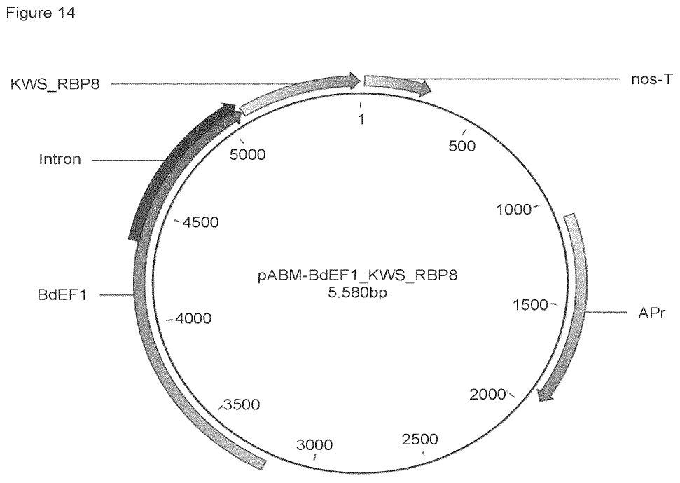

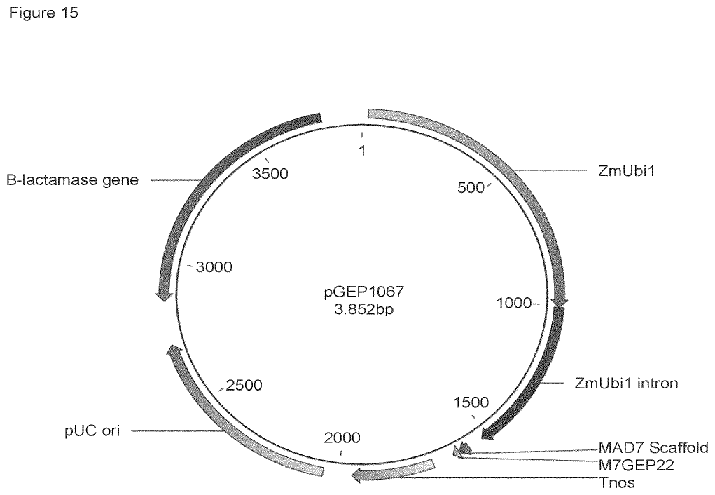

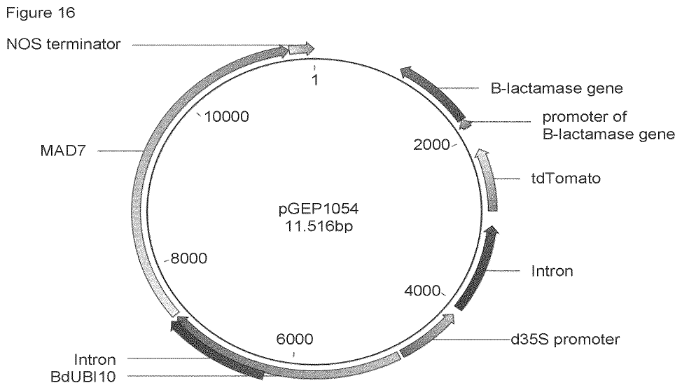

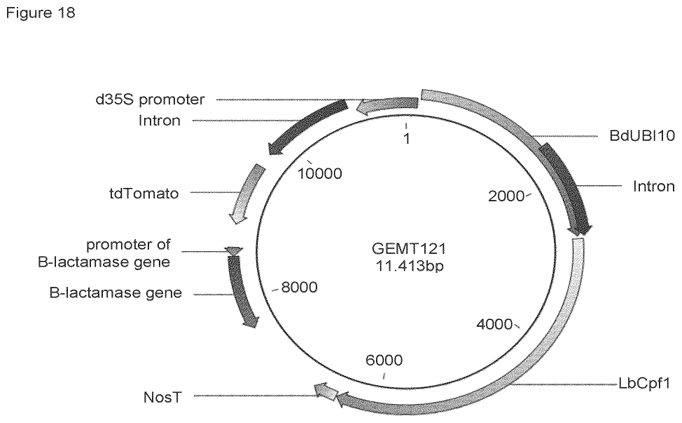

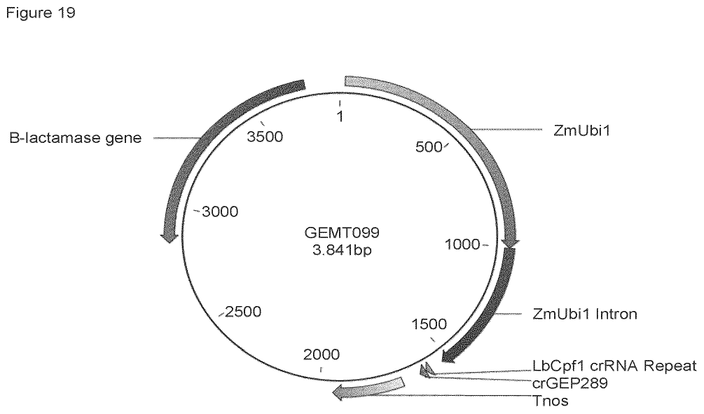

In the following, the term “RBG” means a regeneration booster gene, and “RBP” means a regeneration booster protein. As used herein, the term “RBP” may be used interchangeably to refer to a regeneration booster protein, but also to the cognate gene encoding this regeneration booster protein. Vice versa, a “RBG” may refer to a gene and the protein encoded by this gene accordingly.

| SEQ ID NO | Brief description |
|---|---|
| 1 | RBG1 CDS sequence |
| 2 | RBG2 CDS sequence |
| 3 | RBG3 CDS sequence |
| 4 | RBG4 CDS sequence |
| 5 | RBG5 CDS sequence |
| 6 | RBG6 CDS sequence |
| 7 | RBG7 CDS sequence |
| 8 | RBG8 CDS sequence |
| 9 | Zea mays, ZmPLT3-17207_CDS |
| 10 | Zea mays, ZmPLT5_CDS |
| 11 | Zea mays, ZmPLT7_CDS |
| 12 | Protein RBP1 |
| 13 | Protein RBP2 |
| 14 | Protein RBP3 |
| 15 | Protein RBP4 |
| 16 | Protein RBP5 |
| 17 | Protein RBP6 |
| 18 | Protein RBP7 |
| 19 | Protein RBP8 |
| 20 | Protein PLT3-17207-A188 |
| 21 | Protein ZmPLT5 |
| 22 | Protein ZmPLT7 |
| 23 | BdEF1_RBG1_expression_cassette |
| 24 | BdEF1_RBG2_expression_cassette |
| 25 | BdEF1_RBG3_expression_cassette |
| 26 | BdEF1_RBG4_expression_cassette |
| 27 | BdEF1_RBG5_expression_cassette |
| 28 | BdEF1_RBG6_expression_cassette |
| 29 | BdEF1_RBG7_expression_cassette |
| 30 | BdEF1_RBG8_expression_cassette |
| 31 | BdEF1_ZmPLT3_expression_cassette |
| 32 | BdEF1_ZmPLT5_expression_cassette |
| 33 | BdEF1_ZmPLT7_expression_cassette |
| 34 | pABM-BdEF1. This sequence represents the |
| | booster gene expression vector pABM-BdEF1. |
| | BdEF1 defines the strong constitutive EF1 promoter |
| | from Brachypodium. |
| 35 | pABM-BdEF1_RBG1 |
| 36 | pABM-BdEF1_RBG2 |
| 37 | pABM-BdEF1_RBG3 |
| 38 | pABM-BdEF1_RBG4 (FIG. 11) |
| 39 | pABM-BdEF1_RBG5 (FIG. 12) |
| 40 | pABM-BdEF1_RBG6 |
| 41 | pABM-BdEF1_RBG7 |
| 42 | pABM-BdEF1_RBG8 (FIG. 14) |
| 43 | pABM-BdEF1_ZmPLT3 |
| 44 | pABM-BdEF1_ZmPLT5 (FIG. 7) |
| 45 | pABM-BdEF1_ZmPLT7 |
| 46 | Plasmid pGEP837 MAD7 (FIG. 4) |
| 47 | Plasmid pGEP842 sgRNA m7GEP1 (FIG. 6) |
| 48 | Plasmid pGEP1054 Map and Plasmid tdTomato |
| | (FIG. 16) |
| 49 | Plasmid pGEP1067 sgRNA m7GEP22 (FIG. 15) |
| 50 | Construct GEMT121 encoding tdT as marker and |
| | LbCpf1 (FIG. 18) |
| 51 | Construct GEMT099 encoding sgRNA crGEP289 |
| | targeting wheat CPL3 (FIG. 19) |
| 52 | CDS of Triticum aestivum RKD4 |
| 53 | Protein TaRKD4 |

### Definitions

Unless defined otherwise, technical and scientific terms used herein have the same meaning as commonly understood by one of ordinary skill in the art to which this invention belongs.

As used in the context of the present application, the term “about” means +/−10% of the recited value, preferably +/−5% of the recited value. For example, about 100 nucleotides (nt) shall be understood as a value between 90 and 110 nt, preferably between 95 and 105 nt.

A “base editor” as used herein refers to a protein or a fragment thereof having the same catalytic activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor. Usually, base editors are thus used as molecular complex. Base editors, including, for example, CBEs (base editors mediating C to T conversion) and ABEs (adenine base editors mediating A to G conversion), are powerful tools to introduce direct and programmable mutations without the need for double-stranded cleavage (Komor et al., Nature, 2016, 533 (7603), 420-424; Gaudelli et al., Nature, 2017, 551, 464-471). In general, base editors are composed of at least one DNA targeting module and a catalytic domain that deaminates cytidine or adenine. All four transitions of DNA (A→T to G→C and C→G to T→A) are possible as long as the base editors can be guided to the target site. Originally developed for working in mammalian cell systems, both BEs and ABEs have been optimized and applied in plant cell systems. Efficient base editing has been shown in multiple plant species (Zong et al., Nature Biotechnology, vol. 25, no. 5, 2017, 438-440: Yan et al., Molecular Plant. vol. 11. 4, 2018, 631-634; Hua et al., Molecular Plant. vol. 11, 4. 2018, 627-630). Base editors have been used to introduce specific, directed substitutions in genomic sequences with known or predicted phenotypic effects in plants and animals. But they have not been used for directed mutagenesis targeting multiple sites within a genetic locus or several loci to identify novel or optimized traits.

A “CRISPR nuclease”, as used herein, is a specific form of a site-directed nuclease and refers to any nucleic acid guided nuclease which has been identified in a naturally occurring CRISPR system, which has subsequently been isolated from its natural context, and which preferably has been modified or combined into a recombinant construct of interest to be suitable as tool for targeted genome engineering. Any CRISPR nuclease can be used and optionally reprogrammed or additionally mutated to be suitable for the various embodiments according to the present invention as long as the original wild-type CRISPR nuclease provides for DNA recognition, i.e., binding properties. CRISPR nucleases also comprise mutants or catalytically active fragments or fusions of a naturally occurring CRISPR effector sequences, or the respective sequences encoding the same. A CRISPR nuclease may in particular also refer to a CRISPR nickase or even a nuclease-dead variant of a CRISPR polypeptide having endonucleolytic function in its natural environment. A variety of different CRISPR nucleases/systems and variants thereof are meanwhile known to the skilled person and include, inter alia. CRISPR/Cas systems, including CRISPR/Cas9 systems (EP2771468). CRISPR/Cpf1 systems (EP3009511B1). CRISPR/C2C2 systems. CRISPR/CasX systems. CRISPR/CasY systems. CRISPR/Cmr systems. CRISPR/MAD systems, including, for example CRISPR/MAD7 systems (WO2018236548A1) and CRISPR/MAD2 systems. CRISPR/CasZ systems and/or any combination. variant, or catalytically active fragment thereof. A nuclease may be a DNAse and/or an RNAse, in particular taking into consideration that certain CRISPR effector nucleases have RNA cleavage activity alone, or in addition to the DNA cleavage activity.

A “CRISPR system” is thus to be understood as a combination of a CRISPR nuclease or CRISPR effector, or a nickase or a nuclease-dead variant of said nuclease, or a functional active fragment or variant thereof together with the cognate guide RNA (or pegRNA or crRNA) guiding the relevant CRISPR nuclease.

As used herein, the terms “(regeneration) booster”. “booster gene”. “booster polypeptide”. “boost polypeptide”. “boost gene” and “boost factor”, refer to a protein/peptide(s), or a (poly) nucleic acid fragment encoding the protein/polypeptide, causing improved plant regeneration of transformed or gene edited plant cells, which may be particularly suitable for improving genome engineering, i.e., the regeneration of a modified plant cell after genome engineering. Such protein/polypeptide may increase the capability or ability of a plant cell, preferably derived from somatic tissue, embryonic tissue, callus tissue or protoplast, to regenerate in an entire plant, preferably a fertile plant. Thereby, they may regulate somatic embryo formation (somatic embryogenesis) and/or they may increase the proliferation rate of plant cells. The regeneration of transformed or gene edited plant cells may include the process of somatic embryogenesis, which is an artificial process in which a plant or embryo is derived from a single somatic cell or group of somatic cells. Somatic embryos are formed from plant cells that are not normally involved in the development of embryos, i.e, plant tissue like buds, leaves, shoots etc. Applications of this process may include: clonal propagation of genetically uniform plant material: elimination of viruses: provision of source tissue for genetic transformation: generation of whole plants from single cells, such as protoplasts; development of synthetic seed technology. Cells derived from competent source tissue may be cultured to form a callus. Further, the term “regeneration booster” may refer to any kind of chemical having a proliferative and/or regenerative effect when applied to a plant cell, tissue, organ, or whole plant in comparison to a no-treated control. The particular artificially created regeneration booster polypeptides according to the present invention may have the dual function of increasing plant regeneration as well as increasing desired genome modification and gene editing outcomes.

As used herein, a “flanking region”, is a region of the repair nucleic acid molecule having a nucleotide sequence which is homologous to the nucleotide sequence of the DNA region flanking (i.e. upstream or downstream) of the preselected site.

A “genome” as used herein is to be understood broadly and comprises any kind of genetic information (RNA/DNA) inside any compartment of a living cell. In the context of a “genome modification”, the term thus also includes artificially introduced genetic material, which may be transcribed and/or translated, inside a living cell, for example, an episomal plasmid or vector, or an artificial DNA integrated into a naturally occurring genome.

The term of “genome engineering” as used herein refers to all strategies and techniques for the genetic modification of any genetic information (DNA and RNA) or genome of a plant cell, comprising genome transformation, genome editing, but also including less site-specific techniques, including TILLING and the like. As such, “genome editing” (GE) more specifically refers to a special technique of genome engineering, wherein a targeted, specific modification of any genetic information or genome of a plant cell. As such, the terms comprise gene editing of regions encoding a gene or protein, but also the editing of regions other than gene encoding regions of a genome. It further comprises the editing or engineering of the nuclear (if present) as well as other genetic information of a plant cell, i.e., of intronic sequences, non-coding RNAs, miRNAs, sequences of regulatory elements like promoter, terminator, transcription activator binding sites, cis or trans acting elements. Furthermore, “genome engineering” also comprises an epigenetic editing or engineering, i.e., the targeted modification of, e.g., DNA methylation or histone modification, such as histone acetylation, histone methylation, histone ubiquitination, histone phosphorylation, histone sumoylation, histone ribosylation or histone citrullination, possibly causing heritable changes in gene expression.

A “genome modification system” as used herein refers to any DNA, RNA and/or amino acid sequence introduced into the cell, on a suitable vector and/or coated on particles and/or directly introduced, wherein the “genome modification system” causes the modification of the genome of the cell in which it has been introduced. A “genome editing system” more specifically refers to any DNA, RNA and/or amino acid sequence introduced into the cell, on a suitable vector and/or coated on particles and/or directly introduced, wherein the “genome editing system” comprises at least one component being, encoding, or assisting a site-directed nuclease, nickase or inactivated variant thereof in modifying and/or repairing a genomic target site.

A “genomic target sequence” as used herein refers to any part of the nuclear and/or organellar genome of a plant cell, whether encoding a gene/protein or not, which is the target of a site-directed genome editing or gene editing experiment.

A “plant material” as used herein refers to any material which can be obtained from a plant during any developmental stage. The plant material can be obtained either in planta or from an in vitro culture of the plant or a plant tissue or organ thereof. The term thus comprises plant cells, tissues and organs as well as developed plant structures as well as sub-cellular components like nucleic acids, polypeptides and all chemical plant substances or metabolites which can be found within a plant cell or compartment and/or which can be produced by the plant, or which can be obtained from an extract of any plant cell, tissue or a plant in any developmental stage. The term also comprises a derivative of the plant material, e.g., a protoplast, derived from at least one plant cell comprised by the plant material. The term therefore also comprises meristematic cells or a meristematic tissue of a plant.

The term “operatively linked”, “operably linked” or “functionally linked” specifically in the context of molecular constructs, for example plasmids or expression vectors, means that one element, for example, a regulatory element, or a first protein-encoding sequence, is linked in such a way with a further part so that the protein-encoding nucleotide sequence, i.e., is positioned in such a way relative to the protein-encoding nucleotide sequence on, for example, a nucleic acid molecule that an expression of the protein-encoding nucleotide sequence under the control of the regulatory element can take place in a living cell.

As used herein “a preselected site”, “predetermined site” or “predefined site” indicates a particular nucleotide sequence in the genome (e.g, the nuclear genome, or the organellar genome, including the mitochondrial or chloroplast genome) at which location it is desired to insert, replace and/or delete one or more nucleotides. The predetermined site is thus located in a “genomic target sequence/site” of interest and can be modified in a site-directed manner using a site- or sequence-specific genome editing system.

The terms “plant”, “plant organ”, or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example, animal cells, fungal cells or plant cells, can have any degree of ploidity, i.e, they may either be haploid, diploid, tetraploid, hexaploid or polyploid.

The term “plant parts” as used herein includes, but is not limited to, isolated and/or pre-treated plant parts, including organs and cells, including protoplasts, callus, leaves, stems, roots, root tips, anthers, pistils, seeds, grains, pericarps, embryos, pollen, sporocytes, ovules, male or female gametes or gametophytes, cotyledon, hypocotyl, spike, floret, awn, lemma, shoot, tissue, petiole, cells, and meristematic cells. A “Prime Editing system” as used herein refers to a system as disclosed in Anzalone et al. (2019). Search-and-replace genome editing without double-strand breaks (DSBs) or donor DNA. Nature. 1-1). Base editing as detailed above, does not cut the double-stranded DNA, but instead uses the CRISPR targeting machinery to shuttle an additional enzyme to a desired sequence, where it converts a single nucleotide into another. Many genetic traits in plants and certain susceptibility to diseases caused by plant pathogens are caused by a single nucleotide change, so base editing offers a powerful alternative for GE. But the method has intrinsic limitations, and is said to introduce off-target mutations which are generally not desired for high precision GE. In contrast. Prime Editing (PE) systems steer around the shortcomings of earlier CRISPR based GE techniques by heavily modifying the Cas9 protein and the guide RNA. The altered Cas9 only “nicks” a single strand of the double helix, instead of cutting both. The new guide RNA, called a pegRNA (prime editing extended guide RNA), contains an RNA template for a new DNA sequence, to be added to the genome at the target location. That requires a second protein, attached to Cas9 or a different CRISPR effector nuclease: a reverse transcriptase enzyme, which can make a new DNA strand from the RNA template and insert it at the nicked site. To this end, an additional level of specificity is introduced into the GE system in view of the fact that a further step of target specific nucleic acid::nucleic acid hybridization is required. This may significantly reduce off-target effects. Further, the PE system may significantly increase the targeting range of a respective GE system in view of the fact that BEs cannot cover all intended nucleotide transitions/mutations (C→A, C→G, G→C, G→T, A→C, A→T, T→A, and T→G) due to the very nature of the respective systems, and the transitions as supported by BEs may require DSBs in many cell types and organisms.

As used herein, a “regulatory sequence”, or “regulatory element” refers to nucleotide sequences which are not part of the protein-encoding nucleotide sequence, but mediate the expression of the protein-encoding nucleotide sequence. Regulatory elements include, for example, promoters, cis-regulatory elements, enhancers, introns or terminators. Depending on the type of regulatory element it is located on the nucleic acid molecule before (i.e., 5′ of) or after (i.e., 3′ of) the protein-encoding nucleotide sequence. Regulatory elements are functional in a living plant cell.

An “RNA-guided nuclease” is a site-specific nuclease, which requires an RNA molecule, i.e, a guide RNA, to recognize and cleave a specific target site, e.g, in genomic DNA or in RNA as target. The RNA-guided nuclease forms a nuclease complex together with the guide RNA and then recognizes and cleaves the target site in a sequence-dependent matter. RNA-guided nucleases can therefore be programmed to target a specific site by the design of the guide RNA sequence. The RNA-guided nucleases may be selected from a CRISPR/Cas system nuclease, including CRISPR/Cpf1 systems. CRISPR/C2C2 systems, CRISPR/CasX systems. CRISPR/CasY systems. CRISPR/Cmr systems. CRISPR/Cms systems. CRISPR/MAD7 systems. CRISPR/MAD2 systems and/or any combination, variant, or catalytically active fragment thereof. Such nucleases may leave blunt or staggered ends. Further included are nickase or nuclease-dead variants of an RNA-guided nuclease, which may be used in combination with a fusion protein, or protein complex, to alter and modify the functionality of such a fusion protein, for example, in a base editor or Prime Editor.

The terms “SDN-1”. “SDN-2”, and “SDN-3” as used herein are abbreviations for the platform technique “site-directed nuclease” 1, 2, or 3, respectively, as caused by any site directed nuclease of interest, including, for example. Meganucleases. Zinc-Finger Nucleases (ZFNs). Transcription Activator Like Effector Nucleases (TALENs), and CRISPR nucleases. SDN-1 produces a double-stranded or single-stranded break in the genome of a plant without the addition of foreign DNA. A “site-directed nuclease” is thus able to recognize and cut, optionally assisted by further molecules, a specific sequence in a genome or an isolate genomic sequence of interest. For SDN-2 and SDN-3, an exogenous nucleotide template is provided to the cell during the gene editing. For SDN-2, however, no recombinant foreign DNA is inserted into the genome of a target cell, but the endogenous repair process copies, for example, a mutation as present in the template to induce a (point) mutation. In contrast. SDN-3 mechanism use the introduced template during repair of the DNA break so that genetic material is introduced into the genomic material.

A “site-specific nuclease” herein refers to a nuclease or an active fragment thereof, which is capable to specifically recognize and cleave DNA at a certain location. This location is herein also referred to as a “target sequence”. Such nucleases typically produce a double-strand break (DSB), which is then repaired by non-homologous end-joining (NHEJ) or homologous recombination (HR). Site-specific nucleases include meganucleases, homing endonucleases, zinc finger nucleases, transcription activator-like nucleases and CRISPR nucleases, or variants including nickases or nuclease-dead variants thereof.

The terms “transformation”. “transfection”. “transformed”, and “transfected” are used interchangeably herein for any kind of introduction of a material, including a nucleic acid (DNA/RNA), amino acid, chemical, metabolite, nanoparticle, microparticle and the like into at least one cell of interest by any kind of physical (e.g., bombardment), chemical or biological (e.g., Agrobacterium) way of introducing the relevant at least one material.

The term “transgenic” as used according to the present disclosure refers to a plant, plant cell, tissue, organ or material which comprises a gene or a genetic construct, comprising a “transgene” that has been transferred into the plant, the plant cell, tissue organ or material by natural means or by means of transformation techniques from another organism. The term “transgene” comprises a nucleic acid sequence, including DNA or RNA, or an amino acid sequence, or a combination or mixture thereof. Therefore, the term “transgene” is not restricted to a sequence commonly identified as “gene”, i.e, a sequence encoding a protein. It can also refer, for example, to a non-protein encoding DNA or RNA sequence, or part of a sequence. Therefore, the term “transgenic” generally implies that the respective nucleic acid or amino acid sequence is not naturally present in the respective target cell, including a plant, plant cell, tissue, organ or material. The terms “transgene” or “transgenic” as used herein thus refer to a nucleic acid sequence or an amino acid sequence that is taken from the genome of one organism, or produced synthetically, and which is then introduced into another organism, in a transient or a stable way, by artificial techniques of molecular biology, genetics and the like.

As used herein, the term “transient” implies that the tools, including all kinds of nucleic acid (RNA and/or DNA) and polypeptide-based molecules optionally including chemical carrier molecules, are only temporarily introduced and/or expressed and afterwards degraded by the cell, whereas “stable” implies that at least one of the tools is integrated into the nuclear and/or organellar genome of the cell to be modified. “Transient expression” refers to the phenomenon where the transferred protein/polypeptide and/or nucleic acid fragment encoding the protein/polypeptide is expressed and/or active transiently in the cells, and turned off and/or degraded shortly with the cell growth. Transient expression thus also implies a stably integrated construct, for example, under the control of an inducible promoter as regulatory element, to regulate expression in a fine-tuned manner by switching expression on or off.

As used herein, “upstream” indicates a location on a nucleic acid molecule which is nearer to the 5′ end of said nucleic acid molecule. Likewise, the term “downstream” refers to a location on a nucleic acid molecule which is nearer to the 3′ end of said nucleic acid molecule. For avoidance of doubt, nucleic acid molecules and their sequences are typically represented in their 5′ to 3′ direction (left to right).

The terms “vector”, or “plasmid (vector)” refer to a construct comprising, inter alia, plasmids or (plasmid) vectors, cosmids, artificial yeast- or bacterial artificial chromosomes (YACs and BACs), phagemides, bacterial phage based vectors, Agrobacterium compatible vectors, an expression cassette, isolated single-stranded or double-stranded nucleic acid sequences, comprising sequences in linear or circular form, or amino acid sequences, viral vectors, viral replicons, including modified viruses, and a combination or a mixture thereof, for introduction or transformation, transfection or transduction into any eukaryotic cell, including a plant, plant cell, tissue, organ or material according to the present disclosure. A “nucleic acid vector, for instance, is a DNA or RNA molecule, which is used to deliver foreign genetic material to a cell, where it can be transcribed and optionally translated. Preferably, the vector is a plasmid comprising multiple cloning sites. The vector may further comprise a “unique cloning site” a cloning site that occurs only once in the vector and allows insertion of DNA sequences, e.g, a nucleic acid cassette or components thereof, by use of specific restriction enzymes. A “flexible insertion site” may be a multiple cloning site, which allows insertion of the components of the nucleic acid cassette according to the invention in an arrangement, which facilitates simultaneous transcription of the components and allows activation of the RNA activation unit. Whenever the present disclosure relates to the percentage of the homology or identity of nucleic acid or amino acid sequences to each other over the entire length of the sequences to be compared to each other, wherein these identity or homology values define those as obtained by using the EMBOSS Water Pairwise Sequence Alignments (nucleotide) programme nucleic acids or the EMBOSS Water Pairwise Sequence Alignments (protein) programme for amino acid sequences. Those tools provided by the European Molecular Biology Laboratory (EMBL) European Bioinformatics Institute (EBI) for local sequence alignments use a modified Smith-Waterman algorithm (see ebi.ac.uk/Tools/psa/and Smith, T. F. & Waterman, M. S. “Identification of common molecular subsequences” Journal of Molecular Biology, 1981 147 (1): 195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

### DETAILED DESCRIPTION

The present invention provides generally applicable genome and gene editing techniques relying on immature inflorescence meristem (IIM) cells as target material to be transformed/transfected providing better transformation and/or editing efficiencies in variety of relevant crop plants.

For all kinds of efficient plant transformations or transfections, the determination of the correct age and thus physiological status of the cells or material to be transformed is critical. Further, the decision on the target material to be transformed of interest may not only influence the susceptibility of the material for uptake of tools to be inserted, it may also significantly influence the outcome of a transformation. Efficiency of transformation or transfection, capability of regeneration after transformation and expression of molecular tools introduced, but, when it comes to gene editing, also factors like the epigenetic state of a material transformed may play an important role due to accessibility of a genome to be modified. Any off-target activity of the gene editing tools has to be avoided. Additionally, it is a very important factor that the desired modifications intended to be introduced during gene editing in a site-specific manner, but not necessarily the molecular tools transiently inserted, can be inherited to the offspring of a modified cell. For plant gene editing, this additionally implies that the modification is stable inherited in the relevant reproductive cells so that the resulting cells or organs, e.g., gametes, pollen, embryos etc., can be easily used for breeding new valuable plants. In view of these specific characteristics gene editing in usually rather complex plant genomes is still very often associated with severe problems and there is no convenient and straightforward way to transfer protocols gained in one system with a given gene editing machinery to another target plant and another genomic target region of interest to be modified.

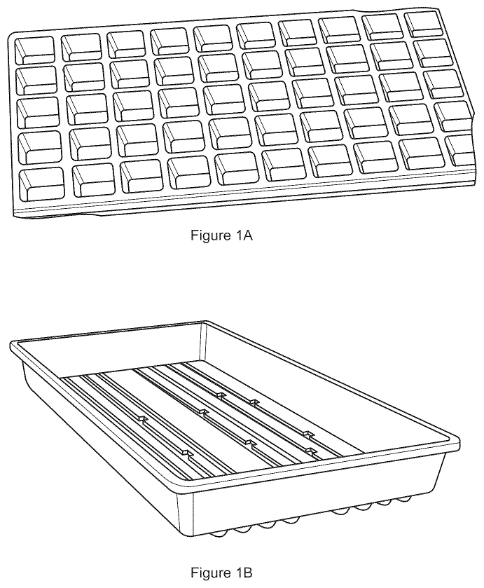

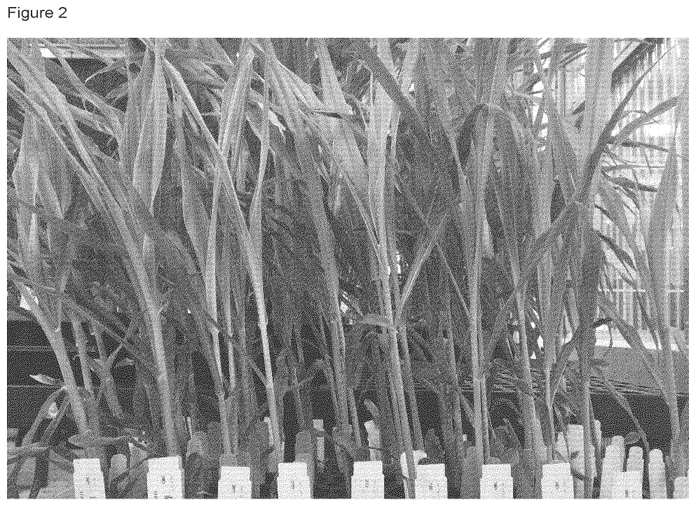

An inflorescence meristem is the modified shoot meristem that contains multipotent stem cells and is able to produce floral primordia, and eventually develops into an inflorescence, i.e., a cluster of flowers arranged on a main stem. The initiation of inflorescence meristem transition from shoot meristem is quite early in some cereal crops. For example, it takes about four weeks from seed planting to the IIM harvesting in maize (FIG. 2). The maize seeds can be planted and growing in a multiple-well tray (e.g. 50-well tray. FIG. 1) in any growth areas with simple lighting and temperature controls. Without the need for pollination and fertilization—processes which are very sensitive to environments and heavily depend on pollen and plant qualities—the IIM preparation can be performed greenhouse and growth season independent. Compared to using immature zygotic embryo, using IIM as the alternative donor explant further eliminates the problem of pollen contamination issues, and saves space, time, labour, and other resources for donor material preparation.

In one aspect, there is provided a method for plant genome modification, preferably for the targeted modification of at least one genomic target sequence, by obtaining a modification of at least one plant immature inflorescence meristem (IIM) cell, wherein the method comprises the following steps: (a) providing at least one immature inflorescence meristem cell: (b) introducing into the at least one immature inflorescence meristem cell: (i) at least one genome modification system, preferably a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same: (ii) optionally: at least one regeneration booster, or a sequence encoding the same, or a regeneration booster chemical, wherein steps (i) and (ii) take place simultaneously, or subsequently, for promoting plant cell proliferation and/or to assist in a targeted modification of at least one genomic target sequence: (iii) and, optionally at least one repair template, or a sequence encoding the same; and (c) cultivating the at least one immature inflorescence meristem cell under conditions allowing the expression and/or assembly of the at least one genome modification system, preferably the at least one genome editing system and optionally the at least one regeneration booster, and optionally of the at least one guide molecule and/or optionally of the at least one repair template; and (d) obtaining at least one modified immature inflorescence meristem cell, optionally by specifically screening for at least modified cell: or (e) obtaining at least one plant tissue, organ, plant or seed regenerated from the at least one modified cell; and (f) optionally: screening for at least one plant tissue, organ, plant or seed regenerated from the at least one modified cell in the T0 and/or T1 generation carrying a desired targeted modification.

To provide immature inflorescence meristem cells particularly suitable and accessible for effective particle bombardment and thus allowing for highly efficient genome editing, the present inventors tested immature inflorescence meristem cells from various cultivars of major crop plants. It was found that an explant comprising at least one immature inflorescence meristem cell could be favourably provided as cross-sectioned probe to better serve as an explant for biolistic transformation and to enhance subsequent regeneration to increase utilization efficiency. This finding is particularly important for some elite lines, including maize elite lines, where the initiation and development of axillary branches are significantly behind that of the center spike, so that the immature tassels therefore consist almost solely of center spike when harvested. The use of cross-section discs of immature center spike comprising at least one immature inflorescence meristem cell according to the present disclosure is thus an efficient solution for such genotypes in general to optimize regeneration and/or to achieve highly efficient genome editing in multiple locations simultaneously.

In certain embodiments, the at least one immature inflorescence meristem cell provided in step (a) in a method of the above first aspect thus may originate from a cross-section of a spike, or a structure being comparable to a spike with respect to developmental and overall morphological characteristics, wherein a spike comprises at least one immature inflorescence meristem cell, particularly wherein the at least one immature inflorescence meristem cell originates from a cross-section of a center spike of a crop plant of interest, for example, from a maize, wheat or barley plant. As it is known in the field of plant breeding and development, the spike is a structure that is usually formed from the inflorescence meristem through cell divisions to produce a main stem (rachis) and a spikelet meristem at each rachis node. Even though there are some morphological differences between spike and spikelet structure and development in different crop plants, the skilled person can determine the relevant developmental stages for a given crop plant of interest to obtain a cross-section of a spike, particularly of a center spike, as defined herein below.

In one embodiment, the introduction may preferably be at least one plant immature inflorescence meristem (IIM) cell may be mediated by biolistic bombardment.

In one aspect, there is provided a method of staging, i.e., defining a given developmental stage of a plant and the developing plant cells, including IIM cells, in a variety of crop plants.

Preferably, all exogenously provided elements or tools of a genome or gene editing system as well as optionally provided regeneration booster, or sequences encoding the same, and optionally provided repair template sequences are provided either simultaneously or subsequently, wherein the terms simultaneously and subsequently refers to the temporal order of introducing the relevant at least one tool, which may be introduced to be expressed transiently or in a stable manner, with the proviso that both simultaneous and subsequent introduction guarantee that one and the same IIM cell will comprise the relevant tools in an active and/or expressible manner. Ultimately, all genome modification or gene editing system elements are thus physically present in one IIM cell.

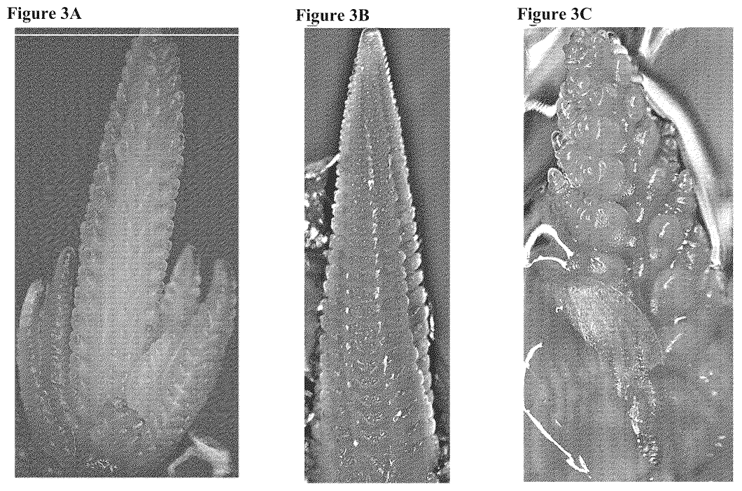

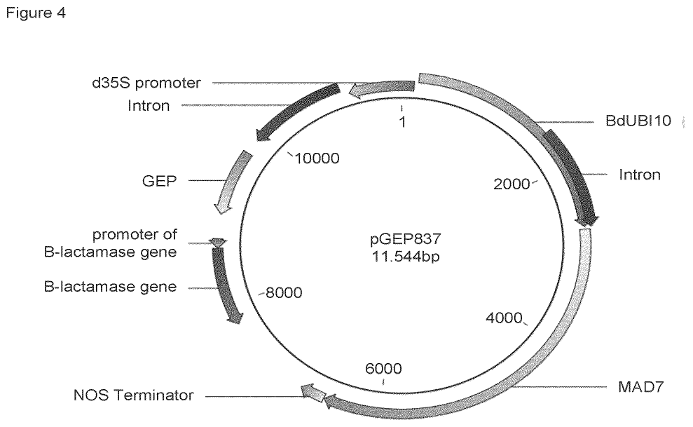

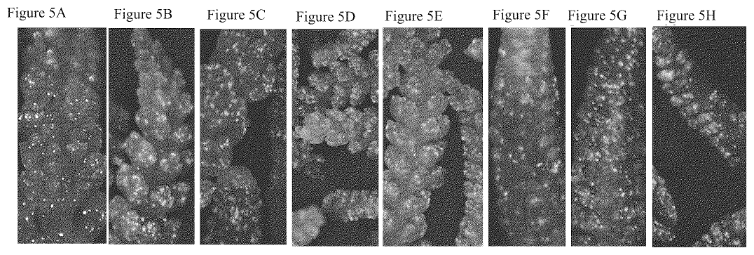

The immature inflorescence meristem (IIM) from Poaceae plants, including relevant crop plants, e.g., maize, wheat, rye, oat, barley, sorghum, rice, etc., is open at the stages when the floral bract primordia are underdeveloped (see FIG. 3). Therefore, the IIM cells are apt for genetic modification. The IIM cells are mitotically active and ready for regeneration without a need for cell identity reprogramming, and thus the IIM cells are highly regenerative and their regenerations are likely genotype-independent. The IIM cells are ideal recipients for transformation and regeneration. Moreover, the IIM cells are in reproduction phase, and developmentally close to meiosis, and thus the IIM cells may be in a HDR (Homology-Directed Repair)-friendly cell environment and suitable for HDR based genome editing. HDR may be preferable for different GE settings in view of the fact that targeted repair in the desired way can be achieved, in contrast to error-prone cellular repair processes.

Also for dicot plants, it could be demonstrated that staging of IIM cells and an efficient transformation of this specific cell type is possible according to the methods disclosed herein as, for example, shown in FIG. 21.

Based on the central findings of specifically choosing IIM cells for transformation, and the examples provided herein below giving guidance for the correct developmental staging to identify IIM tissues and cells in the developing inflorescence, the method of the present invention is applicable in any plant species, including monocot or dicot, of interest, preferably the methods may be performed in a plant being able to produce complex inflorescences (e.g., spike, spadix, capitulum or head) with sessile flowers (e.g., maize, rice, wheat, barley, sorghum, rye, sunflower, various kinds of berries).

In a further embodiment according to the various aspects of the present invention, at least one regeneration booster, or a sequence encoding the same, or a regeneration booster chemical is provided during genome or gene editing for promoting plant cell proliferation and/or to assist in a targeted modification of at least one genomic target sequence.

Certain regeneration booster sequences, usually representing transcription factors active during various stages of plant development and also known as morphogenic regulators in plants, are known for long, including the Wuschel (WUS) and babyboom (BBM) class of boosters (Mayer. K. F, et al. Role of WUSCHEL in regulating stem cell fate in the Arabidopsis shoot meristem. Cell 95, 805-815 (1998): Yadav. R. K, et al. WUSCHEL protein movement mediates stem cell homeostasis in the Arabidopsis shoot apex. Genes Dev 25, 2025-2030 (2011); Laux. T., Mayer, K. F . . . . Berger. J. & Jürgens. G. The WUSCHEL gene is required for shoot and floral meristem integrity in Arabidopsis. Development 122, 87-96 (1996); Leibfried. A, et al. WUSCHEL controls meristem function by direct regulation of cytokinin-inducible response regulators. Nature 438, 1172-1175 (2005): for BBM: Hofmann, A Breakthrough in Monocot Transformation Methods. The Plant Cell. Vol. 28:1989. September 2016). Others, including the RKD (including TaRKD4 disclosed herein as SEQ ID NOs: 52 and 53, or a variant, or a codon-optimized version thereof) and LEC family of transcription factors have been steadily emerging and are meanwhile known to the skilled person (Hofmann. A Breakthrough in Monocot Transformation Methods The Plant Cell. Vol. 28:1989. September 2016: New Insights into Somatic Embryogenesis: LEAFY COTYLEDON1. BABY BOOMI and WUSCHEL-RELATED HOMEOBOX4 Are Epigenetically Regulated in Coffea canephora. PLos one August 2013, vol. 8 (8), c72160; LEAFY COTYLEDON1-CASEIN KINASE I-TCP15-PHYTOCHROME INTERACTING FACTOR4 Network Regulates Somatic Embryogenesis by Regulating Auxin Homeostasis Plant Physiology_. December 2015. Vol. 169, pp. 2805-2821: A. Cagliari et al. New insights on the evolution of Leafy cotyledon1 (LEC1) type genes in vascular plants Genomics 103 (2014) 380-387. U.S. Pat. No. 6,825,397B1: U.S. Pat. No. 7,960,612B2. WO2016146552A1).

The Growth-Regulating Factor (GRF) family of transcription factors, which is specific to plants, is also known to the skilled person. At least nine GRF polypeptides have been identified in Arabidopsis thaliana (Kim et al. (2003) Plant J 36:94-104), and at least twelve in Oryza sativa (Choi et al. (2004) Plant Cell Physiol 45 (7): 897-904). The GRF polypeptides are characterized by the presence in their N-terminal half of at least two highly conserved domains, named after the most conserved amino acids within each domain: (i) a QLQ domain (InterPro accession IPR014978. PFAM accession PF08880), where the most conserved amino acids of the domain are Gln-Leu-Gln; and (ii) a WRC domain (InterPro accession IPR014977. PFAM accession PF08879), where the most conserved amino acids of the domain are Trp-Arg-Cys. The WRC domain further contains two distinctive structural features, namely, the WRC domain is enriched in basic amino acids Lys and Arg, and further comprises three Cys and one His residues in a conserved spacing (CX9CX10CX2H (SEQ ID NO: 54)), designated as the Effector of transcription (ET) domain (Ellerstrom ct al. (2005) Plant Molec Biol 59:663-681). The conserved spacing of cysteine and histidine residues in the ET domain is reminiscent of zinc finger (zinc-binding) proteins. In addition, a nuclear localisation signal (NLS) is usually comprised in the GRF polypeptide sequences.

Another class of potential regeneration boosters, vet not studied in detail for their function in artificial genome/gene editing, is the class of PLETHORS (PLT) transcription factors (Aida. M., et al. (2004). The PLETHORA genes mediate patterning of the Arabidopsis root stem cell niche. Cell 119:109-120; Mähönen. A. P., et al. (2014). PLETHORA gradient formation mechanism separates auxin responses. Nature 515:125-129). Organ formation in animals and plants relics on precise control of cell state transitions to turn stem cell daughters into fully differentiated cells. In plants, cells cannot rearrange due to shared cell walls. Thus, differentiation progression and the accompanying cell expansion must be tightly coordinated across tissues. PLETHORA (PLT) transcription factor gradients are unique in their ability to guide the progression of cell differentiation at different positions in the growing Arabidopsis thaliana root, which contrasts with well-described transcription factor gradients in animals specifying distinct cell fates within an essentially static context. To understand the output of the PLT gradient, we studied the gene set transcriptionally controlled by PLTs. Our work reveals how the PLT gradient can regulate cell state by region-specific induction of cell proliferation genes and repression of differentiation. Moreover. PLT targets include major patterning genes and autoregulatory feedback components, enforcing their role as master regulators of organ development (Santuari et al . . . 2016, DOI: doi.org/10.1105/tpc.16.00656). PLT also called AIL (AlNTEGUMENT-LIKE) genes, are members of the AP2 family of transcriptional regulators. Members of the AP2 family of transcription factors play important roles in cell proliferation and embryogenesis in plants (El Ouakfaoui. S, et al . . . (2010) Control of somatic embryogenesis and embryo development by AP2 transcription factors. PLANT MOLECULAR BIOLOGY 74 (4-5): 313-326.). PLT genes are expressed mainly in developing tissues of shoots and roots, and are required for stem cell homeostasis, cell division and regeneration, and for patterning of organ primordia. PLT family comprises an AP2 subclade of six members. Four PLT members. PLT1/AIL3 PLT2/. AIL4. PLT3/A/L6, and BBM/PLT4/AIL2, are expressed partly overlap in root apical meristem (RAM) and required for the expression of QC (quiescent center) markers at the correct position within the stem cell niche. These genes function redundantly to maintain cell division and prevent cell differentiation in root apical meristem. Three PLT gencs. PLT3/AIL6. PLT5/AIL5, and PLT7/AIL7, are expressed in shoot apical meristem (SAM), where they function redundantly in the positioning and outgrowth of lateral organs. PLT3, PLT5, and PLT7, regulate de novo shoot regeneration in Arabidopsis by controlling two distinct developmental events. PLT3. PLT5, and PLT7 required to maintain high levels of PINI expression at the periphery of the meristem and modulate local auxin production in the central region of the SAM which underlies phyllotactic transitions. Cumulative loss of function of these three genes causes the intermediate cell mass, callus, to be incompetent to form shoot progenitors, whereas induction of PLT5 or PLT7 can render shoot regeneration in a hormone-independent manner. PLT3, PLT5, PLT7 regulate and require the shoot-promoting factor CUP-SHAPED COTYLEDON2 (CUC2) to complete the shoot-formation program. PLT3, PLT5, and PLT7, are also expressed in lateral root founder cells, where they redundantly activate the expression of PLT1 and PLT2, and consequently regulate lateral root formation.

Regeneration boosters derived from naturally occurring transcription factors, as, for example, BBM or WUS, and variants thereof, may have the significant disadvantage that uncontrolled activity in a plant cell over a certain period of time will have deleterious effects on a plant cell. Therefore, the present inventors conducted a series of in silico work to create fully artificial regeneration booster proteins after a series of multiple sequence alignment, domain shuffling, truncations and codon optimization for various target plants. By focusing on core consensus motifs, it was an object to identify new variants of regeneration boosters not occurring in nature that are particularly suitable for us in methods for genome modifications and gene editing. Various gymnosperm sequences occurring in different species presently not considered as having a regeneration booster activity of described booster genes and proteins were particularly considered in the design process of the new booster sequences.

Based on this work, it was now found that specific regeneration boosters (cf. SEQ ID NOs: 1 to 8, 12 to 19), as well as certain modified regeneration boosters naturally acting as transcription factors (e.g., SEQ ID NOs: 9 to 11, 20 to 22) artificially created perform particularly well in combination with the methods disclosed herein, as they promote regeneration and additionally have the capacity to improve genome modification or gene editing efficiencies. Further, the artificially created and then stepwise selected and tested regeneration boosters do not show pleiotropic effects and are particularly suitable to be used during any kind of genome modification such as gene editing.

In one aspect, there is provided an isolated nucleic acid sequence encoding a regeneration booster polypeptide, wherein the nucleic acid sequence comprises a sequence selected from any one of SEQ ID NOs: 1 to 8, or a nucleic acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to the respective sequence of SEQ ID NOs: 1 to 8 with the proviso that the sequence encodes a regeneration booster with the same function as the respective reference sequence, or a nucleic acid sequence encoding a polypeptide comprises a sequence selected from any one of SEQ ID NOs: 12 to 19, or a sequence having at least 90%, 91%, 92%, 93%, 94%, 95%, 96%, 97%, 98%, 99% identity to the respective sequence of SEQ ID NOs: 12 to 19 with the proviso that the sequence has regeneration booster function as the respective reference sequence.

In a further aspect, there is provided a recombinant gene comprising an isolated nucleic acid sequence encoding a regeneration booster polypeptide, wherein the nucleic acid sequence comprises a sequence selected from any one of SEQ ID NOs: 1 to 8, or a nucleic acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to the respective sequence of SEQ ID NOs: 1 to 8 with the proviso that the sequence encodes a regeneration booster with the same function as the respective reference sequence, or a nucleic acid sequence encoding a polypeptide comprises a sequence selected from any one of SEQ ID NOs: 12 to 19, or a sequence having at least 90%, 91%, 92%, 93%, 94%, 95%, 96%, 97%, 98%, 99% identity to the respective sequence of SEQ ID NOs: 12 to 19 with the proviso that the sequence has regeneration booster function as the respective reference sequence.

In one embodiment, the recombinant gene may comprise at least one regulatory element as detailed below. In view of the fact that the regeneration booster genes disclosed herein are fully artificial, there is no classical “natural” regulatory element, e.g., a promoter, to be used. Therefore, the choice of at least one suitable regulatory element will be guided by the question of the host cell of interest and/or spatio-temporal expression patterns of interest, so that the optimum regulatory elements can be chosen to achieve a specific expression of the at least one regeneration booster gene of interest.

In one embodiment, wherein more than one regeneration booster gene are used, different promoters may be chosen, for example, the promoters having different activities so that the at least two genes can be expressed in a defined and controllable manner to have a stronger expression of a first regeneration booster protein/polypeptide (RBP) and a weaker expression of a second RBP, where a differential expression pattern may be desired.

In one aspect, there is provided isolated regeneration booster polypeptide wherein the polypeptide comprises a sequence selected from any one of SEQ ID NOs: 12 to 19, or a sequence having at least 90%, 91%, 92%, 93%, 94%, 95%, 96%, 97%, 98%, 99% identity to the respective sequence of SEQ ID NOs: 12 to 19 with the proviso that the sequence has regeneration booster function as the respective reference sequence.

In yet another aspect, there is provided an expression cassette or an expression construct comprising a sequence encoding a regeneration booster polypeptide comprising a nucleic acid sequence selected from any one of SEQ ID NOs: 12 to 19, or a nucleic acid sequence having at least 90%, 91%, 92%, 93%, 94%, 95%, 96%, 97%, 98%, 99% identity to the respective sequence of SEQ ID NOs: 12 to 19 with the proviso that the sequence has regeneration booster function as the respective reference sequence, or a nucleic acid sequence encoding a polypeptide comprises a sequence selected from any one of SEQ ID NOs: 12 to 19, or a sequence having at least 90%, 91%, 92%, 93%, 94%, 95%, 96%, 97%, 98%, 99% identity to the respective sequence of SEQ ID NOs: 12 to 19 with the proviso that the sequence has regeneration booster function as the respective reference sequence. In certain embodiments, the expression cassette or the expression construct may be selected from any one of SEQ ID NOs: 23 to 30, or 35 to 42, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to the respective sequence′.

In still another aspect, there is provided a plant cell comprising a recombinant gene comprising an nucleic acid sequence encoding a regeneration booster polypeptide, wherein the nucleic acid sequence comprises a sequence selected from any one of SEQ ID NOs: 1 to 8, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to the respective sequence of SEQ ID NOs: 1 to 8 with the proviso that the sequence encodes a regeneration booster with the same function as the respective reference sequence, or comprising an expression cassette or an expression construct comprising a sequence encoding a regeneration booster polypeptide comprising a sequence selected from any one of SEQ ID NOs: 12 to 19, or a sequence having at least 90%, 91%, 92%, 93%, 94%, 95%, 96%, 97%, 98%, 99% identity to the respective sequence of SEQ ID NOs: 12 to 19 with the proviso that the sequence has regeneration booster function as the respective reference sequence.

In one aspect, there is provided a plant tissue, organ, whole plant, or a part thereof or a seed of a monocot or dicot plant of interest comprising the plant cell comprising the recombinant gene or comprising the expression cassette or the expression construct as defined above.

Based on the effects of the new regeneration boosters, or the new combination of regeneration boosters, as disclosed herein, it is possible to transform or transfect even recalcitrant plants/plant genotypes, or cells, tissues or organs comprised by, or obtained from a recalcitrant plant/plant genotype, i.e., those plants/plant genotypes usually known to be very hard to transform or transfect with exogenous material and/or which are known to have a weak regeneration and/or developmental activity. As detailed in Example 8 below, the various methods as disclosed herein are particularly suitable for modifying, i.e., transforming or transfecting, recalcitrant plants/plant genotypes or plant cells.

In one embodiment, the regeneration booster comprises at least one RBP, or an regeneration booster gene (RBG) sequence encoding the RBP, wherein the at least one of an RBP sequence is individually selected from any one of SEQ ID NOs: 13, or 15 to 19, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the RBP is encoded by at least one RBG sequence, wherein the at least one of an RBP sequence is individually selected from any one of SEQ ID NOs: 2, or 4 to 8, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a cognate codon-optimized sequence.

Additionally, the regeneration booster sequences, or the sequences encoding the same, according to SEQ ID NOs: 1 to 8 and 12 to 19 were studied in detail to identify suitable combinations of regeneration boosters to be provided during genome or gene editing to achieve even synergistic activities in promoting regeneration, e.g., during any kind of plant transformation, and/or to optimize gene editing frequencies.

In one embodiment, the regeneration booster comprises at least one RBP and at least one PLT encoding sequence, wherein the RBP and the PLT regeneration booster sequence is individually selected from any one of SEQ ID NOs: 12 to 22, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or a catalytically active fragment thereof, or wherein the at least one regeneration booster sequence is encoded by a sequence individually selected from any one of SEQ ID NOs: 1 to 11, or a sequence having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, provided that the sequence encodes the respective regeneration booster according to SEQ ID NOs: 12 to 22 or a catalytically active fragment thereof.

In another embodiment of the various methods disclosed herein, the at least one further regeneration booster is introduced, wherein the further regeneration booster, or the sequence encoding the same is selected from BBM, WUS, WOX, (Ta) RKD4, growth-regulating factors (GRFs), LEC, or a variant thereof.

In yet another embodiment of the various methods disclosed herein, the regeneration booster comprises at least one first RBG or PLT sequence, or the sequence encoding the same, preferably at least one RBG sequence, or the sequence encoding the same, and wherein the regeneration booster further comprises: (i) at least one further RBG and/or PLT sequence, or the sequence encoding the same, or a variant thereof, and/or (ii) at least one BBM sequence, or the sequence encoding the same, or a variant thereof, and/or (iii) at least one WOX sequence, including WUS1, WUS2, or WOX5, or the sequence encoding the same, or a variant thereof, and/or (iv) at least one RKD4 sequence, including wheat RKD4, or the sequence encoding the same, or a variant thereof, and/or (v) at least one GFR sequence, including GRFI or GRF5, or the sequence encoding the same, or a variant thereof, and/or (vi) at least one LEC sequence, including LEC1 and LEC2, or the sequence encoding the same, or a variant thereof as at least one second regeneration booster, or sequence encoding the same, different to the first regeneration booster.

In preferred embodiments according to the methods disclosed herein, at least the first, or the exclusive, regeneration booster used, or the sequence encoding the same, is a RBP, or the respective RBG sequence, according to SEQ ID NOs: 1 to 8 and 12 to 19, respectively, or a sequence having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

In embodiments, where a single regeneration booster is used, the regeneration booster may be selected from SEQ ID NOs: 13, and 15 to 19, or the sequences encoding the same, or from SEQ ID NOs: 20 to 22, or the sequences encoding the same, or a sequence having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

In embodiments, where a combination of two regeneration boosters is used, these combinations may be selected from (i) a specific combination of RBP8, RBP7, RBP5, RBP2, RBP6, RBP4, or RBP3 as first booster with either one of PLT3, PLT5, or PLT7 (for reference regarding abbreviations and corresponding SEQ ID NOs, see Description of Sequences above): (ii) RBP8, RBP7, RBP5, RBP2, RBP6, RBP4, or RBP3 as first booster and a suitable BBM, e.g., ZmBBM: (iii) PLT3, PLT5, or PLT7 as first regeneration booster and WUS1, or WUS2, e.g. ZmWUS1 and WUS2: (iv) RBP8, RBP7, RBP5, RBP2, RBP6, RBP4, or RBP3 as first booster and RKD4, preferably TaRKD4 (from Triticum aestivum L., cf. SEQ ID NOs: 52 and 53); (v) PLT3, PLT5, or PLT7 as first regeneration booster and RKD4, preferably TaRKD4: (vi) RBP8, RBP7. RBP5, RBP2, RBP6, RBP4, or RBP3 as first booster and LEC1 or LEC2, for example. ZmLEC1 or ZmLEC2, as second booster: (vii) PLT3, PLT5, or PLT7 as first regeneration booster and a LEC1 or LEC2 as second booster: (viii) RBP8, RBP7, RBP5, RBP2, RBP6, RBP4, or RBP3 as first booster and a GRF, for example GRF5, as second booster: (ix) PLT3, PLT5, or PLT7 as first regeneration booster and a GRF as second booster: (x) RKD4, for example. TaRKD4 as first regeneration booster and a GRF family member as second booster: or (xi) a GRF family member as first regeneration booster and LEC1 or LEC2, or the corresponding sequences encoding the same, or a sequence having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

According to the various embodiments and aspects disclosed herein, it may be preferable to use a naturally occurring regeneration booster in addition to an artificial RBP according to the present invention, wherein the naturally occurring regeneration booster, e.g., BBM. WUS1/2. LEC1/2. GRF, or a PLT may be derived from a target plant to be transformed, or from a closely related species. For monocot plant modifications, for example, a booster protein with monocot origin (e.g., from Zea mays (Zm)) may be preferred, whereas for dicot plant modifications, a booster protein with dicot origin (e.g., originating from Arabidopsis thaliana (At), or Brassica napus (Bn)) may be preferred. The relevant booster sequences can be easily identified by sequence searches within the published genome data. Notably, regeneration boosters from one plant species may show a certain cross-species applicability so that, for example, a wheat-derived booster gene may be used in Zea mays, and vice versa, or a Arabidopsis- or Brachypodium-derived booster gene may be used in Helianthus, and vice versa. A PLT, WUS, WOX, BBM, LEC, RKD4, or GRF sequence as used herein, or a protein with a comparable regeneration booster function, may thus be derived from any plant species harbouring a corresponding gene encoding the respective booster in its genome.

In embodiments, where a combination of three regeneration boosters is used, these combinations may be selected from (i) RBP8, RBP7, RBP5, RBP2, RBP6, RBP4, or RBP3 as first booster. PLT3, PLT5, or PLT7, or a BBM as second regeneration booster, and RKD4 as third regeneration booster: (ii) PLT3, PLT5, or PLT7, or a BBM as first regeneration booster. RKD4 as second regeneration booster, and WUS1 or WUS2 as third regeneration booster; (iii) RBP8, RBP7, RBP5, RBP2, RBP6, RBP4, or RBP3 as first booster, a PLT3, PLT5, or PLT7, or a BBM as second regeneration booster, and a LEC1 or LEC2 as third regeneration booster: (iv) ZmPLT3. ZmPLT5, or ZmPLT7 as first regeneration booster. ZmLEC1 or ZmLEC2 as second regeneration booster, and a WUS1 or WUS2 as third regeneration booster: (v) RBP8, RBP7, RBP5, RBP2. RBP6. RBP4, or RBP3 as first regeneration booster, an RKD4 as second regeneration booster and a LEC1 or a LEC2 as third regeneration booster: (vi) a PLT3, PLT5, or PLT7 as first regeneration booster, a RKD4 as second regeneration booster, and a LEC1 or LEC2 as third regeneration booster: (vii) RBP8, RBP7. RBP5, RBP2, RBP6, RBP4, or RBP3 as first regeneration booster, a GRF as second regeneration booster, and a PLT3, PLT5, PLT7 or a BBM as third regeneration booster: (viii) a PLT3, PLT5, or PLT7 as first regeneration booster, a GRF as second regeneration booster, and a WUS1 or WUS2 as third regeneration booster: (ix) RBP8, RBP7, RBP5, RBP2, RBP6, RBP4, or RBP3 as first regeneration booster, a GRF as second regeneration booster, and a RKD4 as third regeneration booster; or (x) a PLT3, PLT5, or PLT7 as first regeneration booster, a GRF as second regeneration booster, and a RKD4 as third regeneration booster, or the corresponding sequences encoding the same, or a sequence having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

It was found that the use of at least one regeneration booster, preferably a booster, or a specific combination of boosters as detailed above, in connection with the methods of the present invention can have a dual effect: either the improvement of any kind of transient or stable transformation in which a transgene is ectopically expressed, or in the specific setting of gene editing relying on the use of at least one site-specific nuclease, wherein the editing efficiency is improved by the presence of at least one booster as disclosed herein.

A “regeneration booster” as used herein may not only refer to a protein, or a sequence encoding the same, having plant proliferative activity, as defined above. A “regeneration booster” may also be a chemical added during genome modification of an IIM cell, or tissue or plant comprising the same.

In one embodiment, the regeneration booster may thus be a chemical selected from MgCl, or MgSO+, for example in a range from about 1 to 100 mM, preferably in a range from about 10 to 20 mM, spermidine in a range from about 0.1-1 mM, preferably in a range from about 0.1-0.5 mM, TSA (trichostatin A), and TSA-like chemicals.

The use of at least one regeneration booster in an artificial and controlled context according to the methods disclosed herein thus has the effect of promoting plant cell proliferation. This effect is highly favourable for any kind of plant genome modification, as it promotes cell regeneration after introducing any plasmid or chemical into the at least one plant cell via transformation and/or transfection, as these interventions necessarily always cause stress to a plant cell.

Additionally, or alternatively, the at least one regeneration booster according to the methods disclosed herein may have a specific effect in enhancing plant genome editing efficiency. In particular, this kind of intervention caused by at least one site-specific nuclease, nickase or a variant thereof, causes a certain repair and stress response in a plant. The presence of at least one regeneration booster can thus also improve the efficiency of genome modification or gene editing by increasing the regeneration rate of a plant cell after a modification of the plant genome.

In one embodiment, at least one regeneration booster, or a sequence encoding the same, or a regeneration booster chemical, can be provided simultaneously with other tools to be inserted, namely the at least one genome modification system, preferably the genome editing system to reduce the number of transformation/transfection acts potentially stressful for a cell. For certain cells sensitive to transformation/transfection, regeneration booster chemicals may thus represent a suitable option, which may be provided before, simultaneously with, or soon after transforming/transfecting further genome or gene editing tools to reduce the cellular stress and to increase transformation and/or editing efficiency by stabilizing a cell and thus by reducing potentially harmful cellular stress responses.

In another embodiment, the at least one genome modification system, preferably the genome editing system and the at least one regeneration booster, or the sequence encoding the same, may be provided subsequently or sequentially. By separating the introduction steps, the editing construct DNA integration of the site-directed nuclease, nickase or an inactivated nuclease encoding sequence can be avoided, where transient outcomes are of interest.

In certain embodiments, it is favourable that the at least one regeneration booster is active in a cell before further tools are introduced to put the cell into a state of low cellular stress before performing genome or gene editing.

For any simultaneous or subsequent introduction of at least one regeneration booster, the regeneration booster and the optional further genome modification or genome editing system should be active, i.e., present in the active protein and/or RNA stage, in one and the same cell to be modified, preferably in the nucleus of the cell, or in an organelle comprising genomic DNA to be modified.

Without a reasonable strategy to regenerate (effectively) transformed plant cells, there is little impact of a GE protocol. IIM cells, if transformed in the correct developmental stage following the protocols provided herein, have the intrinsic capacity to be regenerated in various ways to plant tissues, organs, whole plants and seeds in a flexible manner in addition to the fact that these cells can be modified in a targeted manner according to the methods disclosed herein.

In one aspect, there is provided a method of producing a haploid or doubled haploid plant cell, tissue, organ, plant, or seed.

The generation and use of haploids is one of the most powerful biotechnological means to improve cultivated plants. The advantage of haploids for breeders is that homozygosity can be achieved already in the first generation after dihaploidization, creating doubled haploid plants, without the need of laborious backcrossing steps to obtain a high degree of homozygosity. Furthermore, the value of haploids in plant research and breeding lies in the fact that the founder cells of doubled haploids are products of meiosis, so that resultant populations constitute pools of diverse recombinant and at the same time genetically fixed individuals. The generation of doubled haploids thus provides not only perfectly useful genetic variability to select from with regard to crop improvement, but is also a valuable means to produce mapping populations, recombinant inbreds as well as instantly homozygous mutants and transgenic lines.

Haploid plants can be obtained by interspecific crosses, in which one parental genome is eliminated after fertilization. It was shown that genome elimination after fertilization could be induced by modifying a centromere protein, the centromere-specific histone CENH3 in Arabidopsis thaliana (Ravi and Chan, Haploid plants produced by centromere-mediated genome elimination, Nature, Vol. 464, 2010, 615-619). With the modified haploid inducer lines, haploidization occurred in the progeny when a haploid inducer plant was crossed with a wild type plant. Interestingly, the haploid inducer line was stable upon selfing, suggesting that a competition between modified and wild type centromere in the developing hybrid embryo results in centromere inactivation of the inducer parent and consequently in uniparental chromosome elimination.

In one embodiment, the methods of the present invention thus comprise the generation of at least one haploid cell, tissue or organ having activity of a haploid inducer, preferably wherein the haploid cell, tissue or organ comprises a callus tissue, male gametophyte or microspore. In this embodiment, the methods as disclosed herein may comprise the introduction of a nucleotide or amino acid sequence encoding or being a sequence allowing the generation of a haploid inducer cell, for example a sequence encoding a KINETOCHORE NULL2 (KNL2) protein comprising a SANTA domain, wherein the nucleotide sequence comprises at least one mutation causing in the SANTA domain an alteration of the amino acid sequence of the KNL2 protein and said alteration confers the activity of a haploid inducer (as disclosed in EP 3 159 413 A1) in a method for plant genome modification, preferably for the targeted modification of at least one genomic target sequence, for obtaining a modification of at least one plant immature inflorescence meristem cell. In this embodiment, the at least one genome modification system does not comprise a genome editing system, but the sequence allowing the generation of a haploid inducer line, which is introduced into a plant cell to be modified stably or transiently, in a constitutive or inducible manner.

In another embodiment, the modified cell according to the methods of the present invention is a haploid cell, wherein the haploid cell is generated by introducing a genome editing system into at least one cell, preferably an IIM cell, to be modified, wherein the genome editing system is capable of introducing at least one mutation into the genomic target sequence of interest resulting in a cell having haploid inducer activity.

In yet a further aspect, there is provided a method for producing a haploid or doubled haploid plant cell, tissue, organ, plant, or seed, wherein the method comprises providing at least one regeneration booster, or a specific combination of regeneration boosters, or the sequence(s) encoding the same, to at least one cell to be modified, wherein the at least one cell is preferably a haploid cell, for example, a gametophyte or microspore. These inherently haploid cells of plants produced during the reproduction cycle have the intrinsic characteristic of being very inert to any kind of chromosome doubling and transformation. The methods as disclosed herein can thus be favourably used to introduce or apply at least one regeneration booster, or a sequence encoding the same, or a regeneration booster chemical for promoting the regenerative capacity of a haploid plant cell to increase the capacity of the haploid cell for a conversion during chromosome doubling, as the doubled haploid material is of particular interest for breeding and ultimately cultivating plants. The methods as disclosed herein thus overcome the difficulties in handling haploid plants cells and tissues, including callus tissue, as the frequency of induced and/or spontaneous chromosome doubling can be increased by providing at least one booster sequence, or preferably a specific combination of booster sequences, as disclosed herein.

Various methods for doubling chromosomes in plant biotechnology are available to the skilled person for various cultivars. In one embodiment, chromosome doubling can be achieved by using colchicine treatment. Other chemicals for chromosome doubling, are available for use according to the methods disclosed herein, wherein these chemicals may be selected from antimicrotubule herbicides, including amiprophosmethyl (APM), pronamide, oryzalin, and trifluralin, which are all known for their chromosome doubling activity.

In certain embodiments, there is provided a method comprising a regeneration step, wherein the regeneration may be performed either by direct meristem organogenesis, i.e., by directly obtaining a viable plant cell, tissue, organ, plant or seed modified as detailed above, or wherein the regeneration may be performed indirectly, i.e., via an additional cell culture step proceeding through callus organogenesis. Further provided are suitable methods for regenerating at least one immature inflorescence meristem cell, into which at least one genome or gene editing tool has been inserted according to the methods for plant genome modification disclosed herein either by direct meristem organogenesis, or by indirect callus embryogenesis or organogenesis.

The fact that the regeneration can be performed either directly or indirectly, as detailed below in various Examples, is a huge advantage as it offers several options and flexible strategies, depending on a target plant of interest, to obtain viable plant material from at least one treated IIM cell for various relevant crop plants and allows rapid progress in breeding programs, when combining them with the methods disclosed herein.

In one embodiment, the at least one genome modification system, preferably the at least one genome editing system and optionally the at least one regeneration booster, or the sequences encoding the same, are introduced into the cell by transformation or transfection mediated by biolistic bombardment, Agrobacterium-mediated transformation, micro- or nanoparticle delivery, or by chemical transfection, or a combination thereof, preferably wherein the at least one genome modification system, preferably the at least one genome editing system, and optionally the at least one regeneration booster are introduced by biolistic bombardment.

Particle or biolistic bombardment may be a preferred strategy according to the methods disclosed herein, as it allows the direct and targeted introduction of exogenous nucleic acid and/or amino acid material in a precise manner not relying on the biological spread and expression of biological transformation tools, including Agrobacterium.

In certain embodiments, the biolistic bombardment comprises a step of osmotic treatment before and/or after bombardment. Osmotic treatment can be highly suitable to enhance the transformation/transfection capacity of a cell before bombardment. Further, it can increase the transformation/transfection efficiency after bombardment. Various osmotic treatment protocols are disclosed below, and further cell-type specific protocols are available to the skilled person in the field of plant biotechnology.

As introduced above, IIM cells, due to their state of development and the physical accessibility to transformation/transfection techniques, thus represent a valuable target cell type for efficient methods for plant genome modification. To increase the genome or gene editing efficiency, the methods can not only rely on the introduction of a genome modification system, i.e., any vector or pre-assembled complex comprising nucleic acid and/or amino acid material, the methods as disclosed herein may be particularly effective in case at least one specific regeneration booster as disclosed herein is provided (introduced or, for chemicals, applied) in parallel to alleviate stress responses in a cell and to allow rapid recovery and regeneration after a manipulation.

Additionally, in certain embodiments, the methods as disclosed herein for the targeted modification of the plant genome of at least one IIM cell can comprise the introduction of a genome modification system or a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same, optionally together with the introduction of at least one repair template, or a sequence encoding the same.

The at least one genome editing system may be provided with or without the provision of at least one regeneration booster in view of the fact that IIM cells as disclosed herein as new targets for efficient plant genome modification of various relevant crop plants as such represent valuable and easily accessible target structures with the capacity to regenerate into viable plant cells, tissues, organs, whole plants or seeds thereof.

Genome modification and site-directed genome editing efficiency is largely controlled by host cell statuses. Cells undergoing rapid cell-division, like those in plant meristems, in particular IIM cells studied herein, were shown to be the most suitable recipients for genome engineering according to the methods established herein. It was further shown that promoting cell division by providing suitable regeneration boosters and combinations thereof increases DNA integration or modification during DNA replication and division process, and thus significantly increases genome editing efficiency.

In certain embodiments, at least one genome modification system, preferably a genome editing system may be provided together with, i.e., simultaneously, or subsequently, but to one and the same target cell, the at least one regeneration booster, or regeneration booster chemical. This strategy does not only profit from the general effects of regeneration boosters on the regenerative capacity of a plant cell, the combined use may also increase genome editing efficiency in a synergistic way. Any kind of site-directed genome editing leaves a single- or double-strand break and/or modified a certain base in a genomic target sequence of interest. This manipulation initiates stress and cellular repair responses hampering a generally high genome editing efficiency. The combined introduction of at least one genome editing system and at least one regeneration booster, or a regeneration booster chemical, can thus dramatically increase the frequency of site-directed positive (i.e., desired) genome editing events detectable throughout a high proportion of relevant target cells transformed/transfected.

In certain embodiments, where at least one genome editing system is introduced according to the methods disclosed herein, the methods include the introduction of at least one site-directed nuclease, nickase or an inactivated nuclease, or a sequence encoding the same, wherein the site-directed nuclease, nickase or an inactivated nuclease may be selected from the group consisting of a CRISPR nuclease or a CRISPR system, including a CRISPR/Cas system, preferably from a CRISPR/MAD7 system, a CRISPR/Cfpl system, a CRISPR/MAD2 system, a CRISPR/Cas9 system, a CRISPR/CasX system, a CRISPR/CasY system, a CRISPR/Cas 13 system, or a CRISPR/Csm system, a zinc finger nuclease system, a transcription activator-like nuclease system, or a meganuclease system, or any combination, variant, or catalytically active fragment thereof.

In certain embodiments, wherein at least one genome editing system is introduced, the at least one genome editing system may further comprise at least one reverse transcriptase and/or at least one cytidine or adenine deaminase, preferably wherein the at least one cytidine or adenine deaminase is independently selected from an apolipoprotein B mRNA-editing complex (APOBEC) family deaminase, preferably a rat-derived APOBEC, an activation-induced cytidine deaminase (AID), an ACFI/ASE deaminase, an ADAT family deaminase, an ADAR2 deaminase, or a PmCDA deaminase, a TadA derived deaminase, and/or a transposon, or a sequence encoding the aforementioned at least one enzyme, or any combination, variant, or catalytically active fragment thereof.

A variety of suitable genome editing systems that can be employed according to the methods of the present invention, is available to the skilled person and can be easily adapted for use in the methods used herein.

In embodiments, wherein the site-directed nuclease or variant thereof is a nucleic acid-guided site-directed nuclease, the at least one genome editing system additionally includes at least one guide molecule, or a sequence encoding the same. The “guide molecule” or “guide nucleic acid sequence” (usually called and abbreviated as guide RNA, crRNA, crRNA+tracrRNA, gRNA, sgRNA, depending on the corresponding CRISPR system representing a prototypic nucleic acid-guided site-directed nuclease system), which recognizes a target sequence to be cut by the nuclease. The at least one “guide nucleic acid sequence” or “guide molecule” comprises a “scaffold region” and a “target region”. The “scaffold region” is a sequence, to which the nucleic acid guided nuclease binds to form a targetable nuclease complex. The scaffold region may comprise direct repeats, which are recognized and processed by the nucleic acid guided nuclease to provide mature crRNA. A pegRNAs may comprise a further region within the guide molecule, the so-called “primer-binding site”. The “target region” defines the complementarity to the target site, which is intended to be cleaved. A crRNA as used herein may thus be used interchangeably herein with the term guide RNA in case it unifies the effects of meanwhile well-established CRISPR nuclease guide RNA functionalities. Certain CRISPR nucleases, e.g., Cas9, may be used by providing two individual guide nucleic acid sequences in the form of a tracrRNA and a crRNA, which may be provided separately, or linked via covalent or non-covalent bonds/interactions. The guide RNA may also be a pegRNA of a Prime Editing system as further disclosed below. The at least one guide molecule may be provided in the form of one coherent molecule, or the sequence encoding the same, or in the form of two individual molecules, e.g., crRNA and tracr RNA, or the sequences encoding the same.

In certain embodiments, the genome editing system may be a base editor (BE) system.

In yet another embodiment, the genome editing system may be a Prime Editing system.

Any nucleic acid sequence comprised by, or encoding a genome modification or genome editing system disclosed herein, or a regeneration booster sequence, may be “codon optimized” for the codon usage of a plant target cell of interest. This means that the sequence is adapted to the preferred codon usage in the organism that it is to be expressed in, i.e, a “target cell of interest”, i.e., an IIM cell, which may have its origin in different target plants (wheat, maize, sunflower, sugar beet, for example) so that a different codon optimization may be preferable, even though the encoded effector on protein level may be the same. If a nucleic acid sequence is expressed in a heterologous system, codon optimization increases the translation efficiency significantly.

In certain embodiments according to the methods as disclosed herein, it may be preferable to achieve homology-directed repair (HDR)-mediated genome editing instead of In certain embodiments according to the various aspects and methods disclosed herein, wherein at least one genome editing system is introduced, the at least one genome editing system comprises at least one repair template (or donor), and wherein the at least one repair template comprises or encodes a double- and/or single-stranded nucleic acid sequence.

In a further embodiment of the genome editing system according to any of the embodiments described above, the system may thus additionally comprise at least one repair template, or a sequence encoding the same. A “repair template”, “repair nucleic acid molecule”, or “donor (template)” refers to a template exogenously provided to guide the cellular repair process so that the results of the repair are error-free and predictable. In the absence of a template sequence for assisting a targeted homologous recombination mechanism (HDR), the cell typically attempts to repair a genomic DNA break via the error-prone process of non-homologous end-joining (NHEJ).

In one embodiment, the at least one repair template may comprise or encode a double- and/or single-stranded sequence.

In another embodiment, the at least one repair template may comprise symmetric or asymmetric homology arms.

In a further embodiment, the at least one repair template may comprise at least one chemically modified base and/or backbone.

In one embodiment, a genome modification or editing system according to any of the embodiments described above, the at least one site-directed nuclease, nickase or an inactivated nuclease, or a sequence encoding the same, and/or optionally the at least one guide nucleic acid, or the sequence encoding the same, and/or optionally the at least one repair template, or the sequence encoding the same, are provided simultaneously, or one after another.

At least one repair template can be delivered with the at least one genome modification or editing system and/or the at least one regeneration booster simultaneously or subsequently with the proviso that it will be active, i.e., present and readily available at the site of a genomic target sequence in an IIM cell to be modified together with the at least one further tools of interest.

The repair template can be additionally introduced by bombardment at least one more time 1-8 hours after first bombardment, especially when genome editing components are delivered as sequences encoding the same to increase repair template availability for a targeted repair process.

In one embodiment, the at least one repair template may comprise symmetric or asymmetric homology arms.

In another embodiment, the at least one repair template may comprise at least one chemically modified base and/or backbone, including a phosphothioate modified backbone, or a fluorescent marker attached to a nucleic acid of the repair template and the like.

In certain embodiments, the at least one genome editing system, optionally the at least one regeneration booster, and optionally the at least one repair template, or the respective sequences encoding the same, are introduced transiently or stably, or as a combination thereof. Whereas the stable integration of at least one genome editing system, in particular the site-directed nuclease or variant thereof, but not necessarily including at least one guide RNA, may allow a stable expression of this effector, the methods as disclosed herein can be performed in a full transient way. This implies that the tools as such are not integrated into the genome of a cell to be modified, unless at least one repair template is used. This transient approach may be preferably for a highly controllable gene editing event.

In a preferred embodiment, the plants which may be subject to the methods and uses of the present invention are preferably monocot plants, including plants from the order of Poales, and most preferably the plants from the family of Poaceae, comprising the genus Agrostis, Aira, Aegilops, Alopecurus, Ammophila, Anthoxanthum, Arrhenatherum, Avena, Beckmannia, Brachypodium, Bromus, Calamagrostis, Coix, Cortaderia, Cymbopogon, Cynodon, Dactylis, Deyeuxia, Deschampsia, Elymus, Elytrigia, Eremopyrum, Eremochloa, Festuca, Glyceria, Helictotrichon, Hordeum, Holcus, Koeleria, Leymus, Lolium, Melica, Muhlenbergia, Poa, Paspalum, Polypogon, Oryza, Panicum, Phragmites, Pryza, Puccinellia, Saccharum, Secale, Sesleria, Setaria, Sorghum, Stipa, Stenotaphrum, Trisetum, Triticum, Zea, Zizania, or Zoysia.

In certain embodiments, plants with enlarged inflorescence meristem resulting from mutations (e.g., cauliflower, broccoli) may be used in the methods disclosed herein, i.e., plants of the genus Brassica, in particular Brassica oleracea var. botrytis L., and Brassica oleracea var. italica.

In another embodiment, the plants which may be subject to the methods and uses of the present invention are preferably dicot plants, including plants from the order of Helianthede or Betoidede, comprising the genus Helianthus or Beta.

In a further embodiment, the plant cell, tissue, organ, plant or seed disclosed in context of the present invention, originates from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant cell, tissue, organ, plant or seed originates from a species selected from the group consisting of Hordeum vulgare, Hordeum bulbusom, Sorghum bicolor, Saccharum officinarium, Zea spp., including Zea mays, Setaria italica, Oryza mimuita, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus and/or Spinacia oleracea.

In one aspect, there is provided plant cell, tissue, organ, plant or seed obtainable by or obtained by a method for plant genome modification as disclosed herein, wherein the plant cell, tissue, organ, plant or seed obtained may be a monocotyledonous (monocot) or a dicotyledonous (dicot) a plant cell, tissue, organ, plant or seed.

In certain embodiments, the inflorescence from a plant, for example, a Poaceae plant the at least one IIM cell to be modified according to the methods as disclosed herein originates from, may be a panicle, spike, or a raceme based on the morphological characteristics of the inflorescence. Each type has a spikelet, which may, however, have all kinds of shape. A spikelet is a pair of variously shaped bracts (also known as glumes, modified leaves) with enclosed floret(s). A floret is a small flower comprised of two bracts, which enclose the reproductive organs: stamens, comprised of anthers with supporting filaments, represent the male sex; the pistil, comprised of the stigma, style, and ovary represent the female sex.

The plant development is generally divided into vegetative, transition, and reproductive phases. Specifically, for embodiments referring to plants from the Poaceae family, the vegetative phase is characterized by the shoot meristem producing leaves and branches (tillers) and remaining at or near the soil surface. Vegetative phase includes 7 development stages: seed germination, first leaf emergency, first leaf, two leaves, three leaves, initial tillering, and tillering, sequentially. Transition phase is described by an elevation of the apical meristem and its transition to inflorescence meristem development. During the transition phase, leaf sheaths begin to elongate, raising the meristematic collar zone to a grazable height. Transition phase includes: shoot elongation, first node, second node, and third node. The reproductive phase defines the development stages of inflorescence meristem producing flowers and seeds, comprising of: flag leaf (flag leaf collar visible, pollen development starts), early boot, boot (which is defined as the time when the seed-head is enclosed within the sheath of the flag leaf), seed-head emergence, early anthesis, and anthesis.

In the case of Triticeae tribe species (e.g., including the important crops like wheat, barley, rye), the plants bear an inflorescence in the form of a spike, with a main axis of two ranks of lateral sessile distichous spikelets directly attaching to the rachis. The inflorescence development involves a series of morphological changes to the shoot apex-begins with spike initiation or spikelet formation, which occurs before the beginning of stem elongation. The transition of shoot apical meristem to inflorescence meristem triggers stem elongation. After the transition the inflorescence meristem develops ridges composed of bract primordia, followed by the development of spikelet meristems as axillary buds. The inflorescence meristem development is divided into four stages: 1) the double ridge/spikelet meristem (DR) stage when the spikelet meristem development is initiated and the first node is visible: 2) the floret meristem (FM) stage when the floret meristem development starts (it is marked by the emergency of the second node): 3) the anther meristem (AM) stage when anther meristems are formed, the flag leaf is emerging, and the third node begin to extend; and 4) the young floret stage when the styles have just emerged from the pistils (TS), and the flag leaf is elongating. At the young floret stage tetrads are formed in the elongating styles.

The development stages of maize (Zea mays) are also divided into vegetative, transition, and reproductive phases, morphologically.

In one aspect, there is thus provided a method of staging plants, i.e., a method of determining the developmental stage at which IIM cells according to the methods of the present disclosure, can be identified and obtained to be modified as disclosed herein.

The vegetative phase includes VE (the first leaf emergence) to V14 (the 14th leaf collar is visible) stages, transition phase occurs when tassel is emerging, while reproductive phase starts at R1 stage (silk is emerging) to R6 (kernel full maturity). Maize plant development includes the following stages in a sequential order:
VE: emergence of the first leaf
V1: the collar of the first leaf is visible
V2 to V14: the collar of the second leaf to the collar of 14th leaf is visible
VT: tassel emergence
R1: any silk is visible.
R2 to R6: kernels development starts to physiological maturity of the kernels.

From development point of view, the maize reproductive phase however starts quite early. The inflorescence meristem development initiates at V5 to V6 stages (plants with 5-6 visible leaf collars: see FIG. 1). At about the same time when the tassel is started, axillary meristem at leaf base node (behind the leaf sheath) transits to the ear primordium, where husk leave development is initiated, and followed by ear meristem at the tip of the ear shank. The transition of axillary meristem to ear primordium begins at the low leaf nodes of the stalk and continuing toward the top except for the upper six to eight nodes of the plant. By the development stage of V10, all inflorescent primordia initiation is initiated, and the potential number of rows (ear girth) is determined. The ear shoots located at the lower stalk nodes are first bigger than the ones at the upper stalk nodes because the lower ones were produced earlier. As development moves on, the upper one or two car shoots take on priority over all the lower ones and ultimately become the harvestable cars.

The skilled person is well aware of the fact that protocols, usually based on morphological characteristics are available for all relevant crop plants so that the teaching as provided herein can be transferred to other target plants for defining, isolating and/or providing immature inflorescence cells for transformation.

In certain embodiments, any immature inflorescences with underdeveloped floral bracts (e.g. glume, lemma, palea) may be preferred as immature inflorescence meristem cells to be transformed.

In one embodiment, for example in the case of Triticede tribe species as target plants, e.g., wheat, barley, rye, the immature inflorescences at the development stages of early double ridge/spikelet meristem (DR) to early young floret may be preferably subjected to the methods disclosed herein, preferably the immature inflorescences at late DR stage to late anther primordium (AM). In the case of maize, the immature tassels and cars are both applicable to the methods in the present invention. The immature tassels derived from the plants at the development stages of V5 to V10, and preferably from the plants at development stages of V6 to V8 may be particularly suitable for the methods disclosed herein. The immature cars from the plants at the development stages of V5 to V12, and preferably from the plants at development stages of V6 to V10, may also be applicable for the methods as uses disclosed herein.

The developmental stages of an inflorescence of a plant of interest may be determined by macroscopic, microscopic and/or molecular techniques, including visual inspection of plant morphology and growth, microscopy, e.g., using a stereo microscope, or by defining the expression of marker genes or metabolites characteristic of a special developmental stage. Such techniques are known to the skilled person for all relevant monocot and dicot crop plants and can be adapted based on the methods of staging plants to identify IIM cells as disclosed herein.

Plant cells for use according to the methods disclosed herein can be part of, or can be derived or isolated from any type of plant inflorescent meristems in intro, or in vivo. It is possible to use isolated plant cells as well as plant material, i.e, whole plants or parts of plants containing the plant cells. A part or parts of plants may be attached to or separated from a whole intact plant.

In certain embodiments, plant growth regulators like auxins or cytokinins in the tissue culture medium can be added to manipulated to induce callus formation and subsequently changed to induce embryos to form from the callus.

Somatic embryogenesis has been described to occur in two ways: directly or indirectly. Direct embryogenesis occurs when embryos are started directly from explant tissue creating an identical clone.

Indirect embryogenesis occurs when explants produced undifferentiated, or partially differentiated, cells (i.e, callus) which then is maintained or differentiated into plant tissues such as leaf, stem, or roots.

A variety of delivery techniques may be suitable according to the methods of the present invention for introducing the components of a genome modification or editing system and/or at least one booster gene and/or at least one transgene, or the respective sequences encoding the same, into a cell, in particular an IIM cell, the delivery methods being known to the skilled person, e.g, by choosing direct delivery techniques ranging from polyethylene glycol (PEG) treatment of protoplasts, procedures like electroporation, microinjection, silicon carbide fiber whisker technology, viral vector mediated approaches and particle bombardment. A common biological means, and a preferred cargo according to the present invention, is transformation with Agrobacterium spp, which has been used for decades for a variety of different plant materials. Viral vector mediated plant transformation represents a further strategy for introducing genetic material into a cell of interest.

A particularly preferred delivery technique may be the introduction by physical delivery methods, like (micro-) particle bombardment or microinjection. Particle bombardment includes biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Particle bombardment and microinjection have evolved as prominent techniques for introducing genetic material into a plant cell or tissue of interest. Helenius et al . . . “Gene delivery into intact plants using the Helios™ Gene Gun [gene gun]”. Plant Molecular Biology Reporter. 2000, 18 (3): 287-288 discloses a particle bombardment as physical method for introducing material into a plant cell.

The term “(micro-) particle bombardment” as used herein, also named “biolistic transfection” or “microparticle-mediated gene transfer” refers to a physical delivery method for transferring a coated microparticle or nanoparticle comprising boost genes, booster polypeptides, genome engineering components, and/or transgenes into a target cell or tissue. The micro- or nanoparticle functions as projectile and is fired on the target structure of interest under high pressure using a suitable device, often called gene-gun. The transformation via particle bombardment uses a microprojectile of metal covered with the construct of interest, which is then shot onto the target cells using an equipment known as “gene gun” (Sandford et al. 1987) at high velocity fast enough (−1500 km/h) to penetrate the cell wall of a target tissue.

but not harsh enough to cause cell death. For protoplasts, which have their cell wall entirely removed, the conditions are different logically. The precipitated construct on the at least one microprojectile is released into the cell after bombardment. The acceleration of microprojectiles is accomplished by a high voltage electrical discharge or compressed gas (helium). Concerning the metal particles used it is mandatory that they are non-toxic, non-reactive, and that they have a lower diameter than the target cell. The most commonly used are gold or tungsten. There is plenty of information publicly available from the manufacturers and providers of gene-guns and associated system concerning their general use.

In one embodiment using particle bombardment, the various components of a genome modification or editing system and/or at least one booster gene and/or at least one transgene, or the respective sequences encoding the same, are co-delivered via microcarriers comprising gold particles having a size in a range of 0.4-1.6 micron (pm), preferably 0.4-1.0 μm. In an exemplary process, 10-1,000 pg of gold particles, preferably 50-300 pg, are used per one bombardment.

The various components of a genome modification or editing system and/or at least one booster gene and/or at least one transgene, or the respective sequences encoding the same, can be delivered into target cells for example using a Bio-Rad PDS-1000/He particle gun or handheld Helios gene gun system. When a PDS-1000/He particle gun system used, the bombardment rupture pressures are from about 450 psi to 2200 psi, preferred from about 450 to 1800 psi, while the rupture pressures are from about 100-600 psi for a Helios gene gun system. More than one chemical or construct can be co-delivered with genome engineering components into target cells simultaneously. The above-described delivery methods for transformation and transfection can be applied to introduce the tools of the present invention simultaneously. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alia transfection with calcium phosphate, transfection using liposomes, e.g., cationic liposomes, or transfection with cationic polymers, including DEAD-dextran or polyethylenimine, or combinations thereof. The above delivery techniques, alone or in combination, can be used for in vivo (including in planta) or in vitro approaches. Particle bombardment may have the advantage that this form of physical introduction can be precisely timed so that the material inserted can reach a target compartment together with other effectors in a concerted manner for maximum activity. IIM cells were shown to be particularly susceptible to particle bombardment and tolerate this kind of introduction well.

In one embodiment, more than one different transformation/transfection technique as disclosed above is combined, preferably, wherein at least one of the components of a genome modification or editing system and/or at least one booster gene and/or at least one transgene, or the respective sequences encoding the same, is introduced via particle bombardment.

In certain embodiments, the methods for plant genome modification as disclosed herein may comprise a preceding step of preparing plant cells as part of preferably immature inflorescence meristem (IIM) for providing at least one immature inflorescence meristem cell.

In certain embodiments of the methods disclosed herein, the regeneration of the at least one modified cell may be a direct meristem regeneration comprising the steps of: shoot meristem induction for about 1 to 4 weeks, preferably 10-25 days, shoot meristem propagation for about 1 to 4 weeks, preferably 10-25 days, shoot outgrowth for about 1 to 4 weeks, preferably 10-20 days, and root outgrowth for about 1 to 4 weeks, preferably 3-20 days.

In other embodiments of the methods disclosed herein, the regeneration of the at least one modified cell may be an indirect meristem regeneration comprising the steps of: inducing embryogenic callus formation for about 1 to 6 weeks, preferably 2-4 weeks, most preferably 3 weeks, shoot meristem development and outgrowth for about 1 to 6 weeks, preferably 2-4 weeks, most preferably 10-25 days, and root outgrowth for about 1 to 4 weeks, preferably 3-14 days; and optionally: screening for genetic modification events in the regenerated TO plants; and further optionally: growing the modified TO plants for T1 seed production and optionally screening T1 progeny for desirable genetic modification events.

In a further aspect, there is provided a generally applicable expression construct assembly, which may be used according to the methods disclosed herein, wherein the expression construct assembly comprises (i) at least one vector encoding at least one site-directed nuclease, nickase or an inactivated nuclease of a genome editing system, preferably wherein the genome editing system is as defined above, and (ii) optionally: at least one vector encoding at least one regeneration booster, preferably wherein the regeneration booster is as defined above, and (iii) optionally, when the at least one site-directed nuclease, nickase or an inactivated nuclease of a genome editing system is a nucleic acid guided nuclease: at least one vector encoding at least one guide molecule guiding the at least one nucleic acid guided nuclease, nickase or an inactivated nuclease to the at least one genomic target site of interest; and (iv) optionally: at least one vector encoding at least one repair template: wherein (i), (ii), (iii), and/or (iv) are encoded on the same, or on different vectors.

In one embodiment, the expression construct assembly may further comprise a vector encoding at least one marker, preferably wherein the marker is introduced in a transient manner, see, for example, SEQ ID NO: 48.

In one embodiment, the expression construct assembly comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

Notably different components of a genome modification or editing system and/or a regeneration booster sequence and/or a guide molecule and/or a repair template present on the same vector of an expression vector assembly may be comprise or encode more than one regulatory sequence individually controlling transcription and/or translation.

In one embodiment of the expression construct assembly described above, the construct comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

In another embodiment of the expression construct assembly described above, the regulatory sequence comprises or encodes at least one promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof. In a further embodiment of the expression construct assembly described above, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

In one embodiment of the expression construct assembly according to any of the embodiments described above, the construct comprises or encodes a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and a AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In addition, the expression construct assembly may comprise at least one terminator, which mediates transcriptional termination at the end of the expression construct or the components thereof and release of the transcript from the transcriptional complex.

In one embodiment of the expression construct assembly according to any of the embodiments described above, the regulatory sequence may comprise or encode at least one terminator selected from the group consisting of nosT, a double 35S terminator, a ZmEf1 terminator, an AtSac66 terminator, an octopine synthase (ocs) terminator, or a pAG7 terminator, or a combination thereof. A variety of further suitable promoter and/or terminator sequences for use in expression constructs for different plant cells are well known to the skilled person in the relevant field.

The methods as disclosed herein, in particular for transient particle bombardment and direct meristem regeneration of IIM cells, are highly effective and efficient and able to achieve single-cell origin regeneration and homogenous genome editing without a conventional selection (e.g., using an antibiotic or herbicide resistant gene).

Exemplary elements of an expression vector assembly of the present invention, which may be individually combined, may comprise (i) a suitable vector backbone, for example, according to SEQ ID NO: 34, wherein a variety of suitable vectors are available in plant biotechnology: (ii) an expression cassette, i.e., a cassette encoding a sequence of an effector, for example, at least one regeneration booster as disclosed herein, for example, according to any one of SEQ ID NOs: 23 to 33: (iii) an expression construct, i.e., a construct including an expression cassette and at least one further vector element, for example, as represented in any one of SEQ ID NOs: 35 to 43, or an empty vector, for example, according to SEQ ID NO: 34: (iv) a vector or expression construct comprising or encoding at least one site-directed nuclease, for example, as represented in any one of SEQ ID NOs: 46 and 50: (v) a suitable vector encoding a guide molecule, in case a nucleic acid-guided site directed nuclease is used, specific for a genomic target sequence of interest, for example, a sequence according to any one of SEQ ID NOs: 47, 49, and 50, wherein the respective guide molecule is compatible with the cognate nucleic acid-guided site directed nuclease, or variant thereof, wherein the respective guide molecule comprised or encoded by according to any one of SEQ ID NOs: 47, 49, and 50 can be easily replaced by another guide molecule targeting a different genomic target site of interest: (vi) a vector encoding at least one repair template sequence of interest; and/or (vii) a vector or expression construct comprising or encoding at least one expressible marker gene, preferably a marker gene, which can be easily detected macroscopically, or microscopically, like a fluorescent marker gene as encoded by, for example, SEQ ID NO: 48. A variety of suitable fluorescent marker proteins and fluorophores applicable over the whole spectrum, i.e., for all fluorescent channels of interest, for use in plant biotechnology for visualization of metabolites in different compartments are available to the skilled person, which may be used according to the present invention. Examples are GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum), an orange, a red or far-red fluorescent protein (e.g., tdTomato (tdT), or DsRed), and any of a variety of fluorescent and coloured proteins may be used depending on the target tissue or cell, or a compartment thereof, to be excited and/or visualized at a desired wavelength.

All elements of the expression vector assembly can be individually combined. Further, the elements can be expressed in a stable or transient manner, wherein a transient introduction may be preferably. In certain embodiments, individual elements may not be provided as part of a yet to be expressed (transcribed and/or translated) expression vector, but they may be directly transfected in the active state, simultaneously or subsequently, and can form the expression vector assembly within one and the same IIM cell of interest to be modified. For example, it may be reasonable to first transform part of the expression vector assembly encoding a site-directed nuclease, which takes some time until the construct is expressed, wherein the cognate guide molecule is then transfected in its active RNA stage and/or at least one repair template is then transfected in its active DNA stage in a separate and subsequent introduction step to be rapidly available. The at least one regeneration booster sequence and/or the at least one genome modification or editing system and/or the at least one marker may also be transformed as part of one vector, as part of different vectors, simultaneously, or subsequently. The use of too many individual introduction steps should be avoided, and several components can be combined in one vector of the expression vector assembly, to reduce cellular stress during transformation/transfection. In certain embodiments, the individual provision of elements of the at least one regeneration booster sequence and/or the at least one genome modification or editing system and/or the at least one marker and/or the at least one guide molecule and/or the at least one repair template on several vectors and in several introduction steps may be preferable in case of complex modifications relying on all elements so that all elements are functionally expressed and/or present in a cell to be active in a concerted manner.

The present invention is further illustrated by the following non-limiting Examples.

### EXAMPLES

### Example 1: Plant Immature Inflorescence Preparation for Different Target Plants

### A: Maize Plant Cultivation

Depending on seed germination rate, 1-2 seeds per well are planted into a deep 50-well plug tray (FIG. 1A) placed further within a tray without holes (FIG. 1B). After germination only one seedling per well is kept. The soil used is MetroMix360/Turface 3:1 blend. The seeds are germinated and growing in a growth chamber at 28° C., for day and 22° C., for night, light intensity of 400-600 μmol m⁻² s⁻¹ and 14-16 hours day length, 50% humidity. Maize plants are fertilized at every watering using Jack's 15-5-15 Ca—Mg diluted to 150 ppm nitrogen. Plants are watered as needed.

It is a huge advantage that this cultivation method is not associated with pollen contamination issues, therefore, allowing that multiple genotypes can be grown in in the same tray, as shown in FIG. 2. The outer dimensions of the deep 50-well tray as shown in FIG. 2 were chosen as follows: 21¼″×11¼″×2¼, with cell dimensions: 1¾″×1¾″. Obviously, these dimensions may vary depending on the target plant (and thus the morphological characteristics thereof) of interest.

### B: Maize Immature Tassel Isolation

Maize immature tassels from the plants at late V6 to late V7 stages (FIG. 2) are used. It most likely takes 25-32 days to reach these stages for different genotypes after seed planting when cultured at the growth conditions described above. The developmental stages of immature tassels are determined using, for example, a Zeiss stereo microscope. Maize immature tassel isolation usually comprises the steps of
1. Harvesting stalk segment with immature tassel from maize plants by retrieving the stalk segment between the base (near to soil) and the youngest leaf collar and removing all the leaf blades;
2. (Optional) Rinsing the stalk segments with tap water, and dry with paper towel;
3. Surface spraying the stalk segments with 70% ethanol, and manually removing the first leaf sheath from stalk in a laminar hood;
4. Repeating step 3, and carefully remove each leaf sheaths from stalk, one by one from the bottom to top, until the last 3rd leaf sheath;
5. Trimming the stalk segments and spraying them with 70% ethanol (the last ethanol spray);
6. Transferring the segments onto a clear petri dish in the hood;
7. Under a dissection microscope: carefully removing all remaining leaves so that the immature tassels are ready for transformation (FIG. 3A).

The isolated maize immature tassel comprising of primary and branch inflorescence meristems, and which further comprising of spikelet meristem. The floral bract primordia are underdeveloped and the inflorescence meristem is open (FIG. 3A).

### C: Maize Immature Ear Isolation

Maize immature ears used for the methods in the present invention are derived from the plants at the development stages of V8 to late V10. It most likely takes 5-6 weeks from seed planting to the immature ear harvesting. Ears are located at each of stalk nodes, enclosed by the leaf sheath, and normally surrounded by husk leaves. The developmental stages of immature ears are determined using, for example, a Zeiss stereo microscope. Maize immature ear isolation comprises the steps of
1. Harvesting the stalk with immature ears from corn plants by retrieving the stalk segment between the base (near to soil) and the youngest leaf collar and removing all the leaf blades;
2. (Optional) Rinsing the stem segments with tap water, and drying with paper towel;
3. Surface spraying the stalk segments with 70% ethanol, and manually removing the first leaf sheath from stalk, and isolating the first immature ear shoot carefully at the first stalk node in a laminar hood;
4. Repeating step 3, isolating all immature ear shoots at each of the stalk nodes, one by one from the bottom to top, in the hood;
5. Spraying the ear shoots with 70% ethanol (the last ethanol spray), and transferring the shoots into a clear petri dish;
6. Under a dissection microscope: carefully removing all the husks from each of ear shoots, and the immature ear meristems are ready for transformation.

Isolating maize immature ears comprising of ear spikelet in pairs of rows. The floral bract primordia are underdeveloped and the inflorescence meristem is open (FIG. 3B).

### D: Rye Plant Cultivation

KWS bono rye were grown in a growth chamber. Two KWS bono rye seeds are planted into a 1801 deep inserts plug pot (placed into a 18-count holding tray without holes). After germination, only one seedling per pot is kept.

The soil used was Berger 35% Bark. The seeds are germinated and growing in a growth chamber at constant 20° to 21° C., with light intensity of 400-600 μmol m² s⁻¹ and 14 hours day length, 50% humidity. The rye plants were fertilized three time a week with Jack's 15-16-17 peat lite at an E.C, of 1.0+the E.C, of the water. The plants were checked twice a day for watering needs and are watered from top as needed.

After the plants have germinated and produced one to two tillers, the plants were moved to a vernalization chamber at a temperature of 0° to 5° C., for 40 days. Once they are moved back to the normally growth condition they will begin their reproductive development.

### E: Rye Immature Inflorescence Isolation

The developmental stages of rye immature inflorescences were determined using a Zeiss stereo microscope. When the first node of stem was visible the inflorescences of a rye plant are in DR (double ridge/spikelet meristem stage) stage. About 1 week later, the second node is emerging, floret meristem development begins, and the plant is in FM (floret meristem) stage. After 5-7 more days, the third stem internode begins to elongate, and anther primordia are visible, and the plant is in AM (anther primordium/meristem) stage and is ready to enter the booting stage.

Rye immature inflorescences at the development stages of late double ridge (DR) to late anther meristem (AM) are used for the methods in the present invention. At these stages the rye shoots are elongated with 1-3 visible nodes.

The rye immature inflorescence isolation comprises the steps of:
1. Harvesting the rye shoots at the right development stages from the base of the stalks (near to soil) and removing all the leaf blades;
2. (Optional) Rinsing the stem segments with tap water, and drying with paper towel;
3. Surface spraying the shoots with 70% ethanol, and manually removing the first leaf sheath from stalk in a laminar hood;
4. Repeating step 3, and carefully removing each leaf sheaths from stalk, one by one from the bottom to top, until the flag leaf sheath;
5. Trimming the stalk segments and spray with 70% ethanol (the last ethanol spray);
6. Transferring the segments onto a clear petri dish in the hood;
7. Under a dissection microscope: carefully removing the flag leaf sheath, and all of immature bracts, and the immature inflorescence is ready for transformation (FIG. 3C).

### Example 2: Transient Biolistic Transformation of Immature Tassels from Maize Elites

Maize immature tassel preparation was performed as detailed above (Example 1).

### Microparticle Bombardment

The freshly isolated immature tassels from different inbred elites were placed onto an osmatic medium plate (e.g. IM_OS medium) for 4 hours. Particle bombardment was conducted using a Bio-Rad PDS-1000/He particle gun. The bombardment conditions were: 30 mm/Hg vacuum, 1,350 psi helium pressure. Per bombardment, 200 ng of plasmid DNA pGEP837 (FIG. 4) was coated onto 100 μg of 0.6 μm gold particles using calcium-spermidine method. Four bombardments per sample plate were performed. The bombarded immature tassels were kept on the osmotic medium plate for another 20 hours after the bombardment. A green fluorescence reporter encoding gene (FIG. 4) was used for monitoring biolistic transformation and the gene expression. Efficient transient expression of the reporter gene was observed in all tested inbred elites 20 hours after the bombardment (FIG. 5).

### Example 3: Efficient Genome Editing by Transient Biolistic Transformation and Direct Meristem Regeneration from Maize A188 Immature Tassel

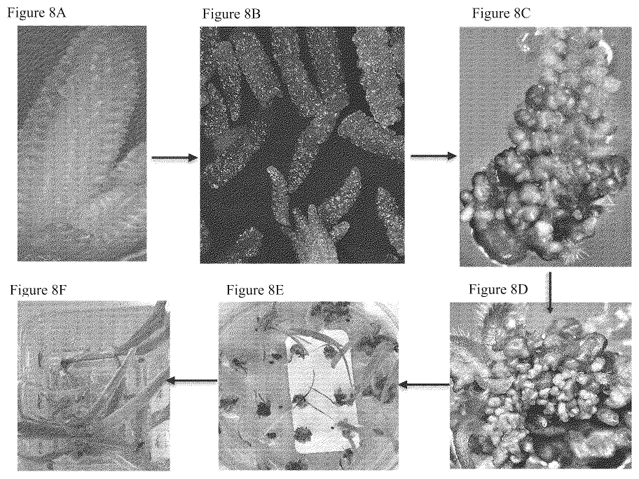

A freshly isolated immature tassel from 28-day-old A188 seedling is shown in FIG. 8A, which was prepared as described in Example 1 and bombarded as detailed in Example 2.

Construct pGEP837 contains the expression cassette of CRISPR nuclease MAD7 (FIG. 4), while pGEP842 harbors the expression cassette of CRISPR sgRNA m7GEP1, which targets to maize endogenous HMG13 gene (FIG. 6). Construct pABM-BdEF1_ZmPLT5 encloses a maize regeneration boost gene PLT5 expression cassette (FIG. 7).

200 ng of plasmid DNA pGEP837, 300 ng of plasmid DNA pGEP842, and 100 ng of pABM-BdEF1_ZmPLT5, were co-coated onto 100 μg of 0.4 μm gold particles using calcium-spermidine method, and the three constructs were co-delivered into the cells of A188 immature tassel (FIG. 8A) by particle bombardment at 1,350 psi rupture pressure, 4 bombardment shots per sample plate. The bombarded A188 immature tassel was kept on the osmotic IM_OS plate for another 20 hours after the bombardment. Efficient transient expression of the reporter gene could be demonstrated as shown in FIG. 8B.

### A: Direct Meristem Regeneration of Maize Immature Tassels

20 hours after the bombardment the A188 immature tassel was subject to direct meristem regeneration, which comprising the steps of:

Step I: cutting the bombarded immature tassel branches into a segment of 3-5 mm in length with a sharp blade, and placing them onto an IMSMK5 medium petri dish plate (25×100 mm) at a density of 12 pieces per plate. Sealing the plate with surgical tape, and culturing at 27° C., dark for 2 days, and then transferring the plates and culture at 25° C., weak light (10˜50 μmol m⁻² s⁻¹, gradually increase light intensity), 16/8 h light/dark cycle for a total of 14 days (FIG. 8C).

Step II: removing the developing bracts or leaves, and separating the meristem buds from the step I into small pieces in 2-5 mm diameter, and transferring the meristem buds onto a fresh IMSMK5 medium. Sealing the plate with surgical tape, and culturing at 25° C., light (˜100 μmol m⁻² s⁻¹), 16/8 h light/dark cycle, for 2 weeks (FIG. 8D).

Step III: separating the developing shoot buds from the step II, and transferring the shoot buds onto a Shooting medium petro dish (25×100 mm). Sealing the plate with surgical tape and culture at 25° C., light (˜100 μmol m⁻² s⁻¹) for 2 weeks (FIG. 8E).

Step IV: removing the developing shoots from step III onto a Rooting medium in phytotray, and culturing at 25° C., light (˜100 μmol m⁻² s⁻¹) for 1 week (FIG. 8F).

After 1 week at regeneration step IV, the regenerated plantlets (FIG. 8F) are ready for leaf sampling for molecular analysis or transfer to soil for T₀ plant growth and T1 seed production.

The work flow of the direct meristem regeneration of immature tassels is summarized in summarized in Table 1:

TABLE 1
Work flow for SDN-1 generation from maize
immature tassel via direct meristem regeneration.

| | | Culture | |
|---|---|---|---|
| | Media | conditions | Duration |
| Step I: SM induction | IMSMK5 | 27° C., dark | 2 weeks |
| Step II: SM development | IMSMK5 | 25° C., light | 2 weeks |
| Step III: Shooting | Shooting medium | 25° C., light | 2 weeks |
| Step III: rooting | Rooting medium | 25° C., light | 1 weeks |

In sum, seventy-two (72) T₀ plantlets were regenerated from the 28-day-old A188 immature tassel (FIG. 8A) after the biolistic transformation of the CRISPR constructs. The results are summarized in Table 2:

TABLE 2
SDN-1 efficiency in the regenerated T0 plantlets from a
28-day-old A188 immature tassel by direct meristem regeneration.

| No. plantlets | No. Bi-allelic | No. mono-allelic | Total | % |
|---|---|---|---|---|
| regenerated | SDN-1 | SDN-1 | SDN-1 | SDN-1 |
| 72 | 12 | 1 | 13 | 18% |

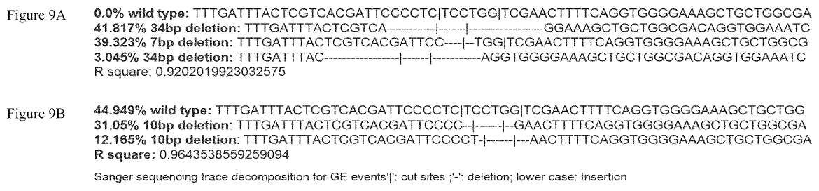

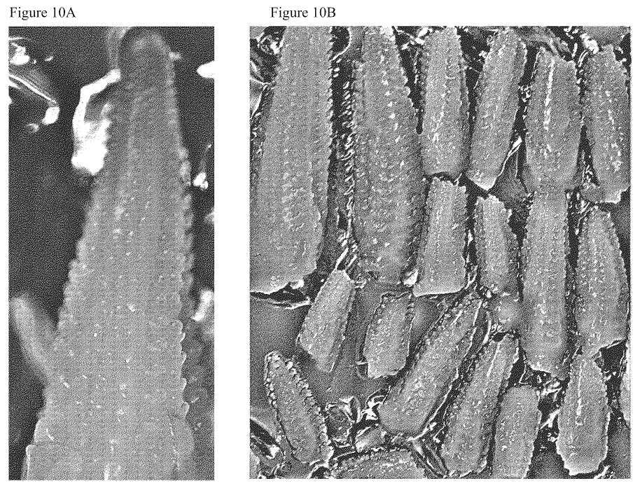

Molecular screening for the SDN-1 editing using Sanger sequencing coupled with sequence trace decomposition analysis identified 12 bi-allelic and 1 mono-allelic SDN-1 events from the 72 T₀ plants, which gives an 18% SDN-1 efficiency (Table 2). A representative sequencing result for a bi-allelic SDN-1 (FIG. 9A) or a mono-allelic SDN-1 event in the T₀ plants is shown in FIG. 9B. These results demonstrate that the methods in the present invention by transient particle bombardment and direct meristem regeneration of the cells as part of preferably immature tassel are highly effective and efficient; that, most importantly, the methods are able to achieve single-cell origin regeneration and homogenous genome editing without a conventional selection (e.g. using an antibiotic or herbicide resistant gene) in maize A188.

### Example 4: The Genome Editing Events from Transient Biolistic Transformation and Direct Meristem Regeneration of Maize A188 Immature Tassel are Fully Inheritable

Four edited T₀ plantlets from Example 3 were transferred into soil and the T1 seeds were produced by selfing or backcrossing to WT A188. Mature T1 seeds were soaked in water for about 24 hours, and the T1 embryos were isolated from the T1 seeds for DNA extraction individually. The SDN-1 segregations in the T1 progeny were analyzed by Taqman real time PCR. The results were shown in Table 3 below. The SDN-1 segregation ratios in the T1 progeny from all the tested lines perfectly match to the expectation from the Mendel's law of segregation for a genetic unit, and thus the SDN-1 modification events generated by using the methods from the present invention are fully inheritable (Table 3). These results also are in support of that the methods are able to achieve single-cell origin regeneration and homogenous genome editing without a conventional selection in maize A188.

TABLE 3
SDN-1 segregation ratios in the T1 progeny from 4
T0 lines derived from a 28-day-old A188
immature tassel by direct meristem regeneration.

| | | | SDN-1 ratio in T₁ |
|---|---|---|---|
| To edited event | SDN-1 in T₀ | Crossing | (WT:mono-:Bi-) |
| xxx-T-0012 | Bi-allelic | selfing | 0:0:9 |
| xxx-T-0013 | Bi-allelic | backcrossing to WT | 0:5:0 |
| xxx-T-0014 | Mono-allelic | sefling | 1:3:2 |
| xxx-T-0017 | Bi-allelic | selfing | 0:0:5 |

### Example 5: Genome Editing SDN-1 Via Immature Ear Bombardment and Direct Meristem Regeneration from Maize Elites

Three maize seedlings of elite 4V-40171 at V8 stage, 39 days after planting, were harvested for immature ear isolation. For the information about plant immature inflorescence, see Example 1.

11 immature ears were isolated from the three seedlings, which are comprising of ear spikelet in pairs of rows. The floral bract primordia are underdeveloped and the inflorescence meristem is open (FIG. 10).

The immature ears were osmotically treated in IM_OSM medium for 4 hours before the bombardment (FIG. 10B). For each bombardment, 200 ng of plasmid DNA pGEP837, 300 ng of plasmid DNA pGEP842, and 100 ng of pABM-BdEF1_ZmPLT5, were co-coated onto 100 μg of 0.4 μm gold particles using calcium-spermidine method, and the three constructs were co-delivered into the cells of maize 4V-40171 immature ears by the particle bombardment at 1,350 psi rupture pressure, 4 bombardment shots per sample plate. After 20 hours of post-bombardment osmotic treatment on the IM_OSM plate, the bombarded immature ears were subjected to the direct meristem regeneration procedure. For the detailed information on the particle bombardment and direct meristem regeneration of the immature ears, see Example 3.

154 plantlets were regenerated from the 11 bombarded immature ears of maize elite 4V-40171, which demonstrate high regeneration capability of the immature ear from the maize elite using the methods from the present invention. After 1 week, development in the Rooting medium in phytotray, a 5-10 mm leaf tip from each of the leaves of the 154 T₀ plantlets are collected for DNA extraction. Genome editing SDN-1 in the regenerated T₀ plants were screened using TaqMan Digital Droplet PCR. Five T₀ plants with significant SDN-1 modifications were identified. The typical Taqman ddPCR results are shown in Table 4 below. These results indicate the possibility of direct regeneration and genome editing in maize inbred elites by transient particle bombardment and direct meristem regeneration of the cells as part of preferably immature ears.

TABLE 4
Positive SDN-1 events identified from the 154 regenerated T0
plantlets derived from the 4V-40171 immature ears via ddPCR analysis.

| | WT | Target | Accepte | WT | Positive | SDN- |
|---|---|---|---|---|---|---|
| Sample ID | Conc | Conc | Droplets | Droplets | Droplets | 1% |
| xxx004-T-110 | 606 | 0.276 | 14253 | 5737 | 2 | 0 |
| xxx004-T-111 | 773 | 0.301 | 15064 | 7254 | 2 | 0 |
| xxx004-T-112 | 1043 | 0 | 14800 | 8699 | 0 | 0 |
| xxx004-T-113 | 360 | 184 | 17857 | 4702 | 1901 | 33.8 |
| xxx004-T-114 | 497 | 15.7 | 19765 | 6811 | 172 | 3.07 |
| xxx004-T-121 | 405 | 32.5 | 18342 | 5343 | 354 | 7.42 |
| xxx004-T-122 | 744 | 32.6 | 16207 | 7597 | 235 | 4.19 |
| xxx004-T-138 | 4.87 | 218 | 17431 | 72 | 2940 | 97.8 |

Compared to the results obtained from the maize A188 in Example 3, the highly chimeric SDN-1 modifications in the elite TO plants also suggest that the mitotic activities in the elite cells may be significantly lower than those from A188 cells (A188 is a highly regenerative maize genotype).

### Example 6: Efficient Genome Editing by Transient Biolistic Transformation and Indirect Callus Regeneration with Regeneration Boosters from Maize A188 Immature Tassel

For the information about the immature tassel preparation and the particle bombardment, see Examples 1, 2, and 3.

Two A188 immature tassels at V7 stage were harvested, and osmotically treated in two N6_OSM plates (one tassel per plate) for 4 hours before the bombardment. Construct pABM-BdEF1_KWS_RBP4 and pABM-BdEF1_KWS_RBP5 harbor the maize regeneration boost gene KWS_RBP4 (FIG. 11) and KWS_RBP5 expression cassette (FIG. 12), respectively. For each bombardment 200 ng of plasmid DNA pGEP837 and 300 ng of plasmid DNA pGEP842, and 100 ng each of pABM-BdEF1_ZmPLT5 plus either pABM-BdEF1_KWS_RBP4 or pABM-BdEF1_KWS_RBP5, were co-coated onto 100 μg of 0.4 μm gold particles using calcium-spermidine method, and the four constructs were co-delivered into the A188 immature tassel cells by the particle bombardment at 1,350 psi rupture pressure, 4 bombardment shots per sample plate. After 20 hours of post-bombardment osmotic treatment on the N6_OSM plate, the bombarded immature ears were subjected to the indirect callus regeneration procedure, which comprises the steps of:

Step I—embryogenic callus induction: cutting the bombarded immature tassel into a segment of 3-6 mm in length, with a sharp blade, and placing it onto a callus induction medium N6_5Ag in petro dish plate (25×100 mm). Sealing the plate with surgical tape and culture at 27° C., dark, for 3 weeks.

Step II—shoot development and outgrowth: separating of developing embryogenic calluses from the step I into small pieces 2-5 mm in diameter, and transferring the calluses onto a Shooting medium petro dish plate (25×100 mm). Seal the plate with surgical tape and culture at 25° C., light (20-100 μmol m² s⁻¹, gradually increase the light intensity) for 18 days.

Step III—root outgrowth: removing the developing shoots from step III onto a Rooting medium phytotray, and culture at 25° C., light (100 μmol m² s⁻¹) for ˜7 days.

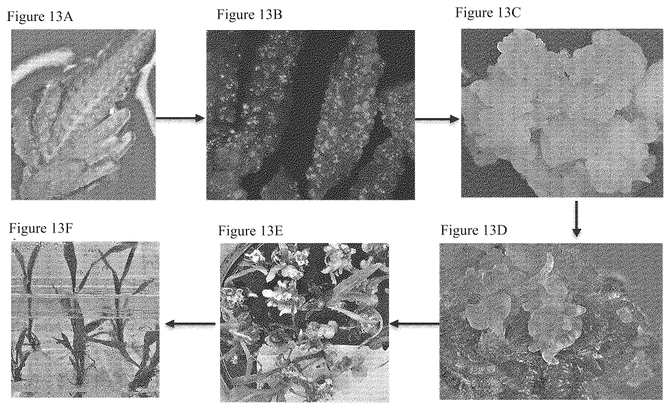

The work flow of the indirect callus regeneration is demonstrated in FIG. 13 and summarized in Table 5.

TABLE 5
Workflow for SDN-1 generation from maize immature
tassel via indirect callus regeneration.

| | | Culture | |
|---|---|---|---|
| | Media | conditions | Duration |
| Step I: callus induction | N6_5Ag/IMCIM2* | 27° C., dark | 3 weeks |
| Step II: shooting | Shooting medium | 25° C., light | 2-3 weeks |
| Step III: Rooting | Rooting medium | 25° C., light | 1 weeks |

After one week at regeneration step III, the regenerated plantlets are ready for leaf sampling for molecular analysis or transfer to soil for T1 seed production. For the information on the sampling molecular analysis, see Examples 3, 4, and 5.

Next, fifty-eight (58) plantlets were regenerated from the immature tassel co-bombarded with the boost ZmPLT5 and KWS_RBP4 constructs. 42, out of the 58 regenerated plants were screened for the SDN-1 editing by Sanger sequencing and trace decomposition analysis, and a total of 21 SDN-1 events were identified from the 42 screened T₀ plants, which gave a 50% SDN-1 efficiency from this A188 immature tassel.

From the A188 immature tassel that co-bombarded with the boost ZmPLT5 and KWS_RBP5, 80 plantlets were regenerated. 34, out of the 80 T₀ plants were screened for the SDN-1 by the Sanger sequencing and trace decomposition analysis. The results showed that 30 plants from the 34 tested T₀ plants had a bi-allelic SDN-1 editing in the target site, which gave an 88% SDN-1 efficiency. The results are summarized in Table 6, and the Sanger sequencing and trace decomposition analysis results from the 34 tested plants were displayed in Table 7, where the four T₀ plants with wild type sequence at the target side were highlighted in bold.

TABLE 6
SDN-1 efficiency in the regenerated T0 plantlets from a 28-day-old A188
immature tassel via the indirect callus regeneration with boosters.

| | | | | | Total | |
|---|---|---|---|---|---|---|
| | | | No. | No. | No. | |
| | | | events | events | events | |
| | No. | No. | with | with | with | % |
| | Regen | events | ~100% | ~50% | ≥50% | SDN-1 |
| Boosters | events | analyzed | SDN-1 | SDN-1 | SDN-1 | per event |
| ZmPLT5/RBP4 | 58 | 42 | 7 | 14 | 21 | 50% (21/42) |
| ZmPLT5/RBP5 | 80 | 34 | 30 | 0 | 30 | 88% (30/34) |

TABLE 7
Sanger sequencing trace decomposition analysis of genome editing events in 34 regenerated
T0 plantlets from a 28-day-old A188 immature tassel by indirect callus regeneration with booster
ZmPLT5 and KWS_RBP5.

| | | | | | | | | | |
|---|---|---|---|---|---|---|---|---|---|
| xxxx-T-001 | 23.969 | 9 bp | 13.81 | 9 bp | 11.44 | 10 bp | 0 | wild | square: |
| | | deletion | | deletion | | deletion | | type | 0.7586479701707 |
| xxxx-T-002 | 43.433 | 13 bp | 24.378 | 9 bp | 20.162 | 9 bp | 0 | wild | square: |
| | | deletion | | deletion | | deletion | | type | 0.9230283683580 |
| xxxx-T-003 | 31.135 | 6 bp | 19.999 | 16 bp | 19.607 | 16 bp | 0 | wild | square: |
| | | deletion | | deletion | | deletion | | type | 0.9161726174929 |
| xxxx-T-004 | 49.582 | 22 bp | 34.499 | 7 bp | 2.488 | 7 bp | 0.012 | wild | square: |
| | | deletion | | deletion | | deletion | | type | 0.9183147245080 |
| xxxx-T-005 | 39.686 | 13 bp | 23.767 | 9 bp | 23.598 | 9 bp | 0 | wild | square: |
| | | deletion | | deletion | | deletion | | type | 0.9194836171038 |
| xxxx-T-006 | 30.813 | 13 bp | 19.897 | 9 bp | 19.475 | 9 bp | 0 | wild | square: |
| | | deletion | | deletion | | deletion | | type | 0.9205654657684 |
| xxxx-T-007 | 43.88 | 13 bp | 21.511 | 9 bp | 20.344 | 9 bp | 0 | wild | square: |
| | | deletion | | deletion | | deletion | | type | 0.9081628415044 |
| xxxx-T-008 | 41.686 | 8 bp | 33.357 | 5 bp | 6.722 | 5 bp | 0 | wild | square: |
| | | deletion | | deletion | | deletion | | type | 0.9080586718376 |
| xxxx-T-009 | 29.83 | 6 bp | 20.519 | 16 bp | 18.98 | 16 bp | 0 | wild | square: |
| | | deletion | | deletion | | deletion | | type | 0.9119625939519 |
| xxxx-T-010 | 99.897 | wild | 0.031 | 34 bp | 0.026 | 34 bp | ### | | square: |
| | | type | | deletion | | deletion | | | 0.9999741786167 |
| xxxx-T-011 | 37.373 | 16 bp | 32.891 | 6 bp | 6.645 | 6 bp | 0 | wild | square: |
| | | deletion | | deletion | | deletion | | type | 0.9138174247368 |
| xxxx-T-012 | 31.977 | 6 bp | 24.087 | 16 bp | 22.677 | 16 bp | 0 | wild | square: |
| | | deletion | | deletion | | deletion | | type | 0.9068330392104 |
| xxxx-T-013 | 35.839 | 16 bp | 30.809 | 6 bp | 11.342 | 6 bp | 0 | wild | square: |
| | | deletion | | deletion | | deletion | | type | 0.9074834444915 |
| xxxx-T-014 | 47.815 | 13 bp | 20.629 | 9 bp | 18.797 | 9 bp | 0 | wild | square: |
| | | deletion | | deletion | | deletion | | type | 0.9178566734222 |
| xxxx-T-015 | 24.952 | 10 bp | 23.092 | 9 bp | 21.792 | 9 bp | 0 | wild | square: |
| | | deletion | | deletion | | deletion | | type | 0.8641021002026 |
| xxxx-T-016 | 32.205 | 9 bp | 26.368 | 10 bp | 14.677 | 9 bp | 0 | wild | square: |
| | | deletion | | deletion | | deletion | | type | 0.8690421762185 |
| xxxx-T-017 | 27.035 | 9 bp | 25.743 | 10 bp | 21.271 | 9 bp | 0 | wild | square: |
| | | deletion | | deletion | | deletion | | type | 0.8754330458572 |
| xxxx-T-018 | 44.017 | 13 bp | 23.208 | 9 bp | 19.782 | 9 bp | 0 | wild | square: |
| | | deletion | | deletion | | deletion | | type | 0.9199708107525 |
| xxxx-T-019 | 42.385 | 13 bp | 25.056 | 9 bp | 18.738 | 9 bp | 0 | wild | square: |
| | | deletion | | deletion | | deletion | | type | 0.9152609509911 |
| xxxx-T-020 | 29.088 | 6 bp | 23.886 | 16 bp | 23.577 | 16 bp | 0 | wild | square: |
| | | deletion | | deletion | | deletion | | type | 0.9015976483997 |
| xxxx-T-021 | 33.19 | 6 bp | 20.959 | 16 bp | 20.191 | 16 bp | 0 | wild | square: |
| | | deletion | | deletion | | deletion | | type | 0.9104306231335 |
| xxxx-T-022 | 42.793 | 13 bp | 23.076 | 9 bp | 21.328 | 9 bp | 0 | wild | square: |
| | | deletion | | deletion | | deletion | | type | 0.9239473757704 |
| xxxx-T-023 | 99.989 | wild | 0.007 | 9 bp | 0 | 1 bp | ### | | square: |
| | | type | | Insertion | | deletion | | | 0.9999545926710 |
| xxxx-T-024 | 43.415 | 13 bp | 22.074 | 9 bp | 21.891 | 9 bp | 0 | wild | square: |
| | | deletion | | deletion | | deletion | | type | 0.9194355944018 |
| xxxx-T-025 | 39.266 | 16 bp | 29.938 | 6 bp | 8.264 | 6 bp | 0 | wild | square: |
| | | deletion | | deletion | | deletion | | type | 0.8941856619070 |
| xxxx-T-026 | 46.506 | 22 bp | 36.554 | 7 bp | 1.953 | 16 bp | 0.058 | wild | square: |
| | | deletion | | deletion | | deletion | | type | 0.9098764137075 |
| xxxx-T-027 | 43.953 | 13 bp | 22.223 | 9 bp | 21.08 | 9 bp | 0 | wild | square: |
| | | deletion | | deletion | | deletion | | type | 0.9091420348440 |
| xxxx-T-028 | 99.815 | wild | 0.05 | 1 bp | 0.03 | 1 bp | ### | | square: |
| | | type | | Insertion | | deletion | | | 0.9999494100923 |
| xxxx-T-029 | 44.083 | 13 bp | 22.959 | 9 bp | 21.829 | 9 bp | 0 | wild | square: |
| | | deletion | | deletion | | deletion | | type | 0.9318512296710 |
| xxxx-T-030 | 34.515 | 16 bp | 32.163 | 6 bp | 6.941 | 16 bp | 0 | wild | square: |
| | | deletion | | deletion | | deletion | | type | 0.90705931481851 |
| xxxx-T-031 | 40.07 | 13 bp | 25.796 | 9 bp | 21.788 | 9 bp | 0 | wild | square: |
| | | deletion | | deletion | | deletion | | type | 0.91859146478582 |
| xxxx-T-032 | 99.891 | wild | 0.026 | 10 bp | 0.022 | 8 bp | ### | | square: |
| | | type | | deletion | | Insertion | | | 0.99995450726958 |
| xxxx-T-033 | 34.902 | 6 bp | 20.418 | 16 bp | 18.803 | 16 bp | 0 | wild | square: |
| | | deletion | | deletion | | deletion | | type | 0.91218703343041 |
| xxxx-T-034 | 38.469 | 16 bp | 33.118 | 6 bp | 8.426 | 6 bp | 0 | wild | square: |
| | | deletion | | deletion | | deletion | | type | 0.91801267185920 |

These results further demonstrate that the methods of the present invention by transient particle bombardment and indirect callus regeneration of the cells as part of preferably immature tassel are highly effective and efficient, and the methods are able to achieve single-cell origin regeneration and homogenous genome editing without a conventional selection in maize A188.

### Example 7: Efficient Genome Editing by Transient Biolistic Transformation and Indirect Callus Regeneration with Regeneration Boosters from Immature Tassel of Maize Elites

Five advanced inbred elites, including the most important pollen donor and female inbred lines, were tested for the regeneration and genome editing using the methods in the present invention via transient particle bombardment and indirect callus regeneration of the cells as part of preferably immature tassel. To this end, the elite seedlings at V7 stage, 27-30 days after planting, were harvested for immature tassel isolation.

The immature tassels were osmotically treated in N6_OSM medium for 4 hours before the bombardment. Construct pABM-BdEF1_KWS_RBP8 contains the regeneration KWS_RBP8 expression cassette (FIG. 14). For each bombardment, the two genome editing constructs (200 ng of plasmid DNA pGEP837 and 300 ng of plasmid DNA pGEP842) were co-coated with the two regeneration boost constructs (100 ng each of pABM-BdEF1_ZmPLT5 and pABM-BdEF1_KWS_RBP8) onto 100 μg of 0.6 μm gold particles using calcium-spermidine method. The four co-coated constructs were co-delivered into the maize elite immature tassel by the particle bombardment at 1, 100 psi rupture pressure, 4 bombardment shots per sample plate. For more information about the bombardment Example 2. After 20 hours of post-bombardment osmotic treatment on the N6_OSM plate, the bombarded immature tassels were cut into a segment of 3-6 mm in length, with a sharp blade, and place onto a callus induction medium IMCIM2 petro dish plate (25×100 mm). Seal the plate with surgical tape and culture at 27° C., dark, for 3 weeks. For the following steps in the indirect callus regeneration procedure, see Example 6.

After one week development in the Rooting medium in phytotray, a 5-10 mm leaf tip from each of the leaves of the regenerated plantlets were collected for DNA extraction. Genome editing SDN-1 in the regenerated T₀ plants were screened using TaqMan Digital Droplet PCR.

The results of the regeneration rates and the genome editing SDN-1 efficiencies from the tested elites were presented in Table 8. Compared to the A188, all the elites were significantly less regenerative, however all the elites tested were indeed regenerative in using the methods of in the present invention. These results indicate the possibility of genotype-independent regeneration and genome editing using by using the methods of in the present invention: that, most importantly, the methods via transient particle bombardment and indirect callus regeneration are able to achieve single-cell origin regeneration and homogenous genome editing without a conventional selection (e.g. using an antibiotic or herbicide resistant gene) directly in maize elites.

TABLE 8
SDN-1 efficiency in the regenerated T0 plantlets from a 29-day-old immature
tassel of the most important maize elites by the indirect callus regeneration
with boosters ZmPLT5 and KWS_RBP8.

| | | No. | | No. | | | | |
|---|---|---|---|---|---|---|---|---|
| | No. | Regenerated | % regen. | Mono- | No. Bi- | Total | SDN-1% | SDN-1% |
| Elite ID | tassels | (regen.) | per tassel | SDN-1 | SDN-1 | SDN-1 | per regen. | per tassel |
| FPA19-71805 | 12 | 14 | 1.2/tassel | 3 | 0 | 3 | 21.4% | 25.0% |
| FUC18-62591 | 6 | 18 | 3.0/tassel | 2 | 3 | 5 | 27.8% | 83.3% |
| WS5-33063 | 12 | 26 | 2.2/tassel | 0 | 2 | 2 | 7.7% | 16.7% |
| PJ0-73631 | 10 | 63 | 6.3/tassel | 6 | 13 | 19 | 30.2% | 190% |
| MMS18-01495 | 7 | 7 | 1.0/tassel | 0 | 4 | 4 | 57.1% | 57.1% |

### Example 8: Efficient Genome Editing by Transient Biolistic Transformation and Indirect Callus Regeneration with Regeneration Booster RBP8 from Immature Tassel of Maize Hybrids

The F1 hybrids from the reciprocal crosses between A188 and elite 4V-40171 were tested with the methods in the present invention by transient particle bombardment and indirect callus regeneration of the cells as part of preferably immature tassel. The F1 seedlings at V7 stage, 28-29 days after planting, were harvested for immature tassel isolation.

The immature tassels were osmotically treated in N6_OSM medium for 4 hours before the bombardment. Genome editing construct pGEP1067 harbors the sgRNA m7GEP22 expression cassette, which target to the maize endogenous gene HMG13 (FIG. 15). For each bombardment, the two genome editing constructs (200 ng of plasmid DNA pGEP837 and 300 ng of plasmid DNA pGEP1067) were co-coated with 100 ng of the regeneration boost construct pABM-BdEF1_KWS_RBP8 onto 100 μg of 0.6 μm gold particles using calcium-spermidine method. For more information about the bombardment, see Example 2 and Example 7. After 20 hours of post-bombardment osmotic treatment on the N6_OSM plate, the bombarded immature tassels were subjected to the indirect callus regeneration as described in Example 6 and Example 7.

After one week development in the Rooting medium in phytotray, a 5-10 mm leaf tip from each of the leaves of the regenerated plantlets were collected for DNA extraction. Genome editing SDN-1 in the regenerated T₀ plants were screened using TaqMan Digital Droplet PCR, and further confirmed by using Sanger sequencing with the genotype-specific primer (e.g, specifically amplifying the A188- or the elite-specific allele) to detect the SDN-1 in genotype-specific allele.

The regeneration rates and genome editing SDN-1 efficiencies from the hybrids are shown in Table 9.

TABLE 9
Genome editing SDN-1 at target pGEP22 (see FIG. 14) from the regenerated T0 plantlets
from a 29-day-old immature tassels of maize F1 hybrids by the indirect callus regeneration
with boosters KWS_RBP8.

| | | No. | | No. | | | | |
|---|---|---|---|---|---|---|---|---|
| | No. | Regen | | Mono- | No. Bi- | Total | SDN-1% | SDN-1% |
| Genotype | tassels | plantlets | % regen. | SDN-1 | SDN-1 | SDN-1 | per regen | per tassel |
| A188 × elite | 8 | 170 | 21.3/tassel | 8 | 14 | 22 | 12.9% | 2.75/tassel |
| elite × A188 | 4 | 41 | 10.3/tassel | 3 | 3 | 6 | 14.6% | 1.5/tassel |

Compared to the regeneration rates from A188 and the elite, the hybrids showed a regeneration capability in between, namely less regenerative than the A188, but more regenerative than the elite. Interestingly the immature tassels from the F1 seedlings derived from the cross with A188 as the female (A188×4V-40171) were significant more regenerative than those from the cross with the elite as the female. These results suggest maternal effect on plant regeneration.

Sanger sequencing with the genotype-specific primer provides an effective means to distinguish the allelic-specific SDN-1 events. The results shown in Table 10 imply that genome editing may be unbiased regarding allelic preference at the target site, and likely be allele genotype independent. The plant regeneration may be the bottleneck for plant genome modification in recalcitrant elites (Table 10).

TABLE 10
Single-cell origin SDN-1 events in the regenerated T0 plantlets from immature tassels of
maize F1 hybrids by the indirect callus regeneration with boosters KWS_RBP8 identified
via ddPCR and Sanger sequencing analyses of SDN-1.

| Regenerated | | SDN-1 via Sanger | | SDN-1 via Sanger | | SDN-1 % |
|---|---|---|---|---|---|---|
| Events | Genotype | Sequencing A188 | | Sequencing 5F | | via ddPCR |
| xxx010-T-253 | F1, elite × A188 | 3 bp | Deletion | WT | | 49.5 |
| xxx010-T-296 | F1, elite × A188 | 8 bp | deletion | 3 bp | Deletion | 99.9 |
| xxx015-T-051 | F1, A188 × elite | WT | | 9 bp | Deletion | 49.6 |
| xxx015-T-053 | F1, A188 × elite | 9 bp | Deletion | WT | | 50.9 |
| xxx015-T-067 | F1, A188 × elite | WT | | 7 bp | deletion | 48.7 |
| xxx015-T-081 | F1, A188 × elite | 7 bp | deletion | WT | | 46.2 |
| xxx016-T-037 | F1, elite × A188 | 5 bp | deletion | WT | | 33.8 |
| xxx016-T-051 | F1, elite × A188 | 7 bp | deletion | 6 bp | deletion | 93 |
| xxx016-T-081 | F1, elite × A188 | 10 bp | deletion | 6 bp | deletion | 100 |
| xxx012-T-152 | F1, elite × A188 | 8 bp | insertion | 18 bp | deletion | 99.9 |
| xxx012-T-158 | F1, elite × A188 | 8 bp | deletion | 10 bp | deletion | 100 |
| xxx012-T-171 | F1, elite × A188 | 8 bp | deletion | 3 bp | deletion | 100 |
| xxx012-T-189 | F1, elite × A188 | 8 bp | deletion | 10 bp | deletion | 100 |

The molecular analysis using TaqMan Digital Droplet PCR and Sanger sequencing with the genotype-specific allelic primer provided solid evidences in support of that the methods in the present invention are able to achieve single-cell origin regeneration and homogenous genome editing without a conventional selection in maize.

### Example 9: Stable Transformation Via Particle Bombardment and Indirect Regeneration of Immature Tassels from Maize Elites and Hybrid with a Regeneration Booster

The construct pGEP1054 harbors florescence tdTomato gene expression cassette (FIG. 16) was used for monitoring the transformation without a conventional selection. The seedlings at V7 stage, 28-30 days after planting, were harvested for immature tassel isolation. For the information on the immature tassel preparation, again see Example 1.

The immature tassels were osmotically treated in N6_OSM medium for 4 hours before the bombardment. For each bombardment, 200 ng of plasmid DNA pGEP1054 and 100 ng of plasmid DNA pABM-BdEF1_KWS_RBP8 were co-coated onto 100 μg of 0.6 μm gold particles using calcium-spermidine method. For more information about the bombardment, cf. Example 2, Example 6, and Example 7.

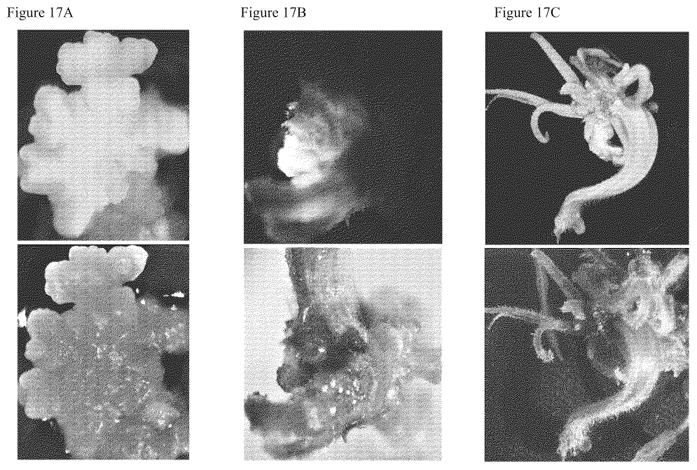

After 20 hours of post-bombardment osmotic treatment on the N6_OSM plate, the bombarded immature tassels were subjected to the callus induction at 27° C., dark, for 3 weeks (cf. Example 6 and Example 7). After 3 weeks of callus induction, the induced calluses were examined under a florescence microscope for tDTomato florescent signals. The tDTomato florescent calluses indicate foreign DNA integration and stable transformation of the tDTomato gene. The numbers of calluses showing tDTomato florescent signal were recorded and the results are summarized in Table 11. Some representative images showing stable transformation of the fluorescent report gene tDTomato in the regenerated structures are shown in FIG. 17.

These results demonstrate the feasibility of genotype-independent stable transformation via particle bombardment and regeneration of the cells as part of preferably immature tassels without a conventional selection.

TABLE 11
Stable transformation via particle bombardment and
indirect regeneration of immature tassels
from maize elites and hybrid with a regeneration.

| | No. | No. Stable | Stable tDT |
|---|---|---|---|
| Genotype | tassels | tDT events | events/per tassel |
| PJ0-73631 | 5 | 9 | 1.8/tassel |
| WS5-33063 | 8 | 7 | 0.875/tassel |
| MMS18-01495 | 5 | 4 | 0.8/tassel |
| F1 of 4V-40171 × A188 | 12 | 9 | 0.75/tassel |

### Example 10: Biolistic Transformation of Immature Inflorescences from Wheat (Triticum aestivum L.) Cultivar Taifun

### Wheat Plant Cultivation

Wheat (Triticum aestivum L.) cultivar Taifun were grown in a growth chamber. Two wheat Taifun seeds are planted into a deep inserts plug pot (placed into an 18-count holding tray without holes). After germination only one seedling per pot is kept.

The soil used was Berger 35% Bark. The seeds were germinated and grew in a growth chamber at constant 20° to 21° C., with light intensity of 400-600 μmol m² s⁻¹ and 14 hours day length from September to April, and 16 hours day length from May to August. The humidity was 40%-60%. The wheat plants were fertilized three times a week with Jack's 15-16-17 peat lite at an E.C, of 1.0+the E.C, of the water. The plants were checked twice a day for watering needs and were watered from top as needed.

### Wheat Immature Inflorescence (Spike) Isolation

The developmental stages of wheat immature inflorescences were determined using a Zeiss stereo microscope. When the first node of stem was visible the inflorescences of a wheat plant were defined to be in the DR (double ridge/spikelet meristem stage) stage. About 1 week later, the second node is usually emerging, floret meristem development begins, and the plant is in then in the FM (floret meristem) stage. After 5-7 more days, the third stem internode begins to elongate, and anther primordia become visible, and the plant is then in AM (anther primordium) stage and is ready to enter the booting stage.

Wheat immature inflorescences at the development stages of late double ridge (DR) to late anther meristem (AM) are used for the methods in the present invention. At these stages the wheat shoots are elongated with 1-3 visible nodes.

The wheat immature inflorescence isolation comprises the steps of:
1. Harvesting the wheat shoots at the right development stages from the base of the stalks (near to soil) and remove all the leaf blades;
2. (Optional) Rinsing the stem segments with tap water, and dry with paper towel;
3. Surface spraying the shoots with 70% ethanol, and manually removing the first leaf sheath from stalk in a laminar hood;
4. Repeating step 3, and carefully removing each leaf sheaths from stalk, one by one from the bottom to top, until the flag leaf sheath;
5. Trimming the stalk segments and spraying with 70% ethanol (the last ethanol spray);
6. Transferring the segments onto a clear petri dish in the hood;
7. Under a dissection microscope: carefully removing the flag leaf sheath, and all of immature bracts, for providing an immature inflorescence/spike is ready for transformation.

### Microparticle Bombardment

Freshly isolated wheat immature spikes were osmotically treated in N6OSM medium for 2-4 hours, as also detailed in Example 2. Three plasmids were co-bombarded, which were: construct GEMT121 (SEQ ID NO: 50) that contains the expression cassettes of the fluorescent report gene tDTomato and CRISPR nuclease LbCpf1 (FIG. 18), GEMT099 (SEQ ID NO: 51) that harbors the expression cassette of CRISPR sgRNA crGEP289 targeting to wheat CPL3 (C-terminal domain phosphatase-like 3) gene (FIG. 19), and construct pABM-BdEF1_KWS_RBP8 (SEQ ID NO: 42) that encloses boost gene KWS_RBP8 expression cassette (FIG. 14). Specifically, 200 ng of plasmid DNA GEMT121, 300 ng of plasmid DNA GEMT099, and 100 ng of pABM-BdEF1_KWS_RBP8, were co-coated onto 100 μg of 0.6 μm gold particles using calcium-spermidine method, and the three constructs were co-delivered into the cells of wheat immature inflorescence meristem by particle bombardment at 900 psi rupture pressure. Two-row spikes of wheat Taifun immature inflorescence were arranged vertically with one-row side inserted onto the osmotic N6OSM medium. Totally six bombardment shots per sample plate were conducted (3 shots per each row-side of the spikes). After bombardment, wheat Taifun immature spikes were kept on the osmotic N60SM plate flatly for another 16-20 hours, and then the bombarded spikes were cut into 2-5 mm segments and transferred onto an indirect callus regeneration medium, e.g. N6_5Ag for callus induction at 27° C., dark, for 3 weeks. For details, see Example 6, Example 7, and Example 9.

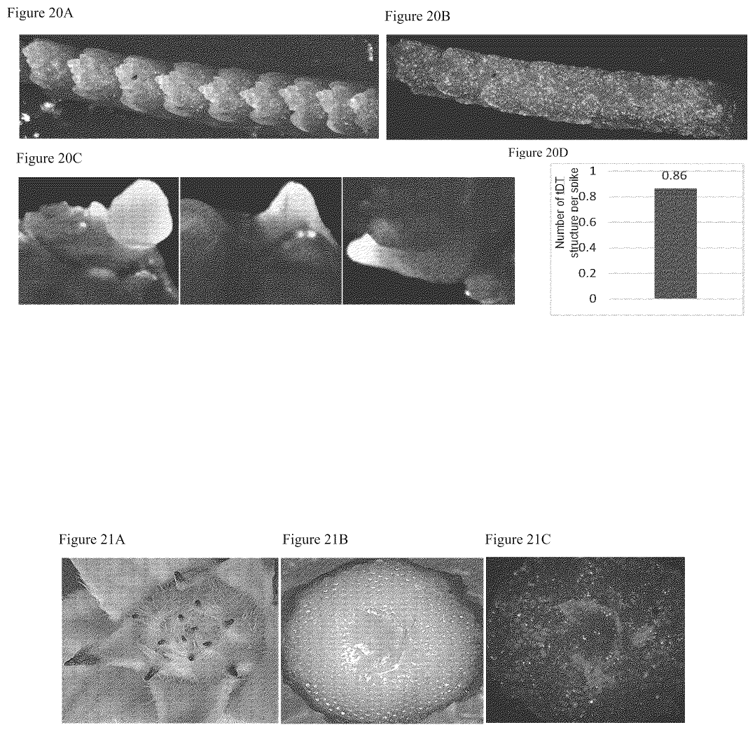

Biolistic transformation efficiency was monitored by observing the fluorescence tDTomato expression under a microscope 16-20 hours after bombardment. Efficient transformation of the tDTomato in the cells from wheat immature spike was demonstrated, as shown in FIG. 20B.

tDTomato florescent signals in the bombarded immature spike were monitored under a florescence microscope along the callus induction process. Strong and constant tDTomato florescent signals from the growing tips of the bombarded spikelet appeared 3 days after bombardment, indicating stable transformation of the tDTomato gene. The representative results are shown FIG. 20C. The numbers of tDTomato florescent growing structure were presented in FIG. 20D.

These results demonstrate the feasibility of the present methods for rapid and efficient genome modification in wheat.

### Example 11: Efficient Genome Editing by Transient Biolistic Transformation and Indirect Callus Regeneration from Wheat Immature Spike

After 16-20 hours of post-bombardment osmotic treatment on the N6_OSM plate, the bombarded wheat immature tassels were subjected to the indirect callus regeneration as detailed in Example 6 and Example 7.

After one week of development in the Rooting medium in phytotray, a 5-10 mm leaf tip from each of the leaves of the regenerated plantlets was collected for DNA extraction. Genome editing SDN-1 in the regenerated T₀ wheat plants were screened using TaqMan Digital Droplet PCR, and further confirmed by Sanger sequencing.

### Example 12: Biolistic Transformation of Immature Inflorescences from Sunflower (Helianthus annuus) Cultivar Velvet Queen

### Sunflower Plant Cultivation:

Sunflower (Helianthus annuus) cultivar velvet Queen were grown in a growth house. Two sunflower velvet Queen seeds were planted into a deep inserts plug pot (placed into an 18-count holding tray without holes). After germination only one seedling per pot was kept.

The soil used was MetroMix360/Turface 3:1 blend. The seeds were germinated and grown in a growth chamber at 25° C., for day and 22° C., for night, light intensity of 400-600 μmol m² s⁻¹ and 14 hours day length, 50% humidity. Sunflower plants are fertilized at every watering using Jack's 15-5-15 Ca—Mg diluted to 150 ppm nitrogen. Plants were watered as needed.

### Sunflower Development Stages:

1. Vegetative Emergence (VE): Seedling has emerged and the first leaf beyond the cotyledons is less than 4 cm long.
2. Vegetative stages (V): These are determined by counting the number of true leaves at least 4 cm in length beginning as V-1, V-2, V-3, V-4, etc.
3. Reproductive stage 1 (R1): The terminal bud forms a miniature floral head rather than a cluster of leaves. When viewed from directly above the immature bracts form a many-pointed star-like appearance.
4. Reproductive stage 2 (R2): The immature bud elongates 0.5 to 2.0 cm above the nearest leaf attached to the stem.
5. Reproductive stage 3 (R3): The immature bud elongates more than 2.0 cm above the nearest leaf.
6. Reproductive stage 4 (R4): The inflorescence begins to open. When viewed from directly above immature ray flowers are visible.

### Sunflower Immature Inflorescence (Head) Isolation:

Sunflower immature inflorescence head from the plants at R1 stages (FIG. 21A) were used. It most likely takes around 30-45 days to reach these stages for different genotypes after seed planting when cultured at the growth conditions described above. Sunflower immature inflorescence head isolation usually comprises the steps of
1. Harvesting the inflorescence head from sunflower plants at R1 stage by cutting the stem above the nearest leaf attached to;
2. (Optional) Rinsing the stalk segments with tap water, and dry with paper towel;
3. Surface spraying the stalk segments with 70% ethanol, and manually removing the leaves;
4. Trimming the stalk heads, carefully removing all remaining young leaves and spraying them with 70% ethanol
5. Transferring the heads onto a clear petri dish in the laminar hood;
6. Under a dissection microscope, carefully removing all bracts to expose the immature inflorescence head, so that the immature tassels are ready for transformation (FIG. 21B).

### For Bombardment Method and Fluorescence Observation and Imaging Please See Example 2 and Example 10.

### Example 13: Media

The following media were used for the above Examples. As it is known to the skilled person, variations to the media composition can be made depending on the target cells or tissues to be treated and depending on selection criteria. A variety of suitable media is available in the art for a given plant, cell, tissue, or organ to be treated and/or cultivated.

IM_OS: MS salt; LS vitamins; 1× FeEDTA: 100 mg/L casein: 0.5 mg/L kinetin: 30 g/L sucrose, 36.4 g/L of Mannitol, 36.4 g/L of sorbitol: 7 g/L of Gelzan: pH: 5.8.

N6OSM: N6 salts and vitamin, 100 mg/L of Caseine, 0.7 g/L of L-proline, 0.2 M Mannitol (36.4 g/L), 0.2 M sorbitol (36.4 g/L), 20 g/L sucrose, 15 g/L of Bacto-agar, pH 5.8.

IMSMK5: 1× MS salt, 1× KM vitamins, 1× FeEDTA, 1.25 mg/L CuSO4.5H2O, 1.0 g/L of KNO3, 2.0 mg/L Dicamba, 3.0 mg/L BAP, 0.5 mg/L Kinetin, 0.5 g/L of MES, 3% sucrose, 3 g/L Gelzan, pH: 5.8.

IMCIM2: MS salt, LS vitamins, 1.0 g/L of Proline, 5 mg/L Dicamba, 1.0 mg/L 2,4 D, 0.2 mg/L of BAP, 0.5 mg/L kinetin, 1.0 g/L of KNO3, 2.0 mg/L of AgNO3, 3% sucrose, 3 g/L gelrite, pH: 5.8.

N6_5Ag: N6 salt and vitamin, 1.0 mg/L of 2, 4-D, 100 mg/L of Caseine, 2.9 g/L of L-proline, 20 g/L sucrose, 5 g/L of glucose, 5 mg/L of AgNO3, 8 g/L of Bacto-agar, pH 5.8.

Shooting medium: 1× MS salt, 1× LS vitamins, 1× FeEDTA, 2.5 mg/L CuSO4.5H2O, 100 mg/L Myo Inosit, 5 mg/L Zeatin, 0.5 g/L of MES, 20 g/L of sucrose, 3 g/L Gelzan, pH: 5.8.

Rooting medium: 1× MS salts, LS vitamins, 1× FeEDTA, 0.5 mg/L MES, 0.5 mg/L IBA, 1.25 mg/L of CuSO4, 20 g/L sucrose, 3 g/L Gelzan.

### Example 14: Efficient Plant Regeneration from the Cross-Section Discs of Immature Tassel Center Spike in Corn A188

Tassel inflorescence consists of a symmetrical, many-rowed central axis (center spike) and several asymmetrical, two-ranked branches (branch tassels) (FIG. 3A). Compared to tassel branches, the center spike is relatively large and a major part of tassel inflorescence. However, it is also relatively challenging to obtain even distribution of gold particles in biolistic bombardment of the tassel center spike due to its cylinder shape. To better serve as an explant for biolistic transformation, a tassel center spike can be further cross-sectioned into thin discs for plant transformation and regeneration to increase utilization efficiency.

It is particularly important for some maize elites that the initiation and development of axillary branches are significantly behind that of the center spike, so that the immature tassels therefore consist almost solely of center spike when harvested. The use of cross-section discs of immature center spike is an efficient solution for such maize genotypes.

For information about immature tassel preparation, see examples 1A and 1B. After isolation, under aseptic condition, central tassels are laid onto Whitman filter paper saturated with 1× N6 buffer in a petri dish, quickly cross-sectioned into thin discs about 0.5 mm in depth with a sharp razorblade and transferred onto an osmotic medium plate (N6OSM) immediately for 4 hours of pre-bombardment osmotic treatment.

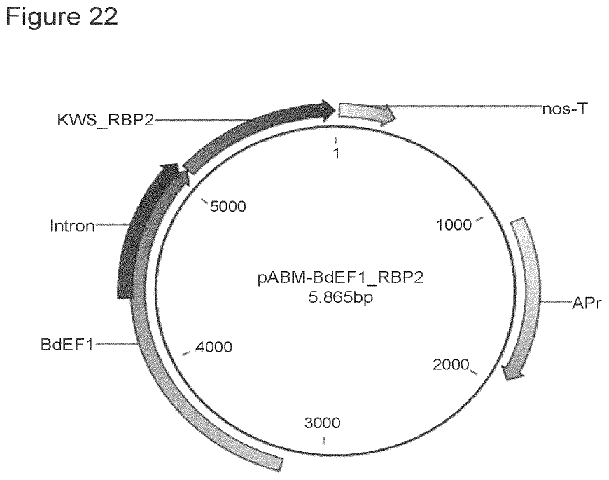

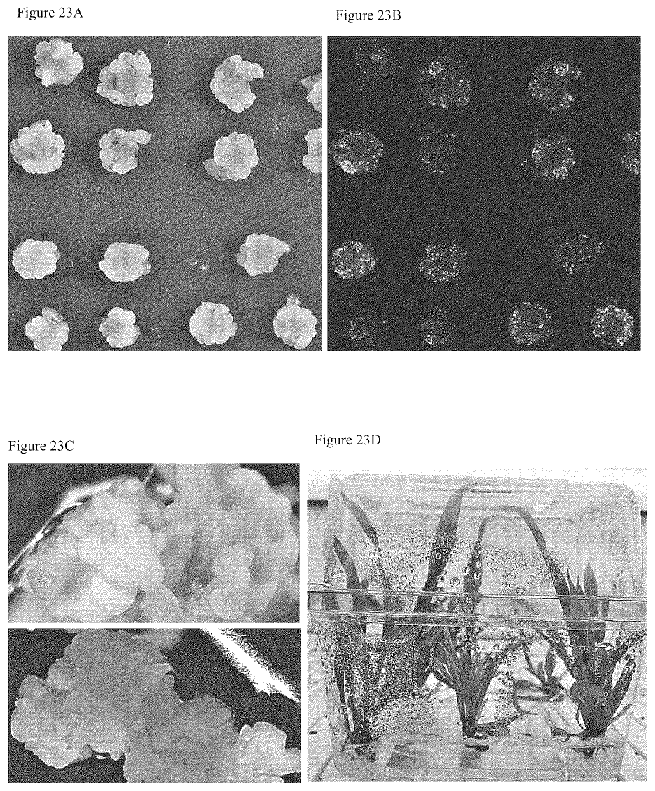

For information about biolistic bombardment and indirect callus regeneration, see examples 2 and 6, respectively. Specifically, 100 ng of plasmid pGEP1054 (containing the fluorescence report gene tDTomato expression cassette, FIGS. 16) and 100 ng of pABM-BdEF1_KWS_RBP2 harboring the regeneration boost gene KWS_RBP2 expression cassette (FIG. 22) were co-coated onto 100 μg of 0.6 μm gold particles using the calcium-spermidine method. For more information about the bombardment, see example 2. After 18 hours of post-bombardment osmotic treatment on the N6OSM plate, the bombarded immature tassels were subjected to indirect callus regeneration as described in example 6 and 7.

FIG. 23 shows a representative experiment, where two central tassels were cross-sectioned into ˜0.5 mm discs (FIG. 23A), and from which 86 plants were regenerated. It's worth to note that the tDTomato fluorescence signals are mostly derived from the outer ring of discs that is collocated with the spikelet pair meristems (SPM) ring (FIG. 23 B). This result suggests that meristematic cells are suitable for biolistic bombardment.

### Example 15: Efficient Multiplex Genome Editing Via Co-Bombardment and Indirect Callus Regeneration A188 Immature Tassel

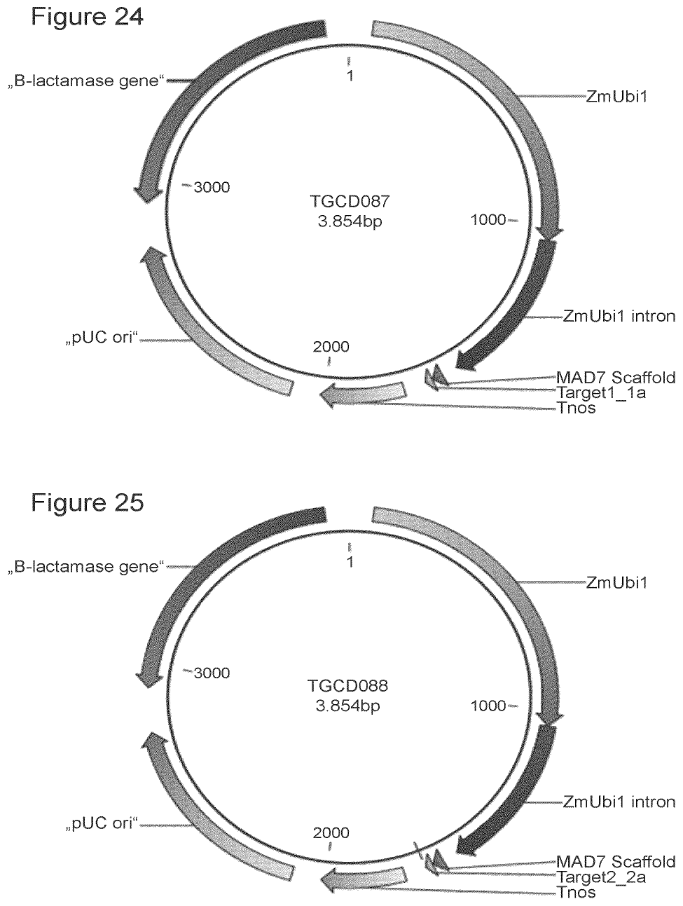

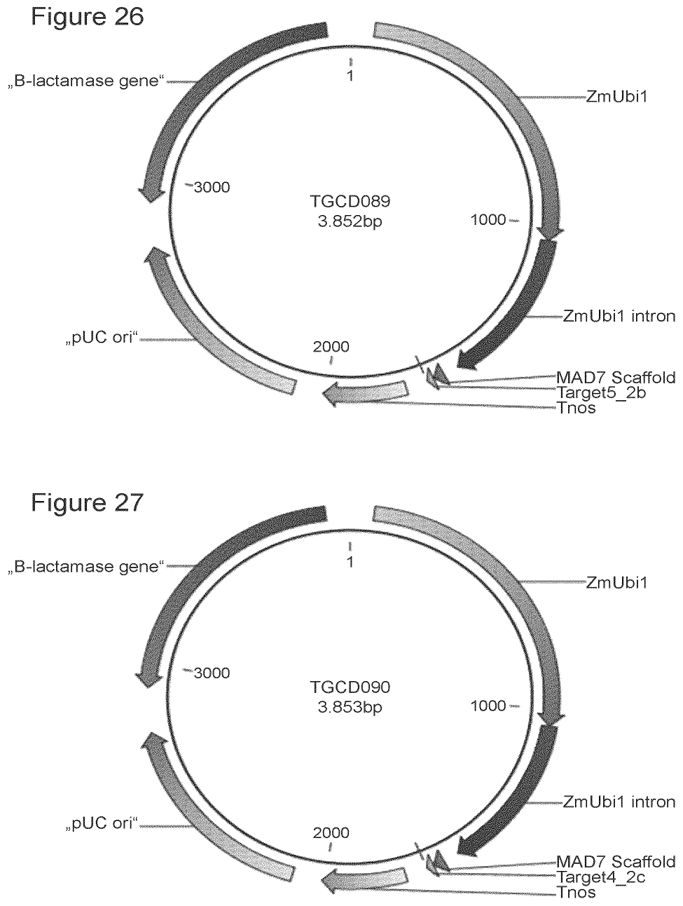

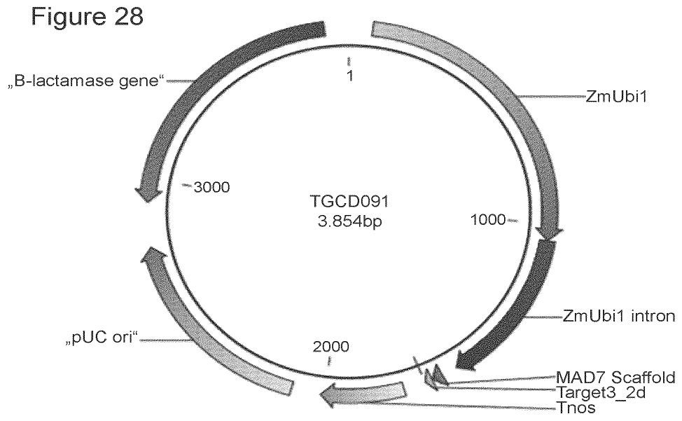

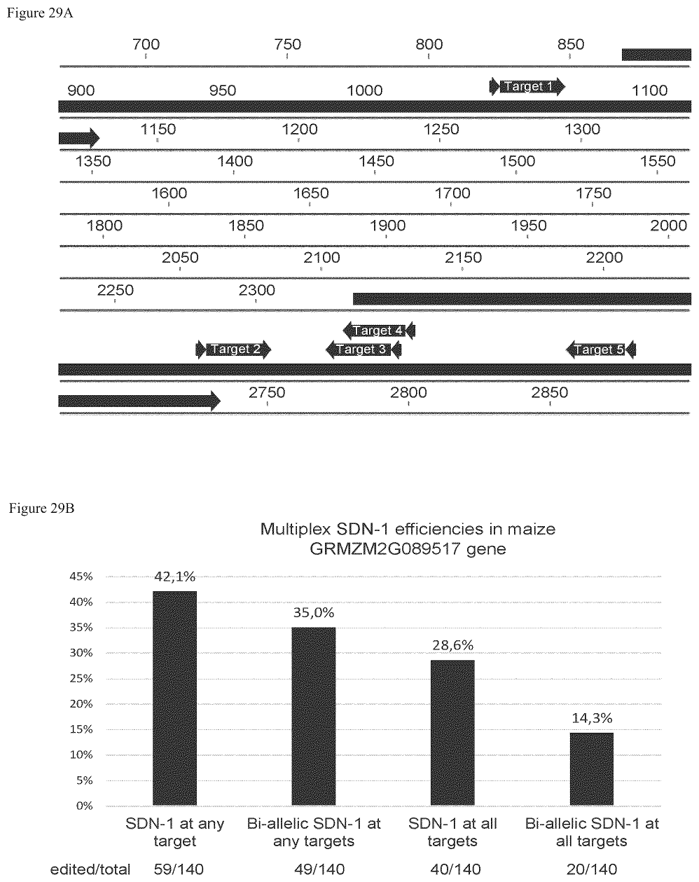

For information about immature tassel preparation and particle bombardment, see examples 1 and 2. Specifically, co-bombardment consists of 7 plasmids as follows:
100 ng of genome editing construct pGEP1054 harboring CRISPR nuclease MAD7 nuclease expression cassette (FIG. 16)
150 ng each of five guide RNA constructs, which code five CRISPR guide RNA expression cassettes, and target to five locations in the maize target gene annotated as UV-B-insensitive 4-like gene (FIG. 29A).
   TGCD087 (FIG. 24), coding the Target 1 guide RNA
   TGCD088 (FIG. 25), coding the Target 2 guide RNA
   TGCD089 (FIG. 26), coding the Target 5 guide RNA
   TGCD090 (FIG. 27) coding the Target 4 guide RNA
   TGCG091 (FIG. 28), coding the Target 3 guide RNA
100 ng of pABM-BdEF1_KWS_RBP2 harboring the regeneration boost gene KWS_RBP2 expression cassette.

For each co-bombardment, seven of the above-mentioned plasmids (pGEP1054, TGCG087 to TGCD091, and pABM-BdEF1_KWS_RBP2) were co-coated onto 100 μg of 0.6 μm gold particles using the calcium-spermidine method. For more information about the bombardment, see example 2. After 18 hours of post-bombardment osmotic treatment on the N6OSM plate, bombarded immature tassels were subjected to indirect callus regeneration as described in example 6 and 7. 140 TO plants were regenerated. After one week of development in the rooting medium in phytotray, a 5-10 mm leaf tip from each of the regenerated plant leaves were collected for DNA extraction. Genome editing SDN-1 in the regenerated TO plants were screened by Sanger sequencing and sequencing trace decomposition analysis. Multiplex genome editing SDN-1 efficiency in the maize target gene from the TO regenerated A188 plants are summarized in FIG. 29 B. 14.3% of the TO plants have a bi-allelic SDN-1 modification at all the five target sequences. These results further demonstrate that the methods of the present invention by transient biolistic co-bombardment and indirect callus regeneration of cells as part of preferably immature tassel is highly effective and efficient, and the methods are able to achieve highly efficient genome editing in multiple locations simultaneously.

1-20. (canceled)

21. An isolated regeneration booster polypeptide (RBP) encoded by an isolated nucleic acid sequence, wherein the nucleic acid sequence comprises a sequence selected from any one of SEQ ID NOs: 1 to 8, or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to the respective sequence of SEQ ID NOs: 1 to 8 with the proviso that the sequence encodes a regeneration booster with the same function as the respective reference sequence, wherein the polypeptide comprises a sequence selected from any one of SEQ ID NOs: 12 to 19, or a sequence having at least 90%, 91%, 92%, 93%, 94%, 95%, 96%, 97%, 98%, 99% identity to the respective sequence of SEQ ID NOs: 12 to 19 with the proviso that the sequence has regeneration booster function as the respective reference sequence.

22. An expression cassette or an expression construct comprising a sequence encoding a regeneration booster polypeptide according to claim 21.

23-24. (canceled)
